(19) United States
(12) Patent Application Publication
(10) Pub. No.: US 2022/0204994 A1
(43) Pub. Date: Jun. 30, 2022

(54) METHODS OF PREPARING POPULATIONS OF GENETICALLY-MODIFIED IMMUNE CELLS

(71) Applicant: Precision BioSciences, Inc., Durham, NC (US)

(72) Inventors: Tyler Goodwin, Durham, NC (US); Aaron Martin, Carrboro, NC (US); Melissa Samo, Raleigh, NC (US)

(73) Assignee: Precision BioSciences, Inc., Durham, NC (US)

(21) Appl. No.: 17/601,415

(22) PCT Filed: Apr. 3, 2020
(86) PCT No.: PCT/US2020/026551

§ 371 Date: Oct. 4, 2021

Related U.S. Application Data
(60) Provisional application No. 62/987,805, filed on Mar. 10, 2020.
(60) Provisional application No. 62/950,683, filed on Dec. 19, 2019.
(60) Provisional application No. 62/908,905, filed on Oct. 1, 2019.
(60) Provisional application No. 62/829,945, filed on Apr. 5, 2019.

(51) Int. Cl. C12N 15/88; C12N 5/0783; C12N 15/62

(52) U.S. Cl. CPC C12N 15/88; C12N 5/0636; C12N 15/625; C12N 2501/515; C12N 2501/2302; C12N 2510/00; C12N 2500/36

(57) ABSTRACT

The present disclosure provides methods for preparing a population of genetically-modified immune cells. The methods include contacting a population of immune cells with lipid nanoparticles in the presence of an apolipoprotein. The lipid nanoparticles include mRNA encoding an engineered nuclease having specificity for a recognition sequence in the genome of the immune cells. The mRNA is delivered into the immune cells and the engineered nuclease is expressed, generating a cleavage site at the recognition sequence. Further provided are populations of genetic ally-modified immune cells produced according to the disclosed methods, pharmaceutical compositions containing such cells, and methods of treating diseases with the genetically-modified immune cells.

### FIELD OF THE INVENTION

The present invention generally relates to the field of oncology, cancer immunotherapy, molecular biology, nanotechnology, and recombinant nucleic acid technology. In particular, the invention relates to a simplified method for introducing mRNA encoding an engineered nuclease into immune cells, such as T cells or natural killer (NK) cells.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 2, 2020, is named P109070036WO00-SEQ-EPG, and is 6 kilobytes in size.

### BACKGROUND OF THE INVENTION

Genetic modification of human T cells is being leveraged for a number of therapeutic approaches, including the development of T cells expressing chimeric antigen receptors (CARs) or exogenous T cell receptors (TCRs). T cells expressing chimeric antigen receptors (CAR T cells) induce tumor immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Typically, a coding sequence for a CAR or TCR is introduced into the cell by a viral vector. In some cases, the coding sequence is randomly integrated into the genome of the cell using a lentiviral vector. Insertion of the CAR or TCR coding sequence can be accompanied by the use of an engineered nuclease to knock out certain genes of interest. For example, to produce a CAR T cell useful for allogeneic administration, an engineered nuclease can be used to knock out expression of an endogenous TCR (e.g., an alpha/beta TCR). CAR T cells expressing an endogenous T cell receptor may recognize major and minor histocompatibility antigens following administration to an allogeneic patient, which can lead to the development of graft-versus-host-disease (GVHD).

In other cases, the coding sequence is specifically inserted in a target gene. Generally, the process of targeted insertion is made possible by the use of an engineered nuclease which generates a double-stranded cleavage site in the genome at the target gene. The CAR or TCR coding sequence is then inserted at the cleavage site by homologous recombination, resulting in expression of the transgene while disrupting expression of the protein encoded by the target gene.

Engineered nucleases are usually introduced into T cells using mRNA. However, it is well established that primary T cells are notoriously difficult to transfect with nucleic acids. In order to introduce mRNA encoding a nuclease, T cells generally undergo a process of electroporation. This method exposes T cells to a number of electrical and mechanical stresses that impact cell viability, number, and proliferation in the aftermath of the process.

Furthermore, when a template comprising a CAR or TCR coding sequence is also introduced, this is often done by contacting the T cells with an adeno-associated virus (AAV) comprising the template. Methods that include both the introduction of a nucleic acid encoding a nuclease, and the introduction of a CAR or TCR coding sequence, often require a number of centrifugation, buffer change, and vessel transfer steps that further impact recovery and performance of the cell population.

Accordingly, there remains a need in the art for additional methods of transfection that allow for simplified introduction of mRNA into primary immune cells without producing the negative effects associated with current methods.

### SUMMARY OF THE INVENTION

The present invention provides a simplified method for introducing mRNA encoding an engineered nuclease into immune cells, such as T cells or natural killer (NK) cells. The method can be used alone for the purpose of knocking out a gene of interest in immune cells, such as genes encoding components of a TCR. Alternatively, the method can be used in concert with the introduction of a template nucleic acid encoding a protein (e.g., a CAR or exogenous TCR) that is inserted at the nuclease cleavage site by homology-directed repair, thus disrupting expression of a polypeptide encoded by the target gene while allowing for expression of the exogenous protein.

Generally, the methods of the invention comprise the use of lipid nanoparticles (LNPs) comprising mRNA encoding an engineered nuclease. LNPs particularly useful for in the present methods comprise a cationic lipid selected from DLin-DMA, DLin-MC3-DMA, DLin-KC2-DMA, DODMA, SS-OP, and derivatives thereof. Herein is disclosed that contacting immune cells (e.g., T cells) with such LNPs in the presence of an apolipoprotein (e.g., within a composition comprising the immune cells and the LNPs) allows for efficient uptake and expression of the mRNA, subsequent gene editing and disruption, and/or targeted insertion of a donor template encoding a protein of interest (e.g., a CAR or exogenous TCR) at the nuclease cleavage site. Surprisingly, according to the present disclosure, it has been found that the inclusion of an apolipoprotein, such as ApoE, dramatically improved the efficiency of this process and the resulting genetically-modified immune cells exhibited improved properties.

Accordingly, in one aspect, the invention provides a method for preparing genetically-modified immune cells by contacting immune cells with lipid nanoparticles in the presence of an apolipoprotein. For example, the immune cells and the lipid nanoparticles can be contacted within a composition comprising the apolipoprotein. The lipid nanoparticles comprise a cationic lipid selected from the group consisting of DLin-DMA, DLin-MC3-DMA, DLin-KC2-DMA, DODMA, SS-OP, and derivatives thereof. Further, the lipid nanoparticles comprise mRNA encoding an engineered nuclease having specificity for a recognition sequence in the genome of the immune cells. According to the method, the mRNA is delivered into the immune cells and the engineered nuclease is expressed, wherein the nuclease generates a cleavage site at the recognition sequence.

In some embodiments of the method, the immune cells are contacted with the lipid nanoparticles in a serum-free culture condition.

In some embodiments of the method, the immune cells are contacted with the lipid nanoparticles in a culture condition comprising serum at a concentration (vol/vol) of less than about 0.31%, less than about 0.625%, less than about 1.25%, less than about 2.5%, less than about 5%, or less than about 10%. In some embodiments of the method, the immune cells are contacted with the lipid nanoparticles in a culture condition comprising serum at a concentration (vol/vol) of from about 0% to about 0.31%, from about 0% to about 0.625%, from about 0% to about 1.25%, from about 0% to about 2.5%, from about 0% to about 5%, or from about 0% to about 10%.

In some embodiments, the method is performed in vitro.

In some embodiments of the method, the immune cells are human immune cells.

In some embodiments of the method, the immune cells are T cells, or cells derived therefrom, natural killer (NK) cells, or cells derived therefrom, or B cells, or cells derived therefrom.

In some embodiments of the method, the apolipoprotein is present (e.g., in a composition comprising the immune cells and the lipid nanoparticles) at a concentration between 0.01 μg/mL to 10 μg/mL (e.g., μg per mL of culture medium). In particular embodiments of the method, the apolipoprotein is present at a concentration of about 1 μg/mL (e.g., μg per mL of culture medium).

In some embodiments of the method, the apolipoprotein is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)) protein. In particular embodiments of the method the apolipoprotein is ApoE. In some embodiments, ApoE is ApoE2, ApoE3, or ApoE4. In certain embodiments, ApoE is ApoE2. In particular embodiments, ApoE is ApoE3. In other embodiments, ApoE is ApoE4.

In some embodiments of the method, the lipid nanoparticles do not comprise an immune cell targeting molecule.

In some embodiments of the method, the recognition sequence is in a target gene, and expression of a polypeptide encoded by the target gene is disrupted by non-homologous end joining at the cleavage site.

In certain embodiments of the method, the target gene is selected from the group consisting of a TCR alpha gene, a TCR alpha constant region gene, a TCR beta gene, a TCR beta constant region gene, a beta-2 microglobulin gene, a CD52 gene, a CS1 (i.e., SLAMF7 or CD319) gene, a Cbl proto-oncogene B (CBL-B) gene, a CD52 gene, a CD7 gene, a programmed cell death-1 (PD-1) gene, a lymphocyte-activation 3 (LAG-3) gene, a transforming growth factor beta receptor II (TGFBRII) gene, a T-cell immunoglobulin and mucin-domain containing-3 (TIM-3) gene, a T cell immunoreceptor with Ig and ITIM domains (TIGIT) gene, a CD70 gene, a glucocorticoid receptor gene, a Tet methylcytosine dioxygenase 2 (TET2) gene, a general control nonderepressible 2 (GCN2) gene, a deoxycytidine kinase (DCK) gene, a cytotoxic T-lymphocyte associated protein 4 (CTLA-4) gene, or a C-C motif chemokine receptor 5 (CCR5) gene. In particular embodiments of the method, the target gene is a TCR alpha gene. In particular embodiments of the method, the target gene is a TCR alpha constant region gene. In some such embodiments, the genetically-modified immune cells do not have detectable cell-surface expression of an endogenous TCR (e.g., an alpha/beta TCR).

In some embodiments, the method produces a population of genetically-modified immune cells wherein between about 5% and about 70% of the genetically-modified immune cells in the population do not have detectable cell-surface expression of an endogenous TCR (e.g., an alpha/beta TCR). In some embodiments, the method produces a population of genetically-modified immune cells wherein between about 5% and about 70% of the genetically-modified immune cells in the population comprise an inactivated TCR alpha gene.

In some embodiments of the method, the genetically-modified immune cells express a chimeric antigen receptor (CAR) or exogenous TCR.

In some embodiments of the method, the immune cells are contacted with: (a) a first population of lipid nanoparticles comprising mRNA encoding a first engineered nuclease having specificity for a first recognition sequence; and (b) a second population of lipid nanoparticles comprising mRNA encoding a second engineered nuclease having specificity for a second recognition sequence; wherein the first engineered nuclease and the second engineered nuclease are expressed in the immune cells, and wherein the first engineered nuclease generates a first cleavage site in the first recognition sequence and the second engineered nuclease generates a second cleavage site in the second recognition sequence. In some such embodiments, the first recognition sequence and the second recognition sequence are in the same target gene, and expression of a polypeptide encoded by the target gene is disrupted by non-homologous end joining at the first cleavage site and the second cleavage site. In other such embodiments, the first recognition sequence and the second recognition sequence are in different target genes, wherein expression of polypeptides encoded by the different target genes is disrupted by non-homologous end joining at the first cleavage site and the second cleavage site. In some such embodiments, the different target genes are a human TCR alpha constant region gene and a human beta-2 microglobulin gene, wherein the genetically-modified immune cells do not have detectable cell-surface expression of an endogenous TCR (e.g., an alpha/beta TCR) or beta-2 microglobulin.

In some embodiments, the method further comprises introducing into the immune cells a template nucleic acid comprising an exogenous polynucleotide, wherein the exogenous polynucleotide is inserted into the genome of the immune cells at the cleavage site.

In some such embodiments of the method, the recognition sequence is in a target gene, and insertion of the exogenous polynucleotide disrupts expression of a polypeptide encoded by the target gene.

In some such embodiments of the method, the target gene is selected from the group consisting of a TCR alpha gene, a TCR alpha constant region gene, a TCR beta gene, a TCR beta constant region gene, a beta-2 microglobulin gene, a CD52 gene, a CS1 (i.e., SLAMF7 or CD319) gene, a Cbl proto-oncogene B (CBL-B) gene, a CD52 gene, a CD7 gene, a programmed cell death-1 (PD-1) gene, a lymphocyte-activation 3 (LAG-3) gene, a transforming growth factor beta receptor II (TGFBRII) gene, a T-cell immunoglobulin and mucin-domain containing-3 (TIM-3) gene, a T cell immunoreceptor with Ig and ITIM domains (TIGIT) gene, a CD70 gene, a glucocorticoid receptor gene, a Tet methylcytosine dioxygenase 2 (TET2) gene, a general control nonderepressible 2 (GCN2) gene, a deoxycytidine kinase (DCK) gene, a cytotoxic T-lymphocyte associated protein 4 (CTLA-4) gene, or a C-C motif chemokine receptor 5 (CCR5) gene. In certain embodiments of the method, the target gene is a TCR alpha gene. In certain embodiments of the method, the target gene is a TCR alpha constant region gene. In certain embodiments of the method, the target gene is a TCR alpha constant region gene, and the genetically-modified immune cells do not have detectable cell-surface expression of an endogenous TCR (e.g., an alpha/beta TCR).

In some such embodiments of the method, the exogenous polynucleotide encodes a polypeptide of interest. In certain embodiments of the method, the exogenous polynucleotide encodes a CAR or an exogenous TCR.

In some such embodiments of the method, the template nucleic acid is introduced into the immune cells using a recombinant DNA construct. In certain embodiments of the method, the recombinant DNA construct is encapsulated in a lipid nanoparticle.

In some such embodiments of the method, the template nucleic acid is introduced into the immune cells using a recombinant virus. In certain embodiments of the method, the recombinant virus is recombinant adenovirus, a recombinant lentivirus, a recombinant retrovirus, or a recombinant adeno-associated virus (AAV). In particular embodiments, the recombinant virus is a recombinant AAV.

In some such embodiments of the method, the template nucleic acid is introduced into the immune cells within 48 hours after the immune cells are contacted with the lipid nanoparticles. In certain embodiments of the method, the template nucleic acid is introduced into the immune cells between 0-24 hours or between 24-48 hours after the immune cells are contacted with the lipid nanoparticles. In some embodiments, the template nucleic acid is introduced into said immune cells within 12 hours prior to when said immune cells are contacted with said lipid nanoparticles.

In some such embodiments of the method, the immune cells are not transferred to a new vessel between the step of contacting with the lipid nanoparticles and the step of introducing the template nucleic acid. In certain embodiments of the method, the immune cells are not centrifuged between the step of contacting with the lipid nanoparticles and the step of introducing the template nucleic acid.

In some such embodiments of the method, the genetically-modified immune cells are genetically-modified T cells, or cells derived therefrom, expressing a chimeric antigen receptor or exogenous TCR. In certain embodiments of the method, the genetically-modified T cells do not have detectable cell-surface expression of an endogenous TCR (e.g., an alpha/beta TCR).

In some embodiments, the method produces a population of genetically-modified T cells having a CD4+ T cell to CD8+ T cell ratio of between about 0.8 and about 1.6 when cultured for one to two weeks after the step of contacting the immune cells with the lipid nanoparticles.

In some embodiments, the method produces a population of genetically-modified T cells wherein between about 65% and about 84% of CD4+ T cells in the population exhibit a central memory phenotype when cultured for one to two weeks after the step of contacting the immune cells with the lipid nanoparticles.

In some embodiments, the method produces a population of genetically-modified T cells wherein about 3% to about 10% of CD4+ T cells in the population exhibit an effector phenotype when cultured for one to two weeks after the step of contacting the immune cells with the lipid nanoparticles.

In some embodiments of the method, the molar concentration of the cationic lipid is from about 20% to about 80%, from about 30% to about 70%, from about 40% to about 60%, from about 45% to about 55%, or about 50% of the total lipid molar concentration, wherein the total lipid molar concentration is the sum of the cationic lipid and other lipid component molar concentrations. In certain embodiments of the method, the molar concentration of the cationic lipid is about 40%, about 50%, or about 60% of the total lipid molar concentration.

In some embodiments of the method, the lipid nanoparticles comprise a molar ratio of cationic lipid to mRNA of from about 1 to about 20, from about 2 to about 16, from about 4 to about 12, from about 6 to about 10, or about 8. In certain embodiments of the method, the lipid nanoparticles comprise a molar ratio of cationic lipid to mRNA of about 8.

In some embodiments of the method, the lipid nanoparticles comprise: (a) one or more non-cationic lipids; and (b) a lipid conjugate.

In some embodiments of the method, the molar concentration of the non-cationic lipids is from about 20% to about 80%, from about 30% to about 70%, from about 40% to about 70%, from about 40% to about 60%, from about 46% to about 50%, or about 48.5% of the total lipid molar concentration. In certain embodiments of the method, the molar concentration of the non-cationic lipids is about 40%, about 48.5%, about 50%, or about 60% of the total lipid molar concentration.

In some embodiments of the method, the non-cationic lipids comprise a phospholipid, wherein the molar concentration of the phospholipid is from about 0% to about 30%, from about 2.5% to about 25%, from about 5% to about 20%, from about 5% to about 15%, from about 7.5% to about 12.5%, or about 10% of the total lipid molar concentration. In certain embodiments of the method, the molar concentration of the phospholipid is about 10% or about 20% of the total lipid molar concentration.

In certain embodiments of the method, the phospholipid is DSPC.

In some embodiments of the method, the non-cationic lipids comprise a steroid, wherein the molar concentration of the steroid is from about 20% to about 60%, from about 25% to about 55%, from about 30% to about 50%, from about 35% to about 40%, or about 38.5% of the total lipid molar concentration. In certain embodiments of the method, the molar concentration of the steroid is about 30%, about 38.5%, or about 50% of the total lipid molar concentration.

In particular embodiments of the method, the steroid is cholesterol.

In some embodiments of the method, the molar concentration of the lipid conjugate is from about 0.01% to about 10%, from about 0.2% to about 8%, from about 0.5% to about 5%, from about 0.1% to about 1.5%, from about 1% to about 2%, or about 1.5% of the total lipid molar concentration. In certain embodiments of the method, the molar concentration of the lipid conjugate is about 1.5% of the total lipid molar concentration.

In certain embodiments of the method, the lipid conjugate is a pegylated lipid. In particular embodiments of the method, the lipid conjugate is a DMG-PEG. In certain embodiments of the method, the lipid conjugate is DMG-PEG2000 or DMG-PEG5000.

In some embodiments of the method, the lipid nanoparticles have a size from about 50 nm to about 300 nm, or from about 60 nm to about 120 nm. In some embodiments of the method, the polydispersity index of the lipid nanoparticles is less than about 0.3, or less than about 0.2. In some embodiments of the method, the zeta potential of the lipid nanoparticles is from about −40 mV to about 40 mV, or from about −10 mV to about 10 mV.

In some embodiments of the method, a molar ratio of the cationic lipid to the phospholipid is from about 1:1 to about 20:1, about 6:1 to about 20:1, about 10:1 to about 20:1, about 16:1 to about 20:1, or about 2:1 to about 7:1. In some of these embodiments a molar ratio of the cationic lipid to the phospholipid is from about 2:1 to about 7:1. In some of these embodiments, a molar ratio of the cationic lipid to the phospholipid is about 2:1, about 4:1, about 5:1, or about 6:1.

In some embodiments of the method, a molar ratio of the cationic lipid to the steroid is from about 0.25:1 to about 5:1, about 0.5:1 to about 5:1, about 0.75:1 to about 5:1, about 2:1 to about 5:1, or about 0.8:1 to about 2:1. In some of these embodiments, a molar ratio of the cationic lipid to the steroid is from about 0.8:1 to about 2:1. In some of these embodiments, a molar ratio of the cationic lipid to the steroid is about 0.8:1, about 1.3:1, about 1:1, or about 2:1.

In some embodiments of the method, a molar ratio of the cationic lipid to the lipid conjugate is from about 10:1 to about 1000:1, about 25:1 to about 1000:1, about 75:1 to about 1000:1, about 400:1 to about 1000:1, about 550:1 to about 1000:1, about 20:1 to about 600:1, or about 25:1 to about 400:1. In some of these embodiments, a molar ratio of the cationic lipid to the lipid conjugate is from about 25:1 to about 400:1. In some of these embodiments, a molar ratio of the cationic lipid to the lipid conjugate is about 25:1, about 33:1, about 60:1, or about 400:1.

In some embodiments of the method, a molar ratio of the steroid to the lipid conjugate is from about 25:1 to about 750:1, about 50:1 to about 750:1, about 100:1 to about 750:1, about 150:1 to about 750:1, about 200:1 to about 750:1, about 250:1 to about 750:1, about 300:1 to about 750:1, about 350:1 to about 750:1, about 400:1 to about 750:1, about 450:1 to about 750:1, about 500:1 to about 750:1, about 10:1 to about 500:1, or about 25:1 to about 500:1. In some of these embodiments, a molar ratio of the steroid to the lipid conjugate is from about 25:1 to about 500:1. In some of these embodiments, a molar ratio of the steroid to the lipid conjugate is from about 25:1, about 30:1, or about 500:1.

In some embodiments of the method, a molar ratio of the phospholipid to the lipid conjugate is from about 1:1 to about 300:1, about 50:1 to about 300:1, about 100:1 to about 300:1, about 125:1 to about 300:1, about 150:1 to about 300:1, about 175:1 to about 300:1, about 200:1 to about 300:1, about 225:1 to about 300:1, about 250:1 to about 300:1, about 275:1 to about 300:1, about 3:1 to about 200:1, or about 5:1 to about 100:1. In some of these embodiments, a molar ratio of the phospholipid to the lipid conjugate is from about 5:1 to about 100:1. In some embodiments, a molar ratio of the phospholipid to the lipid conjugate is about 6:1, about 10:1, about 13:1 or about 100:1.

In some embodiments of the method, the lipid nanoparticles comprise: (a) the cationic lipid at a molar concentration of about 30% to about 60% of the total lipid molar concentration; (b) a steroid at a molar concentration of about 20% to about 60% of the total lipid molar concentration; (c) a phospholipid at a molar concentration of about 5% to about 20% of the total lipid molar concentration; and (d) a lipid conjugate at a molar concentration of about 0.10% to about 1.5% of the total lipid molar concentration.

In some embodiments of the method, the lipid nanoparticles comprise: (a) the cationic lipid at a molar concentration of about 40% the total lipid molar concentration; (b) a steroid at a molar concentration of about 38.5% of the total lipid molar concentration; (c) a phospholipid at a molar concentration of about 20% of the total lipid molar concentration; and (d) a lipid conjugate at a molar concentration about 1.5% of the total lipid molar concentration.

In some embodiments of the method, the lipid nanoparticles comprise: (a) the cationic lipid at a molar concentration of about 50% of the total lipid molar concentration; (b) a steroid at a molar concentration of about 38.5% of the total lipid molar concentration; (c) a phospholipid at a molar concentration about 10% of the total lipid molar concentration; and (d) a lipid conjugate at a molar concentration of about 1.5% of the total lipid molar concentration.

In some embodiments of the method, the lipid nanoparticles comprise: (a) the cationic lipid at a molar concentration of about 60% of the total lipid molar concentration; (b) a steroid at a molar concentration of about 29% of the total lipid molar concentration; (c) a phospholipid at a molar concentration of about 10% of the total lipid molar concentration; and (d) a lipid conjugate at a molar concentration of about 1% of the total lipid molar concentration.

In some embodiments of the method, the lipid nanoparticles comprise: (a) a cationic lipid at a molar concentration of about 40% of the total lipid molar concentration; (b) a steroid at a molar concentration of about 48.5% of the total lipid molar concentration; (c) a phospholipid at a molar concentration of about 10% of the total lipid molar concentration; and (d) a lipid conjugate at a molar concentration of about 1.5% of the total lipid molar concentration.

In some embodiments of the method, the lipid nanoparticles comprise: (a) the cationic lipid at a molar concentration of about 40% of the total lipid molar concentration; (b) a steroid at a molar concentration of about 49.9% of the total lipid molar concentration; (c) a phospholipid at a molar concentration of about 10% of the total lipid molar concentration; and (d) a lipid conjugate at a molar concentration of about 0.10% of the total lipid molar concentration.

In some embodiments of the method, the cationic lipid is DLin-DMA, or derivatives thereof. In some embodiments of the method, the cationic lipid is DLin-MC3-DMA, or derivatives thereof. In some embodiments of the method, the cationic lipid is DLin-KC2-DMA, or derivatives thereof. In some embodiments of the method, the cationic lipid is DODMA, or derivatives thereof. In some embodiments of the method, the cationic lipid is SS-OP, or derivatives thereof.

In some embodiments of the method, the cationic lipid is DLin-MC3-DMA, the steroid is cholesterol, the phospholipid is DSPC, and the lipid conjugate is PEG 5000.

In some embodiments of the method, the cationic lipid is DLin-MC3-DMA, the steroid is cholesterol, the phospholipid is DSPC, and the lipid conjugate is PEG 2000.

In some embodiments of the method, the cationic lipid is DLin-MC3-DMA, the steroid is cholesterol, the phospholipid is DOPC, and the lipid conjugate is PEG 2000.

In some embodiments of the method, the cationic lipid is DLin-KC2-DMA, the steroid is cholesterol, the phospholipid is DSPC, and the lipid conjugate is PEG 5000.

In some embodiments of the method, the cationic lipid is DLin-KC2-DMA, the steroid is cholesterol, the phospholipid is DSPC, and the lipid conjugate is PEG 2000.

In some embodiments of the method, the cationic lipid is DLin-KC2-DMA, the steroid is cholesterol, the phospholipid is DOPC, and the lipid conjugate is PEG 2000.

In some embodiments of the method, the cationic lipid is DLin-DMA, the steroid is cholesterol, the phospholipid is DSPC, and the lipid conjugate is PEG 5000.

In some embodiments of the method, the cationic lipid is DLin-DMA, the steroid is cholesterol, the phospholipid is DSPC, and the lipid conjugate is PEG 2000.

In some embodiments of the method, the cationic lipid is DLin-DMA, the steroid is cholesterol, the phospholipid is DOPC, and the lipid conjugate is PEG 2000.

In some embodiments of the method, the cationic lipid is SS-OP, the steroid is cholesterol, the phospholipid is DSPC, and the lipid conjugate is PEG 5000.

In some embodiments of the method, the cationic lipid is SS-OP, the steroid is cholesterol, the phospholipid is DSPC, and the lipid conjugate is PEG 2000.

In some embodiments of the method, the cationic lipid is SS-OP, the steroid is cholesterol, the phospholipid is DOPC, and the lipid conjugate is PEG 2000.

In some embodiments of the method, the cationic lipid is DODMA, the steroid is cholesterol, the phospholipid is DSPC, and the lipid conjugate is PEG 5000.

In some embodiments of the method, the cationic lipid is DODMA, the steroid is cholesterol, the phospholipid is DSPC, and the lipid conjugate is PEG 2000.

In some embodiments of the method, the cationic lipid is DODMA, the steroid is cholesterol, the phospholipid is DOPC, and the lipid conjugate is PEG 2000.

In some embodiments of the method, the lipid nanoparticles comprise DLin-MC3-DMA, DSPC, cholesterol, and DMG-PEG2000 at a molar ratio of about 50:10:38.5:1.5 or about 40:10:48.5:1.50. In some embodiments of the method, the lipid nanoparticles comprise DLin-MC3-DMA, DSPC, cholesterol, and DMG-PEG2000 at a molar ratio of about 50:10:38.5:1.5. In some embodiments of the method, the lipid nanoparticles comprise DLin-MC3-DMA, DSPC, cholesterol, and DMG-PEG2000 at a molar ratio of about 40:10:48.5:1.50.

In some embodiments of the method, the lipid nanoparticles comprise DLin-MC3-DMA, DSPC, cholesterol, and DMG-PEG5000 at a molar ratio of about 40:10:49.90:0.10.

In some embodiments of the method, the lipid nanoparticles comprise DLin-MC3-DMA, DOPC, cholesterol, and DMG-PEG2000 at a molar ratio of about 40:20:38.5:1.5 or about 60:10:29:1. In some embodiments of the method, the lipid nanoparticles comprise DLin-MC3-DMA, DOPC, cholesterol, and DMG-PEG2000 at a molar ratio of about 40:20:38.5:1.5. In some embodiments of the method, the lipid nanoparticles comprise DLin-MC3-DMA, DOPC, cholesterol, and DMG-PEG2000 at a molar ratio of about 60:10:29:1.

In some embodiments of the method, the lipid nanoparticles comprise DODMA, DSPC, cholesterol, and DMG-PEG2000 at a molar ratio of about 50:10:38.5:1.5.

In some embodiments of the method, the lipid nanoparticles can be any one of the compositions according to Table 1.

In some embodiments of the method, the engineered nuclease is an engineered meganuclease, a zinc finger nuclease, a TALEN, a compact TALEN, a CRISPR system nuclease, or a megaTAL. In certain embodiments of the method, the engineered nuclease is an engineered meganuclease.

In some embodiments of the method, the lipid nanoparticles do not comprise a T cell targeting molecule.

In some embodiments of the method, the mRNA comprises a 5′ cap. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof.

In some embodiments of the method, the mRNA comprises at least one nucleoside modification. In certain embodiments of the method, the nucleoside modification is selected from the group consisting of a modification from uridine to pseudouridine and uridine to N1-methyl pseudouridine. In particular embodiments of the method the nucleoside modification is from uridine to pseudouridine.

In some embodiments of the method, the mRNA does not comprise a nucleoside substitution.

In another aspect, the invention provides a population of genetically-modified immune cells prepared according to any of the methods described herein.

In another aspect, the invention provides a population of genetically-modified immune cells that are electroporation naïve, wherein the genetically-modified immune cells comprise a target gene modified by an engineered nuclease to disrupt expression of an endogenous polypeptide encoded by the target gene.

In some embodiments, the genetically-modified immune cells are genetically-modified T cells, genetically-modified NK cells, or genetically-modified B cells. In certain embodiments, the genetically-modified immune cells are genetically-modified human T cells.

In some embodiments, the genetically-modified immune cells further comprise a nucleic acid sequence encoding a CAR or an exogenous TCR, wherein the CAR or exogenous TCR is expressed by the genetically-modified immune cell.

In another aspect, the invention provides a population of immune cells, wherein between about 5% and about 80%, between about 10% and about 80%, between about 20% and about 80%, between about 30% and about 80%, between about 40% and about 80%, between about 50% and about 80%, between about 55% and about 80%, between about 60% and about 80%, between about 65% and about 80%, between about 70% and about 80%, or between about 75% and about 80% of the immune cells in the population are genetically-modified immune cells prepared by the methods described herein, wherein the genetically-modified immune cells comprise a disrupted TCR alpha gene or a disrupted TCR alpha constant region gene.

In certain embodiments of these populations, the genetically-modified immune cells are genetically-modified T cells, genetically-modified NK cells, or genetically-modified B cells. In particular embodiments of these populations, the genetically-modified immune cells are genetically-modified human T cells.

In another aspect, the invention provides a population of immune cells, wherein between about 5% and about 65%, between about 10% and about 65%, between about 20% and about 65%, between about 30% and about 65%, between about 40% and about 65%, between about 45% and about 65%, between about 50% and about 65%, between about 55% and about 65%, or between about 60% and about 65% of the immune cells in the population are genetically-modified immune cells prepared by the methods described herein, wherein the genetically-modified immune cells comprise a disrupted TCR alpha gene or a disrupted TCR alpha constant region gene and express a chimeric antigen receptor or an exogenous TCR.

In certain embodiments of these populations, the genetically-modified immune cells are genetically-modified T cells, genetically-modified NK cells, or genetically-modified B cells. In particular embodiments of these populations, the genetically-modified immune cells are genetically-modified human T cells.

In another aspect, the invention provides a pharmaceutical composition comprising a pharmaceutically-acceptable carrier and a population of genetically-modified immune cells described herein. In another aspect, the invention provides a pharmaceutical composition comprising a pharmaceutically-acceptable carrier and a population of immune cells described herein that comprises genetically-modified immune cells described herein.

In another aspect, the invention provides a method of treating a disease in a subject in need thereof, wherein the method comprises administering to the subject a therapeutically-effective amount of the population of genetically-modified immune cells described herein, or an effective amount of the population of immune cells described herein that comprises genetically-modified immune cells described herein. In certain embodiments, the method comprises administering to the subject a pharmaceutical composition described herein.

In some embodiments of the method, the method is an immunotherapy for the treatment of a cancer in a subject in need thereof, wherein the genetically-modified immune cells are genetically-modified human T cells, or cells derived therefrom, or genetically-modified NK cells, or cells derived therefrom, and wherein the genetically-modified immune cells express a CAR or an exogenous TCR, and wherein the genetically-modified immune cells do not have detectable cell-surface expression of an endogenous TCR (e.g., an alpha/beta TCR).

In some embodiments of the method, the cancer is selected from the group consisting of a cancer of carcinoma, lymphoma, sarcoma, blastomas, and leukemia. In certain embodiments of the method, the cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma. In particular embodiments of the method, the cancer of B-cell origin is selected from the group consisting of B-lineage acute lymphoblastic leukemia, B-cell chronic lymphocytic leukemia, B-cell non-Hodgkin's lymphoma, and multiple myeloma.

In another aspect, the invention provides genetically-modified immune cells, or populations thereof, described herein for use as a medicament. The invention further provides the use of genetically-modified immune cells or populations thereof described herein in the manufacture of a medicament for treating a disease in a subject in need thereof. In one such aspect, the medicament is useful for cancer immunotherapy in subjects in need thereof.

In another aspect, the invention provides a lipid nanoparticle composition comprising: (a) a cationic lipid at a molar concentration of about 40%, about 50%, or about 60% of the total lipid molar concentration, wherein the cationic lipid is selected from the group consisting of DLin-MC3-DMA, DLin-KC2-DMA, DODMA, SS-OP, and derivatives thereof; (b) a steroid at a molar concentration of about 29%, about 38.5%, about 48.5%, or about 49.9% of the total lipid molar concentration; (c) a phospholipid at a molar concentration of about 10% or about 20% of the total lipid molar concentration; and (d) a lipid conjugate at a molar concentration of about 0.10% or about 1.5% of the total lipid molar concentration.

In some embodiments of the composition, the lipid nanoparticles comprise: (a) the cationic lipid at a molar concentration of about 40% of the total lipid molar concentration; (b) the steroid at a molar concentration of about 38.5% of the total lipid molar concentration; (c) the phospholipid at a molar concentration of about 20% of the total lipid molar concentration; and (d) the lipid conjugate at a molar concentration of about 1.5% of the total lipid molar concentration.

In some embodiments of the composition, the lipid nanoparticles comprise: (a) the cationic lipid at a molar concentration of about 50% of the total lipid molar concentration; (b) the steroid at a molar concentration of about 38.5% of the total lipid molar concentration; (c) the phospholipid at a molar concentration of about 10% of the total lipid molar concentration; and (d) the lipid conjugate at a molar concentration of about 1.5% of the total lipid molar concentration.

In some embodiments of the composition, the lipid nanoparticles comprise: (a) the cationic lipid at a molar concentration of about 60% of the total lipid molar concentration; (b) the steroid at a molar concentration of about 29% of the total lipid molar concentration; (c) the phospholipid at a molar concentration of about 10% of the total lipid molar concentration; and (d) the lipid conjugate at a molar concentration of about 1% of the total lipid molar concentration.

In some embodiments of the composition, the lipid nanoparticles comprise: (a) the cationic lipid at a molar concentration of about 40% of the total lipid molar concentration; (b) the steroid at a molar concentration of about 48.5% of the total lipid molar concentration; (c) the phospholipid at a molar concentration of about 10% of the total lipid molar concentration; and (d) the lipid conjugate at a molar concentration of about 1.5% of the total lipid molar concentration.

In some embodiments of the composition, the lipid nanoparticles comprise: (a) the cationic lipid at a molar concentration of about 40% of the total lipid molar concentration; (b) the steroid at a molar concentration of about 49.9% of the total lipid molar concentration; (c) the phospholipid at a molar concentration of about 10% of the total lipid molar concentration; and (d) the lipid conjugate at a molar concentration of about 0.10% of the total lipid molar concentration.

In some embodiments of the composition, a molar ratio of the cationic lipid to the steroid is about 0.8:1, about 1.3:1, about 1:1, or about 2:1. In some of these embodiments, a molar ratio of the cationic lipid to the phospholipid is from about 2:1, about 4:1, about 5:1, or about 6:1. In some of these embodiments, a molar ratio of the cationic lipid to the lipid conjugate is about 25:1, about 33:1, about 60:1, or about 400:1. In some of these embodiments, a molar ratio of the steroid to the lipid conjugate is from about 25:1, about 30:1, or about 500:1. In some of these embodiments, a molar ratio of the phospholipid to the lipid conjugate is about 6:1, about 10:1, about 13:1 or about 100:1.

In some embodiments of the composition, the cationic lipid is DLin-MC3-DMA, the steroid is cholesterol, the phospholipid is DSPC, and the lipid conjugate is PEG 5000.

In some embodiments of the composition, the cationic lipid is DLin-MC3-DMA, the steroid is cholesterol, the phospholipid is DSPC, and the lipid conjugate is PEG 2000.

In some embodiments of the composition, the cationic lipid is DLin-MC3-DMA, the steroid is cholesterol, the phospholipid is DOPC, and the lipid conjugate is PEG 2000.

In some embodiments of the composition, the lipid nanoparticles comprise DLin-MC3-DMA, DSPC, cholesterol, and DMG-PEG2000 at a molar ratio of about 50:10:38.5:1.5 or about 40:10:48.5:1.50.

In some embodiments of the composition, the lipid nanoparticles comprise DLin-MC3-DMA, DSPC, cholesterol, and DMG-PEG5000 at a molar ratio of about 40:10:49.90:0.10.

In some embodiments of the composition, the lipid nanoparticles comprise DLin-MC3-DMA, DOPC, cholesterol, and DMG-PEG2000 at a molar ratio of about 40:20:38.5:1.5 or about 60:10:29:1.

In some embodiments of the composition, the lipid nanoparticles comprise DODMA, DSPC, cholesterol, and DMG-PEG2000 at a molar ratio of about 50:10:38.5:1.5.

In some embodiments of the composition, the lipid nanoparticles can be any one of the compositions according to Table 1.

In some embodiments of the composition, the lipid nanoparticles further comprise an mRNA encoding an engineered nuclease having specificity for a recognition sequence in the genome of an immune cell.

In some embodiments of the composition, the mRNA comprises a 5′ cap. In some embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. In some embodiments, the mRNA comprises at least one nucleoside modification. In some embodiments, the nucleoside modification is selected from the group consisting of a modification from uridine to pseudouridine and uridine to N1-methyl pseudouridine. In some embodiments, the nucleoside modification is from uridine to pseudouridine.

In some embodiments of the composition, the mRNA does not comprise a nucleoside substitution.

In some embodiments of the composition, the lipid nanoparticles have a size from about 50 nm to about 300 nm or from about 60 nm to about 120 nm.

In some embodiments of the composition, the polydispersity index of the lipid nanoparticles is less than about 0.3 or less than about 0.2.

In some embodiments of the composition, the zeta potential of the lipid nanoparticles is from about −40 mV to about 40 mV or from about −10 mV to about 10 mV.

In some embodiments of the composition, the lipid nanoparticles comprise a molar ratio of cationic lipid to mRNA of from about 1 to about 20, from about 2 to about 16, from about 4 to about 12, from about 6 to about 10, or about 8. In some embodiments, the lipid nanoparticles comprise a molar ratio of cationic lipid to mRNA of about 8.

In some embodiments of the composition, the lipid nanoparticles do not comprise an immune cell targeting molecule. In certain embodiments of the composition, the lipid nanoparticles do not comprise a T cell targeting molecule.

In another aspect, the invention provides a kit for transfecting a eukaryotic cell with mRNA comprising: (a) an apolipoprotein and (b) any lipid nanoparticle composition as described herein. In some embodiments of the kit, the apolipoprotein is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)) protein. In some embodiments of the kit, the apolipoprotein is ApoE. In some embodiments, ApoE is ApoE2, ApoE3, or ApoE4. In certain embodiments, ApoE is ApoE2. In particular embodiments, ApoE is ApoE3. In other embodiments, ApoE is ApoE4. In some embodiments of the kit, the apolipoprotein and the lipid nanoparticle composition are provided together in one vial or are provided separately in two or more vials.

### BRIEF DESCRIPTION OF THE DRAWINGS

FIG. 1 illustrates total live cell counts for LNP transfected cells, with and without ApoE, as compared to electroporated cells.

FIG. 2 illustrates eGFP⁺ cell counts for LNP transfected cells, with and without ApoE, as compared to electroporated cells.

FIG. 3 illustrates % eGFP⁺ cells for LNP transfected cells, with and without ApoE, as compared to electroporated cells.

FIG. 4 illustrates the mean fluorescence intensity (MFI) for eGFP⁺ cells for LNP transfected cells, with and without ApoE, as compared to electroporated cells.

FIG. 5 illustrates eGFP⁺ cell counts for LNP transfected cells, with and without ApoE, as compared to electroporated cells after 72 hours.

FIG. 6 illustrates CD3 knockout cell counts for LNP transfected cells as compared to electroporated cells after 48 hours.

FIG. 7 illustrates eGFP⁺ cell % for invivofectamine LNP transfected cells as compared to electroporated cells. A) Mock-transfected cells. B) Cells transfected by electroporation. C) Cells transfected using invivofectamine LNP.

FIG. 8 illustrates CD3 knockout frequency for invivofectamine LNP transfected cells as compared to electroporated cells. A) Electroporated cell population selected for analysis by front and side scattering (FSC and SSC) B) CD3 knockout following electroporation. C) Invivofectamine-treated cell population selected for analysis by front and side scattering (FSC and SSC) D) CD3 knockout following transfection with invivofectamine LNP.

FIG. 9 illustrates CD3 knockout frequency for MC3 and DODMA LNP transfected cells as compared to electroporated cells at day 3. A) Mock-transfected cells. B) Cells transfected by electroporation. C) Cells transfected with a DODMA LNP. D) Cells transfected with an MC3 LNP.

FIG. 10 illustrates CD3 knockout frequency for MC3 and DODMA LNP transfected cells as compared to electroporated cells at day 7. A) Mock-transfected cells. B) Cells transfected by electroporation. C) Cells transfected with a DODMA LNP. D) Cells transfected with an MC3 LNP.

FIG. 11 illustrates CD3 knockout frequency for MC3 and DODMA LNP transfected cells as compared to electroporated cells at day 9. A) Mock-transfected cells. B) Cells transfected by electroporation. C) Cells transfected with a DODMA LNP. D) Cells transfected with an MC3 LNP.

FIG. 12 is a tabular summary of results for Example 4, illustrating CD3 knockout on day 3, day 7, and day 9 post-transfection by electroporation or MC3 LNP.

FIG. 13 illustrates cell distribution following various methods of transfection with or without apolipoprotein. A) CD4+ and CD8+ cell populations following mock-transfection. B) CD3 knockout following mock-transfection. C) CD4+ and CD8+ cell populations following transfection by electroporation. D) CD3 knockout following transfection by electroporation. E) CD4+ and CD8+ cell populations following transfection by MC3 LNP in the presence of apolipoprotein. F) CD3 knockout following transfection by MC3 LNP in the presence of apolipoprotein. G) CD4+ and CD8+ cell populations following transfection by MC3 LNP in the absence of apolipoprotein. H) CD3 knockout following transfection by MC3 LNP in the absence of apolipoprotein.

FIG. 14 illustrates the production of CD3−/CAR+ T cells following mRNA transfection with an MC3 LNP and subsequent transduction with the CAR AAV within 0-24 hours, 24-48 hours, 48-72 hours, or 72-96 hours. A) Day 3 following transfection with an MC3 LNP and no transduction with AAV. B) Day 8 following transfection with an MC3 LNP and no transduction with AAV. C) Day 10 following transfection with an MC3 LNP and no transduction with AAV. D) Day 3 following transfection with an MC3 LNP and subsequent transduction with a CAR AAV within 0-24 hours. E) Day 8 following transfection with an MC3 LNP and subsequent transduction with a CAR AAV within 0-24 hours. F) Day 10 following transfection with an MC3 LNP and subsequent transduction with a CAR AAV within 0-24 hours. G) Day 3 following transfection with an MC3 LNP and subsequent transduction with a CAR AAV within 24-48 hours. H) Day 8 following transfection with an MC3 LNP and subsequent transduction with a CAR AAV within 24-48 hours. I) Day 10 following transfection with an MC3 LNP and subsequent transduction with a CAR AAV within 24-48 hours. J) Day 3 following transfection with an MC3 LNP and subsequent transduction with a CAR AAV within 48-72 hours. K) Day 8 following transfection with an MC3 LNP and subsequent transduction with a CAR AAV within 48-72 hours. L) Day 10 following transfection with an MC3 LNP and subsequent transduction with a CAR AAV within 48-72 hours. M) Day 8 following transfection with an MC3 LNP and subsequent transduction with a CAR AAV within 72-96 hours. N) Day 10 following transfection with an MC3 LNP and subsequent transduction with a CAR AAV within 72-96 hours.

FIG. 15 is a tabular summary showing the optimization of time points for AAV addition after LNP transfection.

FIG. 16 illustrates flow cytometry analysis of CD19+ cancer cell killing 16 h post co-culturing with anti-CD19 CAR T cells, generated at various time points after LNP transfection and AAV transduction. A) Results using cells transfected with an MC3 LNP but no AAV transduction. B) Results using cells transfected with an MC3 LNP and transduced with a CAR AAV within 0-24 hours. C) Results using cells transfected with an MC3 LNP and transduced with a CAR AAV within 24-48 hours. D) Results using cells transfected with an MC3 LNP and transduced with a CAR AAV within 48-72 hours.

FIG. 17 illustrates flow cytometry analysis of CD4+ and CD8+ T cell populations at different time points after transfection and/or transduction. A) Day 4 following transfection by electroporation and no AAV transduction. B) Day 7 following transfection by electroporation and no AAV transduction. C) Day 12 following transfection by electroporation and no AAV transduction. D) Day 4 following transfection by electroporation and CAR AAV transduction. E) Day 7 following transfection by electroporation and CAR AAV transduction. F) Day 12 following transfection by electroporation and CAR AAV transduction. G) Day 4 following transfection by MC3 LNP and no AAV transduction. H) Day 7 following transfection by MC3 LNP and no AAV transduction. I) Day 12 following transfection by MC3 LNP and no AAV transduction. J) Day 4 following transfection by MC3 LNP and CAR AAV transduction. K) Day 7 following transfection by MC3 LNP and CAR AAV transduction. L) Day 12 following transfection by MC3 LNP and CAR AAV transduction.

FIG. 18 illustrates flow cytometry analysis comparing the frequency of CD3−/CAR+ cells produced following transfection of mRNA using electroporation or LNPs followed by transduction with a CAR AAV. A) Day 4 following transfection by electroporation and no AAV transduction. B) Day 7 following transfection by electroporation and no AAV transduction. C) Day 12 following transfection by electroporation and no AAV transduction. D) Day 4 following transfection by electroporation and CAR AAV transduction. E) Day 7 following transfection by electroporation and CAR AAV transduction. F) Day 12 following transfection by electroporation and CAR AAV transduction. G) Day 4 following transfection by MC3 LNP and no AAV transduction. H) Day 7 following transfection by MC3 LNP and no AAV transduction. I) Day 12 following transfection by MC3 LNP and no AAV transduction. J) Day 4 following transfection by MC3 LNP and CAR AAV transduction. K) Day 7 following transfection by MC3 LNP and CAR AAV transduction. L) Day 12 following transfection by MC3 LNP and CAR AAV transduction.

FIG. 19 illustrates flow cytometry analysis comparing T cell memory phenotype populations produced following transfection by electroporation or LNP and subsequent transduction with a CAR AAV. A) Memory phenotype of mock-transfected CD4+ cells. B) Memory phenotype of mock-transfected CD8+ cells. C) Memory phenotype of CD4+ cells following transfection by electroporation but no transduction by AAV. D) Memory phenotype of CD8+ cells following transfection by electroporation but no transduction by AAV. E) Memory phenotype of CD4+ cells following transfection by electroporation and transduction with a CAR AAV. F) Memory phenotype of CD8+ cells following transfection by electroporation and transduction with a CAR AAV. G) Memory phenotype of CD4+ cells following transfection by MC3 LNP and no transduction by AAV. H) Memory phenotype of CD8+ cells following transfection by electroporation and no transduction by AAV. I) Memory phenotype of CD4+ cells following transfection by MC3 LNP and transduction with a CAR AAV. J) Memory phenotype of CD8+ cells following transfection by electroporation and transduction with a CAR AAV.

FIG. 20 is a tabular summary of results comparing electroporation and LNP transfection.

FIG. 21 illustrates flow cytometry analysis of B2M gene knockout in T cells following transfection by electroporation, single transfection by LNP, or repeated transfection by LNP. A) B2M knockout frequency in mock-transfected cells. B) B2M knockout frequency in cells following transfection by electroporation. C) B2M knockout frequency in cells following a single transfection by an MC3 LNP. D) B2M knockout frequency in cells following transfection by an MC3 LNP on day 0 and day 3.

FIG. 22 illustrates flow cytometry analysis of a double knockout of the endogenous TCR (i.e., CD3− cells) and B2M proteins following sequential MC3 LNP transfections and transduction with a CAR AAV to generate CAR+/CD3−/B2M− T cells. A) TCR knockout following MC3 LNP transfection and CAR AAV transduction. B) B2M knockout following MC3 LNP transfection and CAR AAV transduction. C) Frequency of CAR+ T cells in the CD3− cell population (shown in A) following MC3 LNP transfection and CAR AAV transduction. D) Frequency of B2M− T cells in the CAR+/CD3− cell population (shown in C) following MC3 LNP transfection and CAR AAV transduction.

FIG. 23 provides a table summarizing formulations tested in T-cell transfection.

FIG. 24 provides a table summarizing the number, percentage, and return on investment (ROI) of CD3− cells 7 days and 10 days post transfection from the initial input of 1E5 T cells.

FIG. 25 provides a table summarizing additional formulations tested in T-cell transfection. The number, percentage, and ROI of CD3− cells 10 days post transfection is shown.

FIG. 26 provides a table summarizing the number and percent of CD3− cells and the knock in (KI) of the T-cell receptor KO (CD3−) cells for CAR insertion with AAV addition either before (−12 h), during (0 h), or after (12 h) LNP addition. All results are at day 5 after transfection via LNP.

FIG. 27 provides a table summarizing the number and percent of CD3− cells and KI of the TCR KO (CD3−) cells for CAR insertion with AAV addition at varying doses of LNP 336 (0, 1.0, 2.5, 5.0 μg/mL) and AAV (0K, 5K, 25K, 125K MOI). All results are at day 10 after transfection via LNP.

FIG. 28 provides a table summarizing LNP formulations prepared using SS-OP as the cationic lipid, and their TCR knockout (i.e., CD3−) efficiencies observed on day 4 and day 7 post-transfection with nuclease-encoding mRNA.

FIGS. 29A-29D illustrate flow cytometry analysis of a knockout of the endogenous TCR (i.e., CD3−) following MC3 LNP transfections. The nuclease-encoding mRNA comprised either unmodified UTP or pseudouridine (Pseudo UTP). A) TCR knockout on day 4 following MC3 LNP transfection of mRNA comprising Pseudo UTP. B) TCR knockout on day 7 following MC3 LNP transfection of mRNA comprising Pseudo UTP. C) TCR knockout on day 4 following MC3 LNP transfection of mRNA comprising unmodified UTP. D) TCR knockout on day 7 following MC3 LNP transfection of mRNA comprising unmodified UTP.

FIGS. 30A-30H illustrate flow cytometry analysis of a knockout of the endogenous TCR (i.e., CD3−), and knock-in of a CAR transgene into the TCR locus, on day 4 following MC3 LNP transfections in the presence of various concentrations of human serum. A) TCR knockout and CAR knock-in in the presence of 5% human serum (vol/vol). B) TCR knockout and CAR knock-in in the presence of 2.5% human serum (vol/vol). C) TCR knockout and CAR knock-in in the presence of 1.25% human serum (vol/vol). D) TCR knockout and CAR knock-in in the presence of 0.625% human serum (vol/vol). E) TCR knockout and CAR knock-in in the presence of 0.31% human serum (vol/vol). F) TCR knockout and CAR knock-in in absence of human serum (vol/vol). G) Table summarizing TCR knockout and CAR knock-in, cell counts, and mean fluorescence intensity. H) Tables summarizing the percent TCR knockout, total number of cells with TCR knockout, and total number of cells on day 3 and day 7 after introduction of the nuclease mRNA by LNP.

FIGS. 31A-31H illustrate flow cytometry analysis of knockout of the endogenous TCR (i.e., CD3−) following MC3 LNP transfections in the presence or absence of different ApoE isoforms. A) No ApoE present. B) ApoE2. C) ApoE3. D) ApoE4. E) ApoE2 and ApoE3. F) ApoE3 and ApoE4. G) ApoE2 and ApoE4. H) ApoE2, ApoE3, and ApoE4.

FIGS. 32A and 32B show tables summarizing flow cytometry analysis of knockout of the endogenous TCR (i.e., CD3−), and total TCR-negative cell numbers, following transfection of primary T cells in the presence or absence of different concentrations of ApoE and MC3 LNPs. Frequency of knockout is shown in FIG. 32A, and total numbers of knocked-out cells are shown in FIG. 32B.

### BRIEF DESCRIPTION OF THE SEQUENCES

SEQ ID NO: 1 sets forth the amino acid sequence of the wild-type I-CreI meganuclease from Chlamydomonas reinhardtii.

SEQ ID NO: 2 sets for the amino acid sequence of the TRC 1-2L.1592 meganuclease.

SEQ ID NO: 3 sets for the nucleic acid sequence of the TRC 1-2 recognition sequence (sense) for the TRC 1-2L.1592 meganuclease.

SEQ ID NO: 4 sets for the nucleic acid sequence of the TRC 1-2 recognition sequence (antisense) for the TRC 1-2L.1592 meganuclease.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference. All publications, patent applications, patents, and other references mentioned herein are incorporated by reference herein in their entirety.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “nuclease” or “endonuclease” refers to enzymes which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be, for example, an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in cells, particularly in human immune cells (e.g., T cells), such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to residues 154-195 of SEQ ID NO: 2. In some embodiments, a linker may have an amino acid sequence comprising residues 154-195 of SEQ ID NO: 2.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, 51 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The term “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, EndI, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue): W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific homing endonuclease.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a CpfI CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “altered specificity,” when referencing to a nuclease, means that a nuclease binds to and cleaves a recognition sequence, which is not bound to and cleaved by a reference nuclease (e.g., a wild-type) under physiological conditions, or that the rate of cleavage of a recognition sequence is increased or decreased by a biologically significant amount (e.g., at least 2×, or 2×-10×) relative to a reference nuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template (i.e., a template nucleic acid) into a gene can result in no expression of an encoded protein, expression of a truncated protein, and/or expression of a protein that does not retain its wild-type function. Additionally, introduction of a donor template into a gene can result in no expression of an encoded protein, expression of a truncated protein, and/or expression of a protein that does not retain its wild-type function.

As used herein, “detectable cell-surface expression of an endogenous TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell-surface TCR complex, such as CD3. Methods for detecting cell-surface expression of an endogenous TCR (e.g., an alpha/beta TCR) on an immune cell include those described in the examples herein, and, for example, those described in MacLeod et al. (2017) Molecular Therapy 25(4): 949-961. Cells described herein having no detectable cell-surface expression of an endogenous protein are, therefore, cells in which an endogenous protein such as an endogenous TCR cannot be detected on the cell-surface by such methods.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety and an intracellular domain that comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In various embodiments, the extracellular ligand-binding domain is specific for any antigen or epitope of interest. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the immune effector cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6. Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRIIIa receptor or IgG1. In certain examples, the hinge region can be a CD8 alpha domain.

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest.

As used herein, the terms “T cell receptor alpha gene” or “TCR alpha gene” refer to the locus in a T cell which encodes the T cell receptor alpha subunit. The T cell receptor alpha gene can refer to NCBI Gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region gene” or “TCR alpha constant region gene” or “TRAC” refers to the coding sequence of the T cell receptor alpha gene. The TCR alpha constant region includes the wild-type sequence, and functional variants thereof, identified by NCBI Gen ID NO. 28755.

As used herein, the terms “human beta-2 microglobulin gene,” “B2M gene,” and the like, are used interchangeably and refer to the human gene identified by NCBI Gene ID NO. 567 (Accession No. NG_012920.1), and functional variants thereof.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention.

In some embodiments, a “vector” also refers to a viral vector (i.e., a recombinant virus). Viral vectors can include, without limitation, retroviral vectors (i.e., recombinant retroviruses), lentiviral vectors (i.e., recombinant lentiviruses), adenoviral vectors (i.e., recombinant adenoviruses), and adeno-associated viral (AAV) vectors (i.e., recombinant AAVs).

As used herein, the term “immune cells” refers to cells isolated from a donor, particularly a human donor, which are known to mediate immune responses in the body. Immune cells can include, without limitation, T cells, such as CD4+ and CD8+ T cells, natural killer (NK) cells, B cells, gamma/delta T cells, regulatory T cells, granulocytes, mast cells, monocytes, neutrophils, and dendritic cells.

As used herein, the term “human T cell” or “T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, the term “human B cell” or “B cell” refers to a B cell isolated from a donor, particularly a human donor. B cells, and cells derived therefrom, include isolated B cells that have not been passaged in culture, B cells that have been passaged and maintained under cell culture conditions without immortalization, and B cells that have been immortalized and can be maintained under cell culture conditions indefinitely.

As used herein, the term “immune cell targeting molecule” refers to molecules that selectively bind to molecules on the cell surface of immune cells. Such immune cell targeting molecules can be attached to, anchored to, or otherwise incorporated into or on the surface of lipid nanoparticles in order to selectively bind the lipid nanoparticles to immune cells. Immune cell targeting molecules can include any peptides, nucleic acid molecules, or chemical compounds that selectively bind (i.e., have specificity for) molecules on the cell surface of immune cells including, without limitation, antibodies, antibody fragments (e.g., single-chain variable fragments (scFvs), single-domain antibodies (sdAbs)), dual-affinity re-targeting antibodies (DARTs), aptamers, and the like. For example, a T cell targeting molecule has specificity for a molecule found on the cell surface of a T cell, thus enhancing the binding of a lipid nanoparticle comprising the T cell targeting molecule to a T cell. This term does not embrace apolipoproteins.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the term “5′ cap” refers to a specially altered nucleotide on the 5′ end of primary transcripts such as messenger RNA. 5′ caps of mRNAs are important for RNA stability and processing, mRNA metabolism, the processing and maturation of an RNA transcript in the nucleus, transport of mRNA from the nucleus to the cytoplasm, mRNA stability, and efficient translation of mRNA to protein. A 5′ cap can be a naturally-occurring 5′ cap or one that differs from a naturally-occurring cap of an mRNA. 5′ caps useful for the disclosed method can include any 5′ caps known in the art.

As used herein, the term “nucleoside substitution” refers to the substitution of one or more naturally-occurring nucleosides of an mRNA to a modified nucleoside. Modified nucleosides useful for such substitutions are known in the art.

As used herein, the terms “treatment” or “treating a subject” refers to the administration of a genetically-modified immune cell or population of genetically-modified immune cells of the invention to a subject having a disease, disorder, or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, a genetically-modified immune cell or population of genetically-modified immune cells described herein is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of a genetically-modified immune cell or population of genetically-modified immune cells of the invention, or pharmaceutical compositions disclosed herein, reduces at least one symptom of a disease in a subject. In those embodiments wherein the disease is a cancer, an effective amount of the genetically-modified immune cells or pharmaceutical compositions disclosed herein reduces the level of proliferation or metastasis of cancer, causes a partial or full response or remission of cancer, or reduces at least one symptom of cancer in a subject.

As used herein, the term “cancer” should be understood to encompass any neoplastic disease (whether invasive or metastatic) which is characterized by abnormal and uncontrolled cell division causing malignant growth or tumor.

As used herein, the term “carcinoma” refers to a malignant growth made up of epithelial cells.

As used herein, the term “leukemia” refers to malignancies of the hematopoietic organs/systems and is generally characterized by an abnormal proliferation and development of leukocytes and their precursors in the blood and bone marrow.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “melanoma” refers to a tumor arising from the melanocytic system of the skin and other organs.

As used herein, the term “lymphoma” refers to a group of blood cell tumors that develop from lymphocytes.

As used herein, the term “blastoma” refers to a type of cancer that is caused by malignancies in precursor cells or blasts (immature or embryonic tissue).

As used herein, the phrase “lipid nanoparticle” refers to a microscopic lipid formulation that can be used to deliver an active agent or therapeutic agent, such as a nucleic acid (e.g., an mRNA), to a target site of interest (e.g., an immune cell).

As used herein, the phrase “lipid formulation” refers to a formulation comprising one or more lipids (e.g., cationic lipids, non-cationic lipids, lipid conjugates, and the like).

As used herein, the term “lipid” refers to a group of organic compounds that include, but are not limited to, esters of fatty acids and are characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids. The selection of the individual lipid components of the lipid formulation is made to optimize delivery of an mRNA to the target cell.

As used herein, the term “steroid” refers to a class of hydrophobic, biologically active compounds comprising a specific 17-carbon fused ring system having three six membered rings and one five membered ring (a cyclopentanoperhydrophenanthrene ring system).

The term “lipid conjugate” refers to a conjugated lipid that inhibits aggregation of lipid particles.

As used herein, the term “zeta potential” refers to the overall charge that a nanoparticle acquires in a particular medium, and is a measure of electrostatic attraction and repulsion. Zeta potential values are indicative of dispersion stability, aggregation, and diffusion behavior. Zeta potential may be calculated from electrokinetic data obtained from, e.g., laser Doppler velocimetry. In this technique, a voltage is applied across a pair of electrodes at either end of a cell containing a nanoparticle dispersion. Charged nanoparticles are attracted to the oppositely charged electrode, and their velocity is measured and expressed in unit field strength as their electrophoretic mobility. Zeta values may be predictive in determining penetration through various cellular membranes.

As used herein, the term “polydispersity index” or “PDI” refers to the distribution of nanoparticle size and is a measure of uniformity. The polydispersity index is a unit-less measure which may be calculated from particle size data obtained according to techniques known in the art, for example, dynamic light scattering. Smaller values indicate a narrower size distribution, i.e., a more consistent particle size.

As used herein, the term “serum-free” refers to the use of liquid, solid, or liquid and solid culture media that lacks or is substantially free from serum (e.g., fetal bovine serum, calf bovine serum) for the growth of cells in culture.

As used herein, the term “exogenous” or “heterologous” in reference to a polynucleotide or nucleotide sequence is intended to mean a polynucleotide or sequence that is purely synthetic, that originates from a foreign species, or, if from the same species, is substantially modified from its native form in composition and/or genomic locus by deliberate human intervention.

The term “effector function” refers to a specialized function of a cell. Effector function of a T cell, for example, may be cytolytic activity or helper activity including the secretion of cytokines.

As used herein, the term “non-cationic lipid” refers to any neutral, zwitterionic, or anionic lipid.

As used herein, the term “anionic lipid” refers to any of a number of lipid species that carry a net negative charge at a selected pH, such as physiological pH.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values of from about 0 to 2 can take the values 0, 1 or 2 if the variable is inherently discrete, and can take the values 0.0, 0.1, 0.01, 0.001, or any other real values 0 and 2 if the variable is inherently continuous.

### 2.1 Principle of the Invention

Without wishing to be bound by any particular theory, it has been discovered according to the present disclosure that use of certain lipid nanoparticles, in the presence of an apolipoprotein, can be used effectively for the delivery of nuclease mRNA into immune cells, resulting in genetic modification of such immune cells, while preventing several negative impacts typically associated with mRNA delivery by electroporation.

Thus, provided herein is a method for preparing genetically-modified immune cells, wherein the method comprises contacting immune cells with lipid nanoparticles in the presence of an apolipoprotein. For example, the immune cells and the lipid nanoparticles can be contacted within a composition comprising the apolipoprotein. Addition of an apolipoprotein allows for an increase, sometimes 2-fold to 3-fold higher, in the resulting gene editing frequency and/or frequency of transgene insertion, in the immune cells. The lipid nanoparticles described herein can comprise, for example, a cationic lipid selected from DLin-DMA (1,2-dilinoleyloxy-3-dimethylaminopropane), DLin-MC3-DMA (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9-octadecenoyloxy)phenylacetoxy]ethyl}piperidinyl)ethyl] disulfide), and derivatives thereof. Further, the lipid nanoparticles comprise mRNA encoding an engineered nuclease having specificity for a recognition sequence in the genome of the immune cells. Contacting the immune cells with the lipid nanoparticles results in the delivery of the engineered nuclease-encoding mRNA into the immune cells where the engineered nuclease is expressed. Subsequently, the engineered nuclease generates a cleavage site at the recognition sequence to generate a genetically-modified immune cell. Such genetically-modified immune cell can be, for example, T cells, NK cell, or B cells. Moreover, such cells can be further modified to express a CAR or exogenous TCR, either by random integration of a coding sequence in the cell genome, or by targeted insertion of a coding sequence into the nuclease cleavage site. Specific embodiments of the invention are described in detail herein below.

### 2.2 Lipid Nanoparticles

Methods for preparing genetically-modified immune cells as disclosed herein include contacting immune cells, such as primary immune cells, with lipid nanoparticles in the presence of an apolipoprotein. For example, the immune cells and the lipid nanoparticles can be contacted within a composition comprising the apolipoprotein. A major characteristic of lipid nanoparticles is the fact that they are prepared with physiologically well-tolerated lipids.

The lipid nanoparticles described herein for delivery of nuclease mRNA to an immune cell comprise a cationic lipid selected from DLin-DMA, DLin-MC3-DMA, DLin-KC2-DMA, DODMA, SS-OP, and derivatives thereof. DLin-MC3-DMA and derivatives thereof are described, for example, in WO 2010144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, N.Y.) is described, for example, at www.nofamerica.com/store/index.php?dispatch=products.view&product_id=962.

Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(α-D-tocopherolhemisuccinatepropyl)amino]ethyl} disulfide (SS-33/3APO5), Bis{2-[4-(α-D-tocopherolhemisuccinateethyl)piperidyl]ethyl} disulfide (SS33/4PE15), Bis{2-[4-(cis-9-octadecenoateethyl)-1-piperidinyl]ethyl} disulfide (SS18/4PE16), and Bis{2-[4-(cis,cis-9,12-octadecadienoateethyl)-1-piperidinyl]ethyl} disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In some of these embodiments, the molar concentration of the cationic lipid is about 40% of the total lipid molar concentration. In some of these embodiments, the molar concentration of the cationic lipid is about 48.5% of the total lipid molar concentration. In some of these embodiments, the molar concentration of the cationic lipid is about 50% of the total lipid molar concentration. In some of these embodiments, the molar concentration of the cationic lipid is about 60% of the total lipid molar concentration. In certain embodiments, the lipid nanoparticles comprise a molar ratio of cationic lipid to mRNA of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20. In some of these embodiments, the lipid nanoparticles comprise a molar ratio of cationic lipid to mRNA of about 8.

The lipid nanoparticles utilized in the presently disclosed methods can comprise at least one non-cationic lipid.

In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. In some of these embodiments, the molar concentration of the non-cationic lipids is about 40% of the total lipid molar concentration. In some of these embodiments, the molar concentration of the non-cationic lipids is about 48.5% of the total lipid molar concentration. In some of these embodiments, the molar concentration of the non-cationic lipids is about 50% of the total lipid molar concentration. In some of these embodiments, the molar concentration of the non-cationic lipids is about 60% of the total lipid molar concentration.

Non-cationic lipids include, in some embodiments, phospholipids and steroids. Phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate (Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate (Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol . . . )(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine (Sodium Salt) (DLPS-NA), 1,2-Dimyristoyl-sn-glycero-3-phosphate (Sodium Salt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine (Sodium Salt) (DMPS-NA), 1,2-Dioleoyl-sn-glycero-3-phosphate (Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine (Sodium Salt) (DOPS-NA), 1,2-Dipalmitoyl-sn-glycero-3-phosphate (Sodium Salt) (DPPA-NA), 1,2-Dipalmitoyl-sn-glycero-3-phosphocholine (DPPC) 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine (Sodium Salt) (DPPS-NA), 1,2-Distearoyl-sn-glycero-3-phosphate (Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine (Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LYSOPCMYRISTIC), 1-Palmitoyl-sn-glycero-3-phosphocholine (LYS OPCPALMITIC), 1-Stearoyl-sn-glycero-3-phosphocholine (LYS OPCSTEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the molar concentration of the phospholipid is from about 0% to about 30%, from about 2.5% to about 25%, from about 5% to about 20%, from about 5% to about 15%, from about 7.5% to about 12.5%, or about 1%, about 2%, about 5%, about 10%, about 15%, about 20%, about 25%, or about 30% of the total lipid molar concentration. In particular embodiments, the molar concentration of the phospholipid is about 5% of the total lipid molar concentration. In particular embodiments, the molar concentration of the phospholipid is about 10% of the total lipid molar concentration. In particular embodiments, the molar concentration of the phospholipid is about 18% of the total lipid molar concentration. In particular embodiments, the molar concentration of the phospholipid is about 20% of the total lipid molar concentration. In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some of these embodiments, the molar concentration of the steroid is from about 20% to about 60%, from about 25% to about 55%, from about 30% to about 50%, from about 35% to about 40%, about 20%, about 25%, about 30%, about 35%, about 38.5%, about 40%, about 45%, about 50%, about 55%, or about 60% of the total lipid molar concentration. In particular embodiments, the molar concentration of the steroid is about 30% of the total lipid molar concentration. In some of these embodiments, the molar concentration of the steroid is about 38.5% of the total lipid molar concentration. In particular embodiments, the molar concentration of the steroid is about 50% of the total lipid molar concentration. In some embodiments of the presently disclosed methods, the lipid nanoparticles used for delivering mRNA encoding an engineered nuclease comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000 PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

In certain embodiments, the molar concentration of the lipid conjugate is from about 0.01% to about 10%, from about 0.2% to about 8%, from about 0.5% to about 5%, from about 0.1% to about 1.5%, from about 1% to about 2%, about 0.01%, about 0.05%, about 0.1%, about 0.15%, about 0.2%, about 0.5%, about 0.75%, about 1%, about 1.2%, about 1.5%, about 2%, about 2.5%, about 3%, about 4%, about 5%, about 6%, about 7%, about 8%, about 9%, or about 10% of the total lipid molar concentration. In some of these embodiments, the molar concentration of the lipid conjugate is about 1.5% of the total lipid molar concentration.

In some embodiments, the lipid nanoparticle compositions described herein include a cationic lipid that is DLin-MC3-DMA, a steroid that is cholesterol, a phospholipid that is DSPC, and a lipid conjugate that is PEG 5000. In some other embodiments, the lipid nanoparticle compositions described herein include a cationic lipid that is DLin-MC3-DMA, a steroid that is cholesterol, a phospholipid that is DSPC, and a lipid conjugate that is PEG 2000. In some other embodiments, the lipid nanoparticle compositions described herein include a cationic lipid that is DLin-MC3-DMA, a steroid that is cholesterol, a phospholipid that is DOPC, and a lipid conjugate that is PEG 2000.

In some embodiments, a molar ratio of the cationic lipid to the phospholipid is from about 1:1 to about 20:1, about 2:1 to about 20:1, about 3:1 to about 20:1, about 4:1 to about 20:1, about 6:1 to about 20:1, about 8:1 to about 20:1, about 10:1 to about 20:1, about 12:1 to about 20:1, about 14:1 to about 20:1, about 16:1 to about 20:1, about 18:1 to about 20:1, or about 2:1 to about 7:1. In particular embodiments, the molar ratio of the cationic lipid to the phospholipid is from about 2:1 to about 7:1. In further embodiments, the molar ratio of the cationic lipid to the phospholipid is about 2:1, about 4:1, about 5:1, or about 6:1.

In some embodiments, a molar ratio of the cationic lipid to the steroid is from about 0.25:1 to about 5:1, about 0.5:1 to about 5:1, about 0.75:1 to about 5:1, about 1:1 to about 5:1, about 2:1 to about 5:1, about 3:1 to about 5:1, about 4:1 to about 5:1, about 0.5:1 to about 3:1, or about 0.8:1 to about 2:1. In some embodiments, the molar ratio of the cationic lipid to the steroid is from about 0.8:1 to about 2:1. In some embodiments, the molar ratio of the cationic lipid to the steroid is about 0.8:1, about 1.3:1, about 1:1, or about 2:1.

In some embodiments, a molar ratio of the cationic lipid to the lipid conjugate is from about 10:1 to about 1000:1, about 25:1 to about 1000:1, about 50:1 to about 1000:1, about 75:1 to about 1000:1, about 100:1 to about 1000:1, about 250:1 to about 1000:1, about 400:1 to about 1000:1, about 550:1 to about 1000:1, about 700:1 to about 1000:1, about 850:1 to about 1000:1, about 20:1 to about 600:1, or about 25:1 to about 400:1. In some embodiments, the molar ratio of the cationic lipid to the lipid conjugate is from about 25:1 to about 400:1. In some embodiments, the molar ratio of the cationic lipid to the lipid conjugate is about 25:1, about 33:1, about 60:1, or about 400:1.

In some embodiments, a molar ratio of the steroid to the lipid conjugate is from about 5:1 to about 750:1, about 25:1 to about 750:1, about 50:1 to about 750:1, about 75:1 to about 750:1, about 100:1 to about 750:1, about 150:1 to about 750:1, about 200:1 to about 750:1, about 250:1 to about 750:1, about 300:1 to about 750:1, about 350:1 to about 750:1, about 400:1 to about 750:1, about 450:1 to about 750:1, about 500:1 to about 750:1, about 550:1 to about 750:1, about 600:1 to about 750:1, about 650:1 to about 750:1, about 700:1 to about 750:1, about 10:1 to about 500:1, or about 25:1 to about 500:1. In some embodiments, the molar ratio of the steroid to the lipid conjugate is from about 25:1 to about 500:1. In some embodiments, the molar ratio of the steroid to the lipid conjugate is from about 25:1, about 30:1, or about 500:1.

In some embodiments, a molar ratio of the phospholipid to the lipid conjugate is from about 1:1 to about 300:1, about 5:1 to about 300:1, about 10:1 to about 300:1, about 15:1 to about 300:1, about 20:1 to about 300:1, about 25:1 to about 300:1, about 50:1 to about 300:1, about 75:1 to about 300:1, about 100:1 to about 300:1, about 125:1 to about 300:1, about 150:1 to about 300:1, about 175:1 to about 300:1, about 200:1 to about 300:1, about 225:1 to about 300:1, about 250:1 to about 300:1, about 275:1 to about 300:1, about 3:1 to about 200:1, or about 5:1 to about 100:1. In some embodiments, the molar ratio of the phospholipid to the lipid conjugate is from about 5:1 to about 100:1. In some embodiments, the molar ratio of the phospholipid to the lipid conjugate is about 6:1, about 10:1, about 13:1 or about 100:1.

In particular embodiments, the lipid nanoparticles comprise DLin-MC3-DMA, DSPC, cholesterol, and DMG-PEG2000 at a molar ratio of about 50:10:38.5:1.5. In particular embodiments, the lipid nanoparticles comprise DLin-MC3-DMA, DSPC, cholesterol, and DMG-PEG2000 at a molar ratio of about 40:10:48.5:1.50. In other particular embodiments, the lipid nanoparticles comprise DODMA, DSPC, cholesterol, and DMG-PEG2000 at a molar ratio of about 50:10:38.5:1.5. In further embodiments, the lipid nanoparticles comprise DLin-MC3-DMA, DOPC, cholesterol, and DMG-PEG5000 at a molar ratio of about 40:10:49.90:0.10. In some embodiments, the lipid nanoparticles comprise DLin-MC3-DMA, DOPC, cholesterol, and DMG-PEG2000 at a molar ratio of about 40:20:38.5:1.5. In some embodiments, the lipid nanoparticles comprise DLin-MC3-DMA, DOPC, cholesterol, and DMG-PEG2000 at a molar ratio of about 60:10:29:1.

In some embodiments, the lipid nanoparticle compositions described herein can be any one of the compositions according to Table 1 below.

TABLE 1
Lipid Nanoparticle Compositions According to the Invention

| | | | | | | Phos. | | Lpd. |
|---|---|---|---|---|---|---|---|---|
| | | Cat. | | Steroid | Phos. | Lpd | | Conj.. |
| No. | Cat. Lpd. | Lpd. % | Steroid | % | Lpd. | Amnt. % | Lpd Conj, | % |
| 1 | DODMA | 50 | Cholst. | 38.50 | DSPC | 10 | DMG PEG | 1.50 |
| 2 | SS-33/3APO5 | 50 | Cholst | 40 | DSPC | 8 | DMG PEG | 2.50 |
| 3 | DODMA | 50 | Cholst | 38.50 | DSPC | 10 | DMG PEG | 1.50 |
| 4 | SS-33/3APO5 | 50 | Cholst | 40 | DSPC | 8 | DMG PEG | 2.50 |
| 5 | Dlin-MC3-DMA | 50 | Cholst | 38.50 | DSPC | 10 | DMG PEG | 1.50 |
| 265 | Dlin-MC3-DMA | 60 | Cholst | 28.50 | DSPC | 10 | DMG PEG 5000 | 1.50 |
| 266 | Dlin-MC3-DMA | 40 | Cholst | 49.90 | DSPC | 10 | DMG PEG 5000 | 0.10 |
| 267 | Dlin-MC3-DMA | 52 | Cholst | 27 | DOPC | 20 | DMG PEG 5000 | 1 |
| 269 | Dlin-MC3-DMA | 58.50 | Cholst | 20 | DSPC | 20 | DMG PEG 2000 | 1.50 |
| 271 | Dlin-MC3-DMA | 50 | Cholst | 34.90 | DOPC | 15 | DMG PEG 2000 | 0.10 |
| 272 | Dlin-MC3-DMA | 60 | Cholst | 29 | DOPC | 10 | DMG PEG 2000 | 1 |
| 273 | Dlin-MC3-DMA | 40 | Cholst | 38.50 | DOPC | 20 | DMG PEG 2000 | 1.50 |
| 275 | Dlin-MC3-DMA | 54 | Cholst | 34.50 | DOPC | 10 | DMG PEG 2000 | 1.50 |
| 276 | Dlin-MC3-DMA | 40 | Cholst | 38.50 | DSPC | 20 | DMG PEG 5000 | 1.50 |
| 277 | Dlin-MC3-DMA | 40 | Cholst | 39.90 | DOPC | 20 | DMG PEG 5000 | 0.10 |
| 279 | Dlin-MC3-DMA | 40 | Cholst | 48.50 | DSPC | 10 | DMG PEG 2000 | 1.50 |
| 280 | Dlin-MC3-DMA | 40 | Cholst | 48.50 | DOPC | 10 | DMG PEG 5000 | 1.50 |
| 281 | Dlin-MC3-DMA | 60 | Cholst | 20.50 | DOPC | 18 | DMG PEG 5000 | 1.50 |
| 282 | Dlin-MC3-DMA | 59.90 | Cholst | 20 | DSPC | 20 | DMG PEG 5000 | 0.10 |
| 284 | Dlin-MC3-DMA | 60 | Cholst | 24 | DSPC | 15 | DMG PEG 2000 | 1 |
| 285 | Dlin-MC3-DMA | 50 | Cholst | 38.50 | DSPC | 10 | DMG PEG 2000 | 1.50 |
| 293 | Dlin-MC3-DMA | 50 | Cholst | 43.50 | DOPE | 5 | DMG PEG 5000 | 1.50 |
| 298 | Dlin-MC3-DMA | 34.50 | Cholst | 59.50 | DOPE | 5 | DMG PEG 5000 | 1 |
| 299 | Dlin-MC3-DMA | 44 | Cholst | 40 | DOPE | 15 | DMG PEG 5000 | 1 |
| 302 | Dlin-MC3-DMA | 44 | Cholst | 40 | DOPE | 15 | DMG PEG 2000 | 1 |
| 303 | Dlin-MC3-DMA | 30 | Cholst | 53.50 | DOPE | 15 | DMG PEG 5000 | 1.50 |
| 304 | Dlin-MC3-DMA | 39 | Cholst | 49.50 | DOPE | 10 | DMG PEG 2000 | 1.50 |
| 306 | Dlin-MC3-DMA | 33.50 | Cholst | 60 | DOPE | 5 | DMG PEG 2000 | 1.50 |
| 310 | Dlin-MC3-DMA | 30 | Cholst | 60 | DOPE | 9 | DMG PEG 5000 | 1 |
| 311 | Dlin-MC3-DMA | 40 | Cholst | 48.50 | DSPC | 10 | DMG PEG 2000 | 1.50 |
| 312 | Dlin-MC3-DMA | 50 | Cholst | 38.50 | DSPC | 10 | DMG PEG 2000 | 1.50 |
| 314 | Dlin-MC3-DMA | 40 | Cholst | 49.50 | DSPC | 10 | DMG PEG 5000 | 0.50 |
| 315 | Dlin-MC3-DMA | 50 | Cholst | 38.50 | DSPC | 10 | DMG PEG 2000 | 1.50 |
| 316 | MPDACA | 50 | Cholst | 38.50 | DSPC | 10 | DMG PEG 2000 | 1.50 |
| 317 | SS33/4PE15 | 50 | Cholst | 38.50 | DSPC | 10 | DMG PEG 2000 | 1.50 |
| 318 | SS33/3APO5 | 50 | Cholst | 38.50 | DSPC | 10 | DMG PEG 2000 | 1.50 |
| 319 | SS18/4PE16 | 50 | Cholst | 38.50 | DSPC | 10 | DMG PEG 2000 | 1.50 |
| 320 | SS18/4PE13 | 50 | Cholst | 38.50 | DSPC | 10 | DMG PEG 2000 | 1.50 |
| 321 | PONA | 50 | Cholst | 38.50 | DSPC | 10 | DMG PEG 2000 | 1.50 |
| 322 | GUADACA | 50 | Cholst | 38.50 | DSPC | 10 | DMG PEG 2000 | 1.50 |
| 323 | DOTMA | 50 | Cholst | 38.50 | DSPC | 10 | DMG PEG 2000 | 1.50 |
| 324 | DODMA | 50 | Cholst | 38.50 | DSPC | 10 | DMG PEG 2000 | 1.50 |
| 325 | DOTAP | 50 | Cholst | 38.50 | DSPC | 10 | DMG PEG 2000 | 1.50 |
| 336 | Dlin-MC3-DMA | 40 | Cholst | 48.50 | DSPC | 10 | DMG PEG 2000 | 1.50 |
| 337 | Dlin-MC3-DMA | 40 | Cholst | 48.50 | DSPC | 10 | DMG PEG 2000 | 1.50 |
| 338 | Dlin-KC2-DMA | 40 | Cholst | 48.50 | DSPC | 10 | DMG PEG 2000 | 1.50 |
| 339 | DlinDMA | 40 | Cholst | 48.50 | DSPC | 10 | DMG PEG 2000 | 1.50 |
| 340 | Dlin-MC3-DMA | 50 | Cholst | 38.50 | DSPC | 10 | DMG PEG 2000 | 1.50 |
| 341 | Dlin-MC3-DMA | 50 | Cholst | 38.50 | DSPC | 10 | DMG PEG 2000 | 1.50 |
| 342 | Dlin-KC2-DMA | 50 | Cholst | 38.50 | DSPC | 10 | DMG PEG 2000 | 1.50 |
| 343 | DlinDMA | 50 | Cholst | 38.50 | DSPC | 10 | DMG PEG 2000 | 1.50 |
| 358 | SS-OP | 40 | Cholst | 48.5 | DSPC | 10 | DMG PEG 2000 | 1.50 |
| 360 | SS-OP | 60 | Cholst | 38.5 | DSPC | 10 | DMG PEG 2000 | 1.50 |
| 361 | SS-OP | 52.5 | Cholst | 40.0 | DSPC | 7.5 | DMG PEG 2000 | 1.50 |
| 362 | Dlin-MC3-DMA | 40 | Cholst | 48.50 | DSPC | 10 | DMG PEG 2000 | 1.50 |
| 363 | Dlin-MC3-DMA | 40 | Cholst | 48.50 | DSPC | 10 | DMG PEG 2000 | 1.50 |

The selection of cationic lipids, non-cationic lipids and/or lipid conjugates which comprise the lipid nanoparticle, as well as the relative molar ratio of such lipids to each other, is based upon the characteristics of the selected lipid(s), the nature of the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S. Patent Publication Nos. 20040142025 and 20070042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421\hat{ }150 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

In some embodiments, the polydispersity index of the lipid nanoparticles is less than about 0.3, or less than about 0.2.

In some embodiments, the zeta potential of the lipid nanoparticles is from about −40 mV to about 40 mV, or from about −10 mV to about 10 mV.

Given the efficiency of the presently disclosed methods for delivering the mRNA payload of the lipid nanoparticles, cell targeting molecules (e.g., T cell targeting molecules) on the surface of the lipid nanoparticles are not necessary. Thus, in some embodiments, the lipid nanoparticles do not comprise an immune cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to immune cells.

### 2.3 Apolipoproteins

According to the present disclosure, it has surprisingly been found that methods for delivering nuclease mRNA via lipid nanoparticles, and generating genetically-modified immune cells, is enhanced by the inclusion of an apolipoprotein. Apolipoproteins are proteins that bind to and assist in solubilizing hydrophobic lipids and aiding in their transport. Apolipoproteins possess amphipathic (detergent-like) properties, and surround hydrophobic lipids to create a lipoprotein particle that is water soluble. Apolipoproteins are components of different lipoproteins and can be defined as non-exchangeable or exchangeable. For example, ApoB is non-exchangeable and anchored in the lipoprotein particle, whereas apoA1, ApoE, ApoD, ApoJ, ApoH, and ApoM are exchangeable and can be transferred between different lipoprotein particles.

In some embodiments, the apolipoprotein used in the presently disclosed methods is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), apolipoprotein (a) (Apo(a)) protein, or a combination thereof. In some of these embodiments, the apolipoprotein is ApoE. ApoE can be any isoform of ApoE, including, for example, ApoE2, ApoE3, and ApoE4. In particular embodiments, the apolipoprotein is present at a concentration between about 0.01 μg/mL to about 10 μg/mL, about 0.1 μg/mL to about 5 μg/mL, about 0.5 μg/mL to about 2 μg/mL, or about 1 μg/mL. In some embodiments, the apolipoprotein is present at a concentration of about 1 μg/mL. In certain embodiments, the apolipoprotein is present at a concentration of about 0.9 μg/mL. In certain embodiments, the apolipoprotein is present at a concentration of about 0.8 μg/mL. In certain embodiments, the apolipoprotein is present at a concentration of about 0.7 μg/mL. In certain embodiments, the apolipoprotein is present at a concentration of about 0.6 μg/mL. In certain embodiments, the apolipoprotein is present at a concentration of about 0.5 μg/mL. In certain embodiments, the apolipoprotein is present at a concentration of about 0.4 μg/mL. In certain embodiments, the apolipoprotein is present at a concentration of about 0.3 μg/mL. In certain embodiments, the apolipoprotein is present at a concentration of about 0.2 μg/mL. In certain embodiments, the apolipoprotein is present at a concentration of about 0.1 μg/mL. In certain embodiments, the apolipoprotein is present at a concentration of about 1.1 μg/mL. In certain embodiments, the apolipoprotein is present at a concentration of about 1.2 μg/mL. In certain embodiments, the apolipoprotein is present at a concentration of about 1.3 μg/mL. In certain embodiments, the apolipoprotein is present at a concentration of about 1.4 μg/mL. In certain embodiments, the apolipoprotein is present at a concentration of about 1.5 μg/mL. In certain embodiments, the apolipoprotein is present at a concentration of about 1.6 μg/mL. In certain embodiments, the apolipoprotein is present at a concentration of about 1.7 μg/mL. In certain embodiments, the apolipoprotein is present at a concentration of about 1.8 μg/mL. In certain embodiments, the apolipoprotein is present at a concentration of about 1.9 μg/mL. In certain embodiments, the apolipoprotein is present at a concentration of about 2.0 μg/mL. Concentrations of apolipoproteins can be considered, for example, to be the amount of the apolipoprotein (e.g., μg) per volume (e.g., mL) of medium in which the immune cells are cultured.

In particular embodiments, the apolipoprotein is apolipoprotein E (ApoE) which is present at a concentration of about 1 μg/mL. In certain embodiments, ApoE is present at a concentration of about 0.9 μg/mL. In certain embodiments, ApoE is present at a concentration of about 0.8 μg/mL. In certain embodiments, ApoE is present at a concentration of about 0.7 μg/mL. In certain embodiments, ApoE is present at a concentration of about 0.6 μg/mL. In certain embodiments, ApoE is present at a concentration of about 0.5 μg/mL. In certain embodiments, ApoE is present at a concentration of about 0.4 μg/mL. In certain embodiments, ApoE is present at a concentration of about 0.3 μg/mL. In certain embodiments, ApoE is present at a concentration of about 0.2 μg/mL. In certain embodiments, ApoE is present at a concentration of about 0.1 μg/mL. In certain embodiments, ApoE is present at a concentration of about 1.1 μg/mL. In certain embodiments, ApoE is present at a concentration of about 1.2 μg/mL. In certain embodiments, ApoE is present at a concentration of about 1.3 μg/mL. In certain embodiments, ApoE is present at a concentration of about 1.4 μg/mL. In certain embodiments, ApoE is present at a concentration of about 1.5 μg/mL. In certain embodiments, ApoE is present at a concentration of about 1.6 μg/mL. In certain embodiments, ApoE is present at a concentration of about 1.7 μg/mL. In certain embodiments, ApoE is present at a concentration of about 1.8 μg/mL. In certain embodiments, ApoE is present at a concentration of about 1.9 μg/mL. In certain embodiments, ApoE is present at a concentration of about 2.0 μg/mL. Concentrations of ApoE can be considered, for example, to be the amount of the ApoE (e.g., μg) per volume (e.g., mL) of medium in which the immune cells are cultured.

### 2.4 Methods for Preparing Populations of Genetically-Modified Immune Cells

As previously stated herein, the present disclosure generally provides a method for preparing genetically-modified immune cells, wherein the method comprises contacting immune cells with lipid nanoparticles in the presence of an apolipoprotein. For example, the immune cells and the lipid nanoparticles can be contacted within a composition comprising the apolipoprotein.

In some embodiments, the immune cells that are genetically-modified using the presently disclosed methods are human immune cells. In some embodiments, the immune cells are T cells, or cells derived therefrom. In other embodiments, the immune cells are natural killer (NK) cells, or cells derived therefrom. In still other embodiments, the immune cells are B cells, or cells derived therefrom.

Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines, NK cell lines, or B cell lines available in the art may be used. In some embodiments of the present disclosure, immune cells are obtained from a unit of blood collected from a subject using any number of techniques known to the skilled artisan. In one embodiment, cells from the circulating blood of an individual are obtained by apheresis.

The immune cells are contacted with the lipid nanoparticles such that the payload of the lipid nanoparticles is delivered into the immune cells. The lipid nanoparticles comprise the mRNA encoding an engineered nuclease. In some embodiments, immune cells are further contacted with lipid nanoparticles comprising a template nucleic acid which comprises an exogenous polynucleotide encoding a polypeptide of interest (e.g., a CAR or exogenous TCR).

In some embodiments, the method is performed in vitro. In some embodiments, the immune cells are contacted with the lipid nanoparticles under serum-free culture conditions (e.g., culture conditions substantially free of serum). In some embodiments, the immune cells are contacted with the lipid nanoparticles in a culture condition comprising a concentration of serum (vol/vol) of less than about 0.31%, less than about 0.625%, less than about 1.25%, less than about 2.5%, less that about 5%, or less than about 10%. In certain embodiments, the immune cells are contacted with the lipid nanoparticles in a culture condition comprising less than about 0.31% serum (vol/vol). In certain embodiments, the immune cells are contacted with the lipid nanoparticles in a culture condition comprising less than about 0.625% serum (vol/vol). In certain embodiments, the immune cells are contacted with the lipid nanoparticles in a culture condition comprising less than about 1.25% serum (vol/vol). In certain embodiments, the immune cells are contacted with the lipid nanoparticles in a culture condition comprising less than about 2.5% serum (vol/vol). In certain embodiments, the immune cells are contacted with the lipid nanoparticles in a culture condition comprising less than about 5% serum (vol/vol). In certain embodiments, the immune cells are contacted with the lipid nanoparticles in a culture condition comprising less than about 10% serum (vol/vol). In certain embodiments, the immune cells are contacted with the lipid nanoparticles in a culture condition comprising serum at a concentration (vol/vol) of from about 0% to about 0.31%, from about 0% to about 0.625%, from about 0% to about 1.25%, from about 0% to about 2.5%, from about 0% to about 5%, or from about 0% to about 10%. In certain embodiments, the immune cells are contacted with the lipid nanoparticles in a culture condition comprising serum at a concentration (vol/vol) of from about 0% to about 0.31%. In certain embodiments, the immune cells are contacted with the lipid nanoparticles in a culture condition comprising serum at a concentration (vol/vol) of from about 0% to about 0.625%. In certain embodiments, the immune cells are contacted with the lipid nanoparticles in a culture condition comprising serum at a concentration (vol/vol) of from about 0% to about 1.25%. In certain embodiments, the immune cells are contacted with the lipid nanoparticles in a culture condition comprising serum at a concentration (vol/vol) of from about 0% to about 2.5%. In certain embodiments, the immune cells are contacted with the lipid nanoparticles in a culture condition comprising serum at a concentration (vol/vol) of from about 0% to about 5%. In certain embodiments, the immune cells are contacted with the lipid nanoparticles in a culture condition comprising serum at a concentration (vol/vol) of from about 0% to about 10%. Concentrations of serum can be considered, for example, to be the volume of serum per volume of medium in which the immune cells are cultured.

The lipid nanoparticles utilized in the presently disclosed methods comprise mRNA encoding an engineered nuclease having specificity for a recognition sequence in the genome of the immune cells. Upon contact with the immune cells and in the presence of an apolipoprotein, the mRNA is delivered into the immune cells and the engineered nuclease is expressed. Upon expression, the engineered nuclease subsequently generates a cleavage site at the recognition sequence. The generation of a cleavage site results in a genetically-modified immune cell. In some examples, a cleavage site in a target gene is repaired by error-prone non-homologous end joining, resulting in disrupted expression of the polypeptide encoded by the gene. In some other examples, an exogenous polynucleotide is inserted into the cleavage site, resulting in disrupted expression of the polypeptide encoded by the gene, and expression of one or more transgenes encoded by the exogenous polynucleotide.

In some embodiments, the engineered nuclease encoded by the mRNA, and which generates the cleavage site in the immune cell genome, is an engineered meganuclease, a zinc finger nuclease, a TALEN, a compact TALEN, a CRISPR system nuclease, or a megaTAL. In certain embodiments, the engineered nuclease is an engineered meganuclease. In particular embodiments, the engineered nuclease used to practice the invention is a single-chain meganuclease.

In some embodiments, the recognition sequence of the engineered nuclease is in a target gene. Expression of a polypeptide encoded by the target gene can be disrupted by non-homologous end joining at the cleavage site. In particular embodiments, the target gene is selected from the group consisting of a TCR alpha gene, a TCR alpha constant region gene, a TCR beta gene, a TCR beta constant region gene, a beta-2 microglobulin gene, a CD52 gene, a CS1 (i.e., SLAMF7 or CD319) gene, a Cbl proto-oncogene B (CBL-B) gene, a CD52 gene, a CD7 gene, a programmed cell death-1 (PD-1) gene, a lymphocyte-activation 3 (LAG-3) gene, a transforming growth factor beta receptor II (TGFBRII) gene, a T-cell immunoglobulin and mucin-domain containing-3 (TIM-3) gene, a T cell immunoreceptor with Ig and ITIM domains (TIGIT) gene, a CD70 gene, a glucocorticoid receptor gene, a Tet methylcytosine dioxygenase 2 (TET2) gene, a general control nonderepressible 2 (GCN2) gene, a deoxycytidine kinase (DCK) gene, a cytotoxic T-lymphocyte associated protein 4 (CTLA-4) gene, or a C-C motif chemokine receptor 5 (CCR5) gene.

In some of these embodiments, the target gene is a TCR alpha constant region gene, and the genetically-modified T cells prepared using the presently disclosed methods therefore do not have detectable cell-surface expression of an endogenous TCR, such as the alpha/beta TCR. In some of these embodiments, the cleavage site is within the first exon of the TCR alpha constant region gene. In particular embodiments, the genetically-modified immune cells express a CAR or exogenous TCR.

In particular embodiments, the immune cells can be contacted with a first population of lipid nanoparticles comprising mRNA encoding a first engineered nuclease having specificity for a first recognition sequence, and simultaneously or subsequently contacted with a second population of lipid nanoparticles comprising mRNA encoding a second engineered nuclease having specificity for a second recognition sequence. In such embodiments of the methods, the first engineered nuclease and the second engineered nuclease are expressed in the immune cells, the first engineered nuclease generates a first cleavage site in the first recognition sequence, and the second engineered nuclease generates a second cleavage site in the second recognition sequence. In some instances, the first recognition sequence and the second recognition sequence are in the same target gene, such that expression of a polypeptide encoded by the target gene is disrupted by non-homologous end joining at the first cleavage site and/or the second cleavage site. In other examples, the first recognition sequence and the second recognition sequence are in different target genes, such that expression of polypeptides encoded by the different target genes is disrupted by non-homologous end joining at the first cleavage site and the second cleavage site. The target gene(s) targeted by these methods can be any target gene(s) of interest. In some examples, where a single target gene is disrupted, the target gene can be the TCR alpha constant region gene. In some examples where two target genes are disrupted, the target genes can be the TCR alpha constant region gene and the beta-2 microglobulin gene.

In certain embodiments, the presently disclosed methods further comprise introducing into the immune cells a template nucleic acid comprising an exogenous polynucleotide. The cleavage site generated by the engineered nuclease can allow for homologous recombination of the exogenous polynucleotide directly into the target gene. In some embodiments, the recognition sequence is in a target gene, such as those described previously above, and expression of a polypeptide encoded by the target gene is disrupted by insertion of the exogenous polynucleotide. For example, in particular embodiments, the target gene is a TCR alpha constant region gene, and insertion of an exogenous polynucleotide into a cleavage site in the TCR alpha constant region gene results in expression of a polypeptide encoded by the polynucleotide (e.g., a CAR or exogenous TCR), and disrupts expression of the TCR alpha subunit, which subsequently prevents assembly of the endogenous TCR on the cell surface.

In other embodiments, the recognition sequence for insertion of the exogenous polynucleotide is within a safe harbor locus. As used herein, the phrase “safe harbor locus” refers to chromosomal loci where exogenous nucleic acid inserts can be stably and reliably expressed in all tissues of interest without overtly altering cell behavior or phenotype (i.e., without any deleterious effects on the host cell).

In some embodiments, the exogenous polynucleotide comprises a 5′ homology arm and a 3′ homology arm flanking the elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

In various embodiments, the exogenous polynucleotide can comprise a coding sequence for a polypeptide of interest. It is envisioned that the coding sequence can be for any polypeptide of interest. In particular embodiments of the method, the polypeptide of interest can be a chimeric antigen receptor or an exogenous T cell receptor. In still other embodiments, the exogenous polynucleotide can encode the wild-type or modified version of an endogenous gene of interest.

The template polynucleotide or exogenous polynucleotide described herein can further comprise additional control sequences. For example, the exogenous polynucleotide can include homologous recombination enhancer sequences, Kozak sequences, polyadenylation sequences, transcriptional termination sequences, selectable marker sequences (e.g., antibiotic resistance genes), origins of replication, and the like. Exogenous polynucleotides described herein can also include at least one nuclear localization signal. Examples of nuclear localization signals are known in the art (see, e.g., Lange et al., J. Biol. Chem., 2007, 282:5101-5105).

The template nucleic acid can be introduced into the immune cells via any method known in the art for delivery of nucleic acids into cells. For embodiments in which the template polynucleotide is delivered in DNA form and encodes a polypeptide of interest, it can be operably linked to a promoter to facilitate transcription of the polypeptide of interest. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). The exogenous coding sequence can also be operably linked to a synthetic promoter. Synthetic promoters can include, without limitation, the JeT promoter (WO 2002/012514).

In another particular embodiment, the template polynucleotide can comprise a single-stranded DNA template. The single-stranded DNA can further comprise a 5′ and/or a 3′ AAV inverted terminal repeat (ITR) upstream and/or downstream of the sequence encoding the polypeptide of interest. In other embodiments, the single-stranded DNA can further comprise a 5′ and/or a 3′ homology arm upstream and/or downstream of the sequence encoding the polypeptide of interest.

In yet another particular embodiment, the template polynucleotide comprises a linearized DNA template. In some examples, a plasmid DNA encoding a polypeptide of interest can be digested by one or more restriction enzymes such that the circular plasmid DNA is linearized prior to being introduced into a cell.

In some embodiments, the template nucleic acid is introduced into the immune cells using a recombinant DNA construct. In some embodiments, the recombinant DNA construct is encapsulated in a lipid nanoparticle and in some of these embodiments, the recombinant DNA construct is encapsulated in a lipid nanoparticle that further comprises the mRNA encoding the engineered nuclease.

In certain embodiments, the template nucleic acid is introduced into the immune cells (e.g., T cells) using a viral vector (i.e., a recombinant virus). Such vectors are known in the art and include recombinant retroviruses, recombinant lentiviruses, recombinant adenoviruses, and recombinant adeno-associated viruses (AAVs) (reviewed in Vannucci, et al. (2013 New Microbiol. 36:1-22). Recombinant AAVs useful in the invention can have any serotype that allows for transduction of the virus into the cell. In particular embodiments, recombinant AAVs have a serotype of AAV2 or AAV6. Recombinant AAVs can also be self-complementary such that they do not require second-strand DNA synthesis in the host cell (McCarty, et al. (2001) Gene Ther. 8:1248-54). In particular embodiments, the viral vector (i.e., recombinant virus) comprising the template polynucleotide is a recombinant AAV.

The template nucleic acid can be introduced into the immune cells prior to contacting the immune cells with the lipid nanoparticles, after contacting the cells with the lipid nanoparticles, or simultaneously with contacting the cells with the lipid nanoparticles. In certain examples, the template nucleic acid can be introduced into the immune cells between 0 and about 48 hours, 0 to about 24 hours, or about 24 to about 48 hours, after contacting the cells with the lipid nanoparticles. In a particular example, the template nucleic acid can be introduced into the immune cells between 24 and 48 hours after contacting the cells with the lipid nanoparticles.

Immune cells (e.g., T cells) modified by the present invention may require activation prior to contacting the cells with the lipid nanoparticles and/or introduction of the target polynucleotide. For example, T cells can be contacted with anti-CD3 and anti-CD28 antibodies that are soluble or conjugated to a support (i.e., beads) for a period of time sufficient to activate the cells.

Genetically-modified immune cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

Previously known standard methods of contacting immune cells with mRNA encoding an engineered nuclease utilized electroporation to enhance cellular permeability and allow penetration of the mRNA into the immune cell. The process of electroporation requires that immune cells be removed from their vessel, centrifuged, re-suspended in specific buffers, and moved to new vessels. The introduction of the template nucleic acid can require further isolation and movement of cells if different media conditions are required. By comparison, the methods disclosed herein allow for a greatly simplified process by which nuclease mRNA can be introduced into immune cells in combination with a template nucleic acid (e.g., one encoding a polypeptide of interest, such as CAR or exogenous TCR). In some embodiments, the immune cells are not transferred to a new vessel between the step of contacting the cells with the lipid nanoparticles and the introduction of the template nucleic acid. In some embodiments, the immune cells are not centrifuged between the step of contacting the immune cells with the lipid nanoparticles and the step of introducing the template nucleic acid. For example, in particular embodiments, the immune cells can be contacted with lipid nanoparticles and an AAV comprising the template nucleic acid in the same vessel, avoiding the need for centrifugation, re-suspension, and movement between multiple vessels.

### 2.5 Nuclease mRNA

The mRNA encoding an engineered nuclease can be produced using methods known in the art such as in vitro transcription. In some embodiments, the mRNA comprises a 5′ cap. Such 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, Calif.), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element.

The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

### 2.6 Chimeric Antigen Receptors and Exogenous T Cell Receptors

In certain embodiments, the exogenous polynucleotide inserted into a nuclease cleavage site encodes a chimeric antigen receptor (CAR). Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

In some embodiments, a CAR useful in the invention comprises an extracellular ligand-binding domain having specificity for a cancer cell antigen (i.e., an antigen expressed on the surface of a cancer cell). The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR can include those associated cancer cells. In some embodiments, a CAR is engineered to target a cancer-specific antigen of interest by way of engineering a desired ligand-binding moiety that specifically binds to an antigen on a cancer cell. In the context of the present disclosure, “cancer antigen” or “cancer-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79b, CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), IL1RAP, CS1, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human. In certain embodiments, the scFv comprises a heavy chain variable (VH) domain and a light chain variable (VL) domain from a monoclonal antibody having specificity for a tumor cell antigen.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

A CAR can comprise a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRIIIa receptor or IgG1. In certain examples, the hinge region can be a CD8 alpha domain.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

The CAR can be specific for any type of cancer cell. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs that target, for example, CD19, CD20, CD22, and/or ROR1. In some non-limiting examples, a genetically-modified immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, and multiple myeloma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In other embodiments, the exogenous polynucleotide that is introduced into the immune cells can encode an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest. For example, exogenous TCRs can have specificity for any cancer antigen or any type of cancer cell described herein.

### 2.7 Cell Populations

In one aspect of the present invention, populations of genetically-modified immune cells are provided that are prepared according to the methods disclosed herein. Surprisingly, according to the present disclosure, several characteristics of genetically-modified immune cell populations, prepared using the presently disclosed methods, are unexpectedly improved. For example, populations of genetically-modified T cells prepared according to the disclosed methods exhibit a number of improved properties when compared to T cell populations produced using electroporation for the delivery of nuclease mRNA. These include, for example, the production of T cell populations with advantageous ratios of CD4+ T cells to CD8+ T cells, an improvement in the number of CD4+ cells that maintain a central memory phenotype, a reduction in the number of CD4+ cells that exhibit an effector phenotype, and an overall increase in the number of gene-edited T cells when compared to populations made using electroporation.

Typically, during the production of a clinical product, genetically-modified immune cells, such as T cells, remain in culture for expansion for up to 3 weeks. During this time, it has been observed that the phenotype of the cell population changes in multiple ways. For example, following electroporation, the ratio of CD4+ T cells to CD8+ T cells shifts toward a primarily CD8+ population, sometimes exhibiting a ratio as low as 0.2 (CD4+/CD8+). Surprisingly, when cultured for one to two weeks after contacting the T cells with mRNA-containing lipid nanoparticles, populations of genetically-modified T cells (which are “electroporation naïve”) exhibit a CD4+ to CD8+ ratio that remains closer to a 1.0 ratio, or even skews in favor of CD4+ cells at ratios above 1.0. For example, ratios of CD4+ to CD8+ T cells can range between about 0.8 and about 1.6, or about 0.8, about 0.9, about 1.0, about 1.1, about 1.2, about 1.3, about 1.4, about 1.5, or about 1.6, or higher. Such ratios of CD4+ T cells to CD8+ T cells can be observed between 7 to 14 days in culture after the T cells have been contacted with the mRNA-containing lipid nanoparticles, or about 7 days, about 8 days, about 9 days, about 10 days, about 11 days, about 12 days, about 13 days, or about 14 days.

Further, rested T cells generally exhibit a naive or central memory phenotype that is advantageous for a clinical CAR T product. As T cells become more activated, either in culture or via antigen exposure, they transition to an effector phenotype, which is less advantageous. Surprisingly, by comparison to the use of electroporation, the methods disclosed herein produce a population of genetically-modified T cells wherein higher percentages of CD4+ T cells in the population exhibit a central memory phenotype when cultured for one to two weeks after being contacted with lipid nanoparticles comprising nuclease mRNA. As used herein, the phrase “central memory phenotype T cells” refers to T cells that express CD45RO, CCR7, and CD62L. In some embodiments, at least about 50%, at least about 60%, at least about 70%, at least about 80%, or at least about 90% of the genetically-modified CD4+ T cells in the population prepared using the presently disclosed methods exhibit a central memory phenotype after about 7 days, about 8 days, about 9 days, about 10 days, about 11 days, about 12 days, about 13 days, about 14 days, or more in culture after being contacted with lipid nanoparticles comprising nuclease mRNA. In particular embodiments, between about 65% and about 90% of CD4+ T cells in the population exhibit a central memory phenotype. In some embodiments, between about 65% and 84% of CD4+ T cells in the population exhibit a central memory phenotype. In certain embodiments, about 65%, about 66%, about 67%, about 68%, about 69%, about 70%, about 71%, about 72%, about 73%, about 74%, about 75%, about 76%, about 77%, about 78%, about 79%, about 80%, about 81%, about 82% about 83%, about 84%, about 85%, about 86%, about 87%, about 88%, about 89%, or up to about 90% of T cells in the population exhibit a central memory phenotype. In some embodiments, the genetically-modified immune cells are genetically-modified T cells expressing a chimeric antigen receptor or exogenous T cell receptor, wherein the genetically-modified T cells do not have detectable cell-surface expression of an endogenous T cell receptor due to the disruption of a TCR alpha gene, a TCR alpha constant region gene, a TCR beta gene, and/or a TCR beta constant region gene.

In some embodiments, between about 3% and about 10% of the genetically-modified CD4+ T cells in the population prepared using the presently disclosed methods exhibit an effector phenotype after about 7 days, about 8 days, about 9 days, about 10 days, about 11 days, about 12 days, about 13 days, about 14 days, or more in culture after being contacted with lipid nanoparticles comprising nuclease mRNA. In particular embodiments, about 3%, about 4%, about 5%, about 6%, about 7%, about 8%, about 9%, or about 10% of CD4+ T cells in the population exhibit an effector phenotype.

In yet another aspect, methods disclosed herein can produce a population of genetically-modified immune cells that are electroporation naïve, wherein the genetically-modified immune cells comprise a target gene modified by an engineered nuclease to disrupt expression of an endogenous polypeptide encoded by the target gene.

In various embodiments, the methods disclosed herein can produce populations of genetically-modified immune cells (e.g., T cells) wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified cell described herein, such as a genetically-modified T cell.

In certain embodiments, the genetically-modified immune cells (e.g., T cells) produced according to the methods disclosed herein express a CAR or an exogenous TCR, and do not have detectable cell-surface expression of an endogenous TCR, such as an alpha/beta TCR (i.e., are TCR-) due to the disruption of a TCR alpha gene, a TCR alpha constant region gene, a TCR beta gene, and/or a TCR beta constant region gene. In particular examples, populations can be prepared according the present methods wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are both TCR- and CAR+.

In some examples, the invention provides a population of immune cells, wherein between about 5% and about 80%, between about 10% and about 80%, between about 20% and about 80%, between about 30% and about 80%, between about 40% and about 80%, between about 50% and about 80%, between about 55% and about 80%, between about 60% and about 80%, between about 65% and about 80%, between about 70% and about 80%, or between about 75% and about 80% of the immune cells in the population are genetically-modified immune cells (e.g., T cells) prepared by the methods described herein, wherein the genetically-modified immune cells comprise a disrupted TCR alpha gene, a disrupted TCR alpha constant region gene, a disrupted TCR beta gene, or a disrupted TCR beta constant region gene. In some examples, about 5%, about 10%, about 20%, about 30%, about 40%, about 50%, about 55%, about 60%, about 65%, about 70%, about 75%, or about 80% of the immune cells in the population are genetically-modified immune cells (e.g., T cells) prepared by the methods described herein, wherein the genetically-modified immune cells comprise a disrupted TCR alpha gene, a disrupted TCR alpha constant region gene, a disrupted TCR beta gene, or a disrupted TCR beta constant region gene.

In certain examples, the invention provides a population of immune cells, wherein between about 5% and about 65%, between about 10% and about 65%, between about 20% and about 65%, between about 30% and about 65%, between about 40% and about 65%, between about 45% and about 65%, between about 50% and about 65%, between about 55% and about 65%, or between about 60% and about 65%, of the immune cells in the population are genetically-modified immune cells (e.g., T cells) prepared by the methods described herein, wherein the genetically-modified immune cells comprise a disrupted TCR alpha gene, a disrupted TCR alpha constant region gene, a disrupted TCR beta gene, or a disrupted TCR beta constant region gene, and express a chimeric antigen receptor or an exogenous TCR. In some examples, about 5%, about 10%, about 20%, about 30%, about 40%, about 45%, about 50%, about 55%, about 60%, or about 65% of the immune cells in the population are genetically-modified immune cells (e.g., T cells) prepared by the methods described herein, wherein the genetically-modified immune cells comprise a disrupted TCR alpha gene, a disrupted TCR alpha constant region gene, a disrupted TCR beta gene, or a disrupted TCR beta constant region gene, and express a chimeric antigen receptor or an exogenous TCR.

### 2.8 Pharmaceutical Compositions

The invention also provides pharmaceutical compositions comprising a pharmaceutically-acceptable carrier and a genetically-modified immune cell of the invention, or a population of genetically-modified immune cells, wherein the population of genetically-modified immune cells is prepared according to the method disclosed herein. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which promote in vivo cell proliferation and engraftment of genetically-modified immune cells (e.g., T cells). Pharmaceutical compositions comprising genetically-modified immune cells of the invention can be administered in the same composition as an additional agent or biological molecule or, alternatively, can be co-administered in separate compositions.

The present disclosure also provides genetically-modified immune cells, or populations thereof, described herein for use as a medicament. The present disclosure further provides the use of genetically-modified immune cells or populations thereof described herein in the manufacture of a medicament for treating a disease in a subject in need thereof. In one such aspect, the medicament is useful for cancer immunotherapy in subjects in need thereof.

Pharmaceutical compositions of the invention can be useful for treating any disease state that can be targeted by adoptive immunotherapy. In a particular embodiment, the pharmaceutical compositions and medicaments of the invention are useful in the treatment of cancer including, for example, types of cancer described elsewhere herein.

In some of these embodiments wherein cancer is treated with the presently disclosed genetically-modified cells or populations thereof, the subject administered the genetically-modified cells, or populations thereof, is further administered an additional therapeutic, such as radiation, surgery, or a chemotherapeutic agent.

### 2.9 Kits

Another aspect of the invention is a kit for transfecting a eukaryotic cell with mRNA. In some embodiments, the kit includes an apolipoprotein and any lipid nanoparticle composition described herein. Exemplary and non-limiting apoliproteins include is apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)) protein. In some embodiments, the apolipoprotein is ApoE. In certain embodiments, the ApoE is ApoE2, ApoE3, or ApoE4. In particular embodiments, the ApoE is ApoE2. In other embodiments, the ApoE is ApoE3. In certain embodiments, the ApoE is ApoE4. In some embodiments, the apolipoprotein and the lipid nanoparticle composition are provided together in a vial or are provided in one or more separate vials. In some further embodiments, the kit includes packaging and instructions for use thereof.

### 2.10 Methods of Administering Genetically-Modified Immune Cells

Another aspect provided herein are methods of treatment comprising administering an effective amount of the genetically-modified immune cells, or populations thereof, of the present disclosure to a subject in need thereof. In particular embodiments, the pharmaceutical compositions described herein are administered to a subject in need thereof. For example, an effective amount of a population of cells can be administered to a subject having a disease. In particular embodiments, the disease can be cancer, and administration of the genetically-modified immune cells of the invention represent an immunotherapy. The administered cells are able to reduce the proliferation, reduce the number, or kill target cells in the recipient. Unlike antibody therapies, genetically-modified immune cells of the present disclosure are able to replicate and expand in vivo, resulting in long-term persistence that can lead to sustained control of a disease.

In particular embodiments of the presently disclosed methods, the subject can be a mammal, such as a human.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, the agent is infused over a period of less than about 12 hours, 6 hours, 4 hours, 3 hours, 2 hours, or 1 hour. In still other embodiments, the infusion occurs slowly at first and then is increased over time.

In some embodiments, a genetically-modified immune cell or population thereof of the present disclosure targets a tumor (i.e., cancer) antigen for the purposes of treating cancer including, for example, types of cancer described elsewhere herein.

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified cells or populations thereof described herein is administered at a dosage of 10⁴ to 10⁹ cells/kg body weight, including all integer values within those ranges. In further embodiments, the dosage is 10⁵ to 10⁷ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, administration of genetically-modified immune cells or populations thereof of the present disclosure reduce at least one symptom of a target disease or condition. For example, administration of genetically-modified T cells or populations thereof of the present disclosure can reduce at least one symptom of a cancer. Symptoms of cancers are well known in the art and can be determined by known techniques.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Lipid Nanoparticle (LNP) Formulations for Delivery of eGFP mRNA into T Cells

### 1. Lipid Nanoparticle Formulations

The lipid materials used for the formulation of lipid nanoparticles in this experiment comprised one of two formulations containing A) DODMA, Cholesterol, DSPC, and DMG-PEG dissolved at a 50:38.5:10:1.5 molar ratio in ethanol; or B) SS-33/3APO5 (NOF), Cholesterol, DSPC, and DMG-PEG dissolved at a 50:40:8:2.5 molar ratio in ethanol at a total lipid concentration of 30 mM (high N:P=8) or 15 mM (low N:P=4) depending on desired N to P ratio. The mix of lipids was stored at −80° C. and thawed by heating to 50° C. in a heat block. The lipid mix was removed from the heat source and vortexed immediately before use in formulation.

The mRNA material coding for the eGFP in this experiment consisted of a clean cap 1 structure with no uridine substitution, obtained commercially from TriLink Cat #L-7601. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via a Precision Nanosystems SPARK mixer was performed. The final solution in the exchange buffer reservoir was collected and analyzed for physical characteristics such as size, PDI, and zeta potential, as well as for encapsulation efficiency. Formulations (2 μg/mL dose) were added to human donor T cells (with or without ApoE at 1 μg/mL) to asses efficacy of LNP formulations to deliver mRNA encoding eGFP and produce functional eGFP protein in human donor T cells, measured by GFP fluorescence via flow cytometry analysis.

### 2. T Cell Culture and Transfection

An apheresis sample was obtained from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28; Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 μg of mRNA encoding eGFP and consisting of a clean cap 1 structure with no uridine substitution (TriLink). Other samples of 0.5e6 cells were treated with 1 μg/mL of ApoE, or no ApoE, then transfected with 2 μg/mL of Low N:P LNP or High N:P LNP formulation as disclosed herein.

### 3. Analysis

Flow cytometry was used to assess live cell count, total cell count of live eGFP+ cells, % of live cells that are eGFP+, and GFP MFI in eGFP+ cells. At 24 and 72 hours, an aliquot of cells was collected, stained with Ghost Dye Violet 510 (Tonbo Biosciences), washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live. GFP expression was obtained from live cell population.

### 4. Results

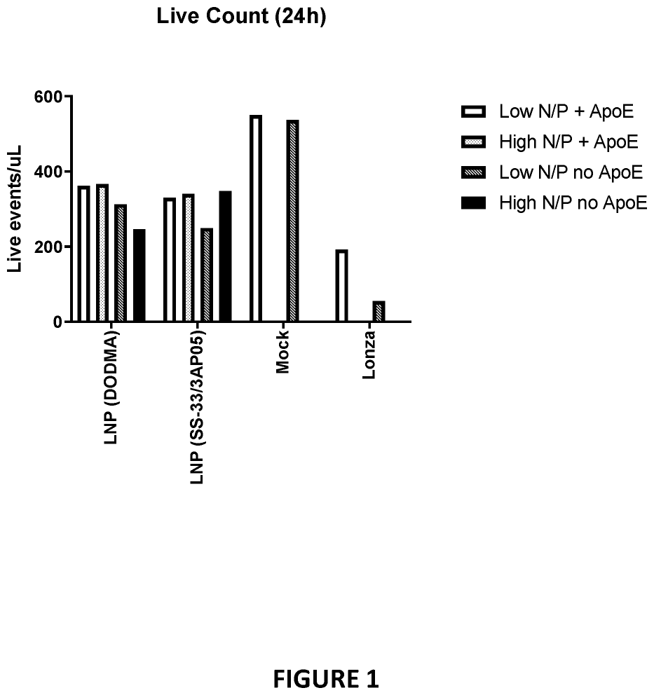

The results of the experiments in this example are summarized in FIGS. 1 to 5, which demonstrated that LNP formulations can achieve transfection of human donor T cells. As illustrated in FIG. 1, at 24 h post transfection, total live cell counts were significantly higher in the LNP transfection groups (300-380 live cells/μL) compared to the Lonza electroporation group (50-200 live cells/μL), in which the electroporation starting cell density of 1e6 cells/mL was higher (2×) compared to LNP transfection groups.

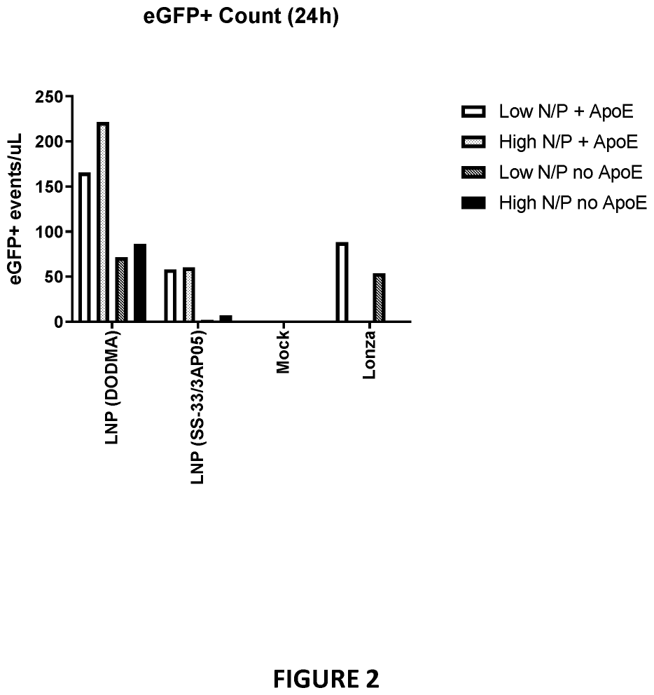

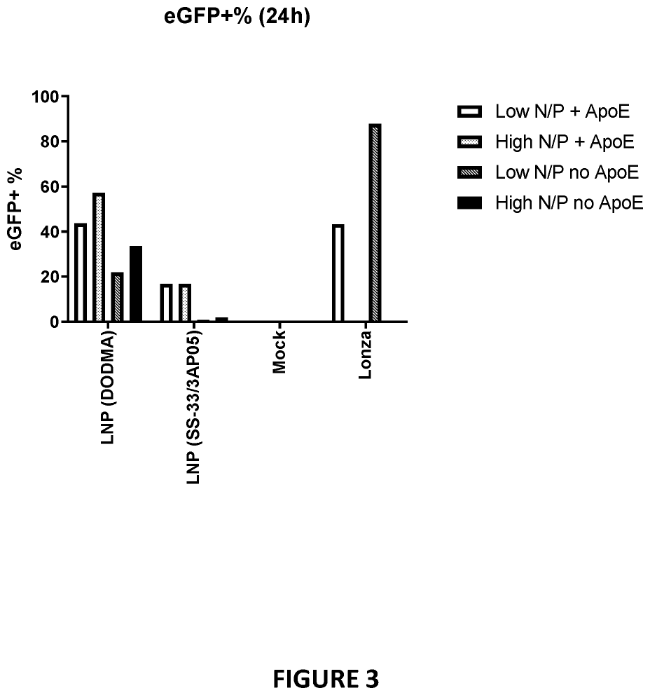

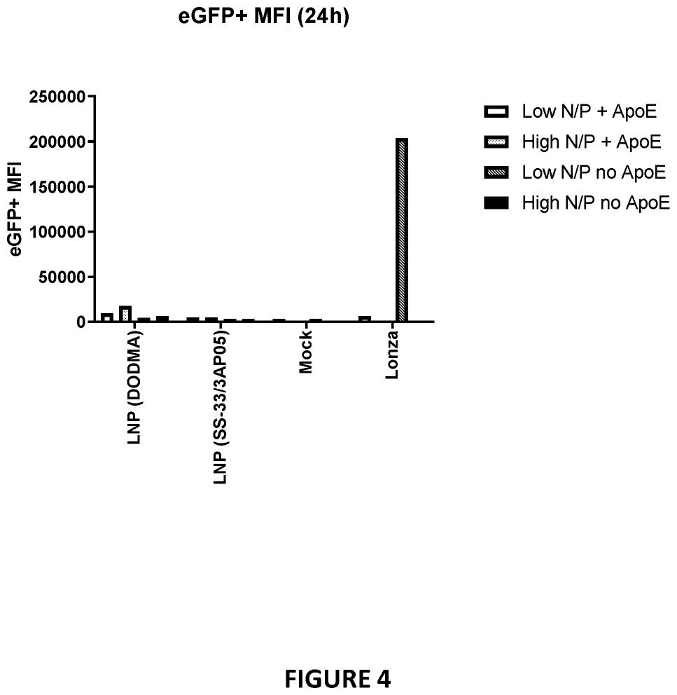

The total eGFP+ cell count was found to be significantly higher (>3×) for the LNP groups with ApoE in the cell culture media, and a slight increase in total GFP+ cells was observed for the high N:P groups compared to low N:P (FIG. 2). Furthermore, the LNP-containing DODMA showed 3× more total eGFP+ cells compared to electroporation. The electroporation control demonstrated (FIG. 3) the highest percentage of live cells that were eGFP+(>80%) compared to the DODMA LNP (55%) and NOF LNP (35%). Further, according to FIG. 4, the measured MFI of these eGFP+ cells showed a significantly higher level in electroporation (>200K) compared to LNP (DODMA, <10K) and LNP (SS-33/APO5, <1K).

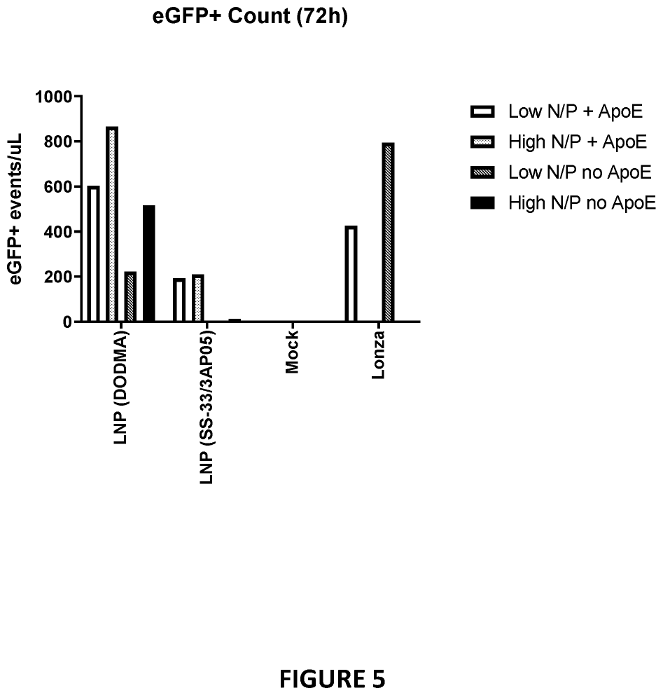

At 72 h post transfection, the total eGFP+ cell count was found to be comparable between the DODMA LNP at high N:P=8 (850 eGFP+ cells/μL) and the electroporation control (800 eGFP+ cells/μL), while the NOF LNP formulation resulted in a lower total number of eGFP+ cells (200 eGFP+ cells/μL at N:P=8) and below the limit of quantitation (LOQ) for N:P=4 (FIG. 5). Notably, the addition of ApoE produced a 50-200% increase in total eGFP+ cells in the DODMA LNP group but showed negative effects in the electroporation group.

It was observed at 24 hr post-transfection that the MFI of the electroporated eGFP+ cells was substantially higher than the MFI of the LNP transfected cells, indicating that the level of eGFP expression in each eGFP+ cell was still superior using electroporation. However, at 24 h post-transfection, the DODMA and NOF LNP formulations were both capable of producing a total number of eGFP+ cells that was equal to, or greater than, the total number achieved using electroporation, even though the electroporated group began with twice as many cells. Additionally, at 72 h, transfection with the DODMA LNP still exhibited a comparable total number of eGFP+ cells compared to electroporation, particularly cells produced using the DODMA LNP at a N:P of 8 in the presence of ApoE. Surprisingly, this example also demonstrated that the addition of ApoE produced a clear improvement in LNP transfection of T cells. With the addition of ApoE, transfection with the NOF LNP (SS-33/3APO5) produced a readily detectable number of eGFP+ cells, whereas no eGFP was detected in the absence of ApoE. Similarly, the addition of ApoE improved the transfection efficiency of the DODMA LNP by 2-3 fold compared to groups without ApoE.

### Example 2

### LNP Formulations for Delivery of Nuclease mRNA into T Cells

### 1. Lipid Nanoparticle Formulations

The lipid materials used for the formulation of lipid nanoparticles in this experiment comprised DODMA, Cholesterol, DSPC, and DMG-PEG dissolved at a 50:38.5:10:1.5 molar ratio in ethanol at a total lipid concentration of 30 mM (high N:P=8) or 15 mM (low N:P=4) depending on desired N to P ratio. The mix of lipids was stored at −80° C. and thawed by heating to 50° C. in a heat block. The lipid mix was removed from the heat source and vortexed immediately before use in formulation.

In this example, the encoded nuclease was an engineered meganuclease referred to as TRC 1-2L.1592, which comprises SEQ ID NO: 2 and has a recognition sequence of SEQ ID NO: 3 within the T cell receptor (TCR) alpha constant region (TRAC) gene. Cleavage of its recognition sequence in TRAC has previously been shown to knock out expression of the TCR alpha subunit, preventing assembly the endogenous TCR complex on the cell surface. The mRNA material coding for the ARCUS TRC nuclease comprised an ARCA cap structure with no uridine substitution. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency. Formulations (0.5 and 2 μg/mL dose) were added to human donor T cells to assess efficacy of LNP formulations to deliver mRNA encoding a TRC nuclease to edit at the TRAC locus and reduce TCR on the cell surface, measured by CD3 staining and flow cytometry analysis.

### 2. T Cell Culture and Transfection

An apheresis sample was obtained from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28-Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 0.5 or 2 μg of mRNA encoding ARCUS TRC nuclease and consisting of an ARCA cap structure with no uridine substitution (TriLink). Other samples of 0.5e6 cells/mL were treated with 1 μg/mL of ApoE then transfected with 0.5 or 2 μg/mL of LNP formulation.

### 3. Analysis

Flow cytometry was used to assess live cell counts and CD3 knock-out (i.e., TCR-negative) cell counts. At 48 hours an aliquot of cells was collected, stained with Ghost Dye Violet 510 (Tonbo Biosciences) and mouse anti-human CD3-BV711, clone UCHT1 (Becton Dickinson), washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live. CD3 knock-out counts were obtained from live cell population.

### 4. Results

Generally, this example determined that LNP formulations can be used to deliver mRNA encoding an engineered nuclease into primary T cells. Further, the study determined the encoded nuclease could be expressed by the cell at levels sufficient for knockout of a target gene.

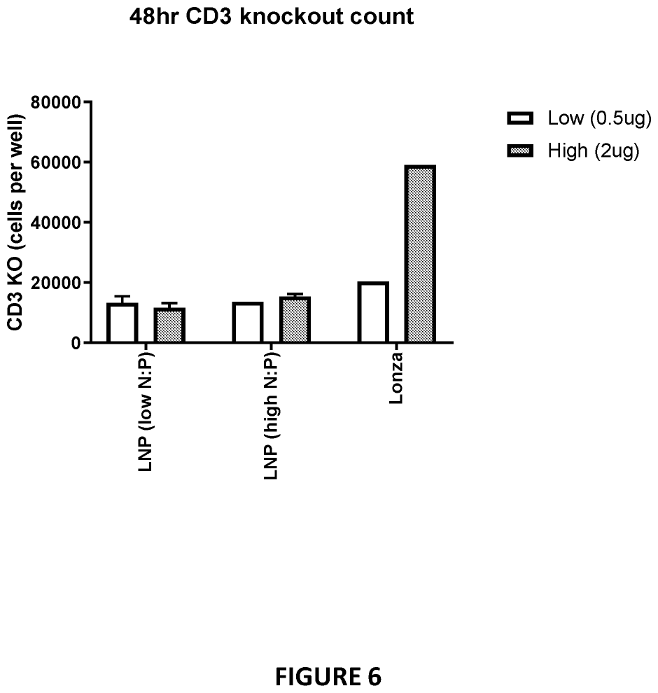

At 48 hours post-transfection of the low dose of mRNA (0.5 μg), CD3 knock out (i.e., TCR knock out) using the DODMA LNP was determined to be approximately 60% (Low N:P) and 55% (High N:P) of the CD3 knockout achieved by using electroporation (FIG. 6). At 48 hours post-transfection of the high dose of mRNA (2 μg), CD3 knockout using the DODMA LNP was approximately 25% (Low N:P) and 30% (High N:P) of the CD3 knockout achieved using electroporation.

The LNP comprising DODMA, Cholesterol, DSPC, and DMG-PEG at a high (8) or low (4) N:P demonstrated potency for transfecting T cells and producing knockout of the TRAC gene. The DODMA LNP was effective at the low dose of mRNA, achieving 60% of the effect observed with electroporation, but apparently requires further optimization to generate more efficient knockout at higher doses of mRNA, where only 30% of the effect produced by electroporation was achieved.

### Example 3

### LNP Formulations for Delivery of eGFP and Nuclease mRNA into T Cells

### 1. Lipid Nanoparticle Formulations

The lipid and buffer materials used for the formulation of Invivofectamine (IVF) were obtained commercially from Thermofisher Cat. #A36155. The mix of lipids and buffer was stored at −20° C. and thawed at room temperature.

The mRNA material coding for the ARCUS TRC nuclease in this experiment consisted of a clean cap 1 structure with substitution of uridine nucleotides with pseudo-uridine, and mRNA material coding for the eGFP consisting of a clean cap 1 structure with no uridine substitution, commercially obtained from Trilink Cat #L-7601. The mRNA was stored at −80° C. and thawed at room temperature. Once thawed, the mRNA was diluted to 2.4 mg/mL in nuclease-free water. The formulation was prepared by mixing 50 μL of complexation buffer with 50 μL of mRNA solution (2.4 mg/mL). This mRNA solution (1.2 mg/mL) was added to 100 μL of lipid mix (Invivofectamine mRNA reagent) in a 1.5 mL Eppendorf tube. The mixture was vortexed immediately to ensure invivofectamine mRNA complexation. The formulation was then incubated for 30 minutes at 50° C. with mild intermittent vortexing. The formulation was then diluted 6-fold by adding 1 mL of sterile RNAse-free PBS (pH=7.4) and mixed well. The final solution was collected and analyzed for physical characteristics such as size, PDI, and zeta potential, as well as for encapsulation efficiency. Formulations (2 μg/mL dose) were added to human donor T cells to assess efficacy of LNP formulations to deliver two different mRNAs, either encoding eGFP or encoding a TRC nuclease, to edit at the TCR locus and reduce TCR on the cell surface, measured by CD3 staining and flow cytometry analysis.

### 2. T Cell Culture and Transfection

An apheresis sample was obtained from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28-Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 μg of mRNA coding for TRC nuclease, consisting of a clean cap 1 structure with substitution of uridine nucleotides with pseudo-uridine, or mRNA coding for eGFP and consisting of a clean cap 1 structure with no uridine substitution (TriLink). Other samples of 0.5e6 cells/mL were treated with 1 μg/mL of ApoE then transfected with 2 μg/mL of LNP IVF formulation with mRNA coding for TRC nuclease or mRNA coding for eGFP.

### 3. Analysis

eGFP expression and CD3 knock-out were assessed by flow cytometry. At 72 hours, an aliquot of cells was collected, stained with Ghost Dye Violet 510 (Tonbo Biosciences) and mouse anti-human CD3-BV711, clone UCHT1 (Becton Dickinson), washed, resuspended in PBS (Gibco) and analyzed on a CytoFLEX LX (Beckman Coulter). Post-run analysis was completed using CytExpert software (Beckman Coulter). Cells were gated on FSC vs SSC scatter followed by singlets then live. CD3 and eGFP expression were obtained from live cell population.

### 4. Results

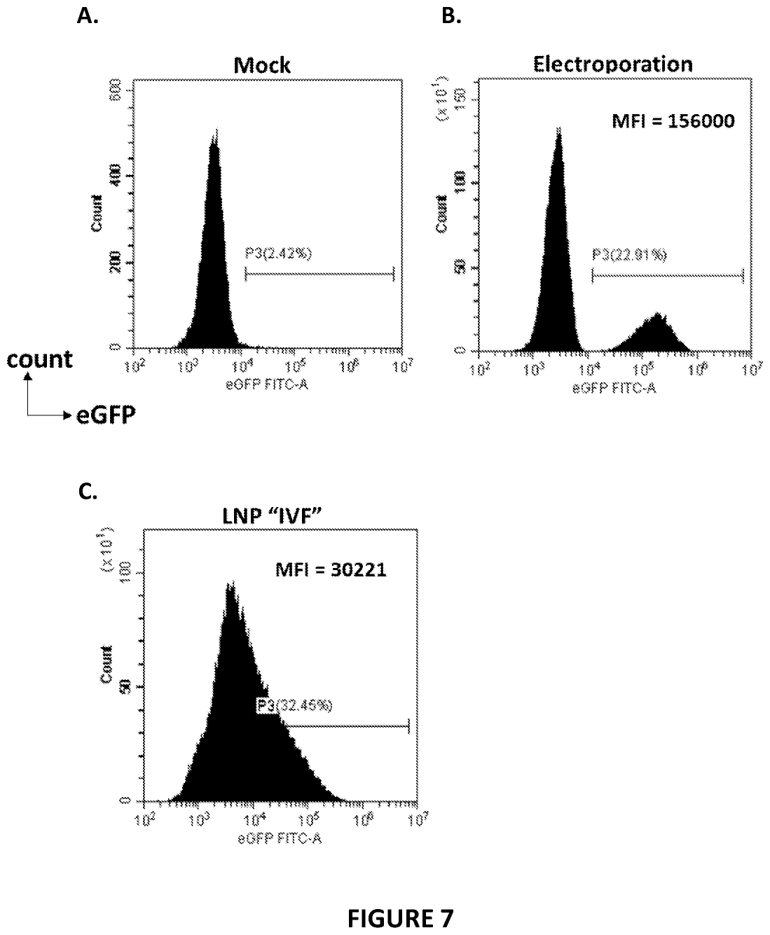

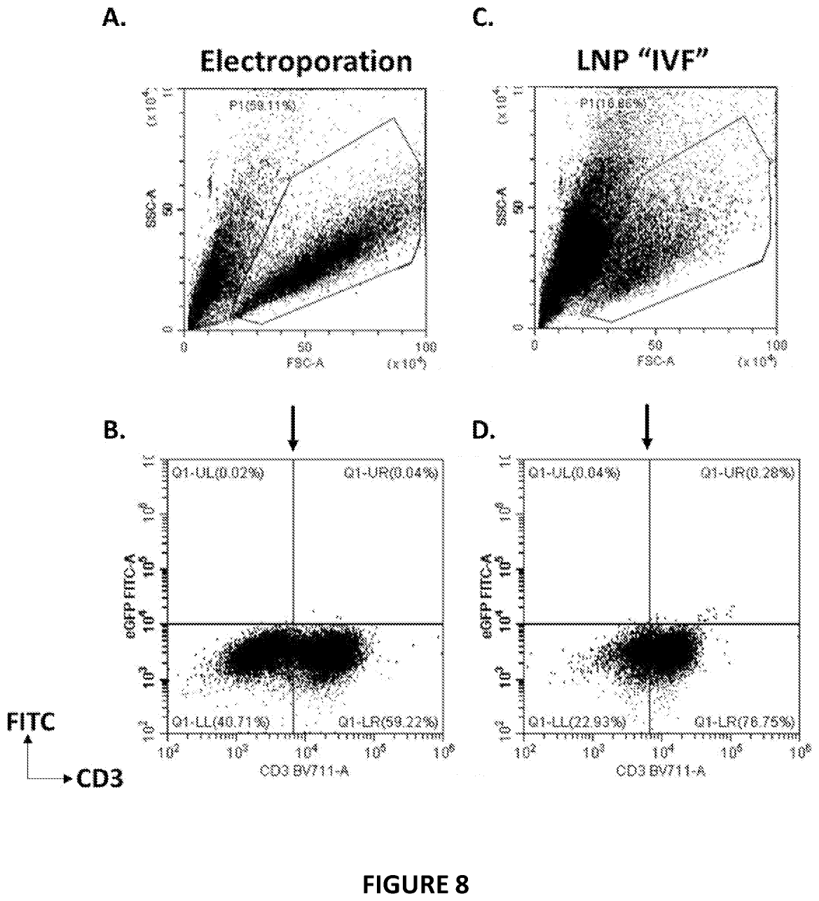

As illustrated in FIG. 7, at 3 days post-transfection of eGFP via IVF LNP or electroporation, the percentage of cells that were found to be eGFP positive were significantly higher in the IVF LNP-transfected group (32%) compared to the electroporation group (23%). It was also observed that the MFI was significantly higher following electroporation (1.56e5) compared to the IVF LNP (3.0e4), similar to the effect observed in Example 1. At 3 days post-transfection of mRNA encoding the TRC meganuclease, it was observed that transfection via the IVF LNP produced a CD3 knockout frequency of 23% (FIG. 8). However, introduction of the nuclease mRNA by electroporation was more efficient, producing a CD3 knockout efficiency of 41%.

The LNP comprising the IVF transfection reagent demonstrated potency for transfecting T cells with mRNA encoding either eGFP (32% eGFP+) or the TRC meganuclease (23% CD3 knockout) into primary T cells.

### Example 4

### LNP Formulations for Delivery of Nuclease mRNA into T Cells

### 1. Lipid Nanoparticle Formulations

The lipid materials used for the formulation of LNP in this experiment consisted of 1) DODMA, Cholesterol, DSPC, and DMG-PEG dissolved at a 50:38.5:10:1.5 molar ratio in ethanol at a total lipid concentration of 30 mM (high N:P=8); or 2) DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80° C. and thawed by heating to 50° C. in heat block. The lipid mix was removed from the heat source and vortexed immediately before use in formulation.

The mRNA material coding for the ARCUS TRC nuclease in this experiment consisted of a clean cap 1 structure with substitution of uridine nucleotides with pseudo-uridine. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 μg of mRNA coding for TRC nuclease, consisting of a clean cap 1 structure with substitution of uridine nucleotides with pseudo-uridine. Other samples of 0.5e6 cells/mL were treated with 1 μg/mL of ApoE then transfected with 2 μg/mL of LNP formulation with mRNA coding for TRC nuclease.

### 3. Analysis

CD3 knock-out was assessed by flow cytometry. On days 3, 7 and 9, an aliquot of cells was collected, stained with Ghost Dye Violet 510 (Tonbo Biosciences) and anti-human CD3-BV421, clone OKT3 (BioLegend), washed, resuspended in PBS (Gibco) and analyzed on a CytoFLEX LX (Beckman Coulter). Post-run analysis was completed using CytExpert software (Beckman Coulter). Cells were gated on FSC vs SSC scatter followed by singlets then live. CD3 expression and MFI were obtained from live cell population.

### 4. Results

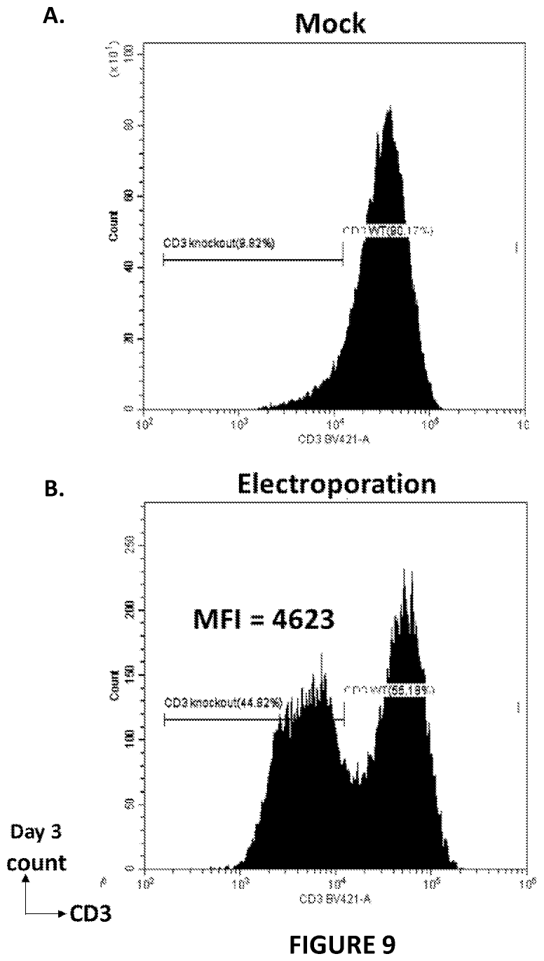

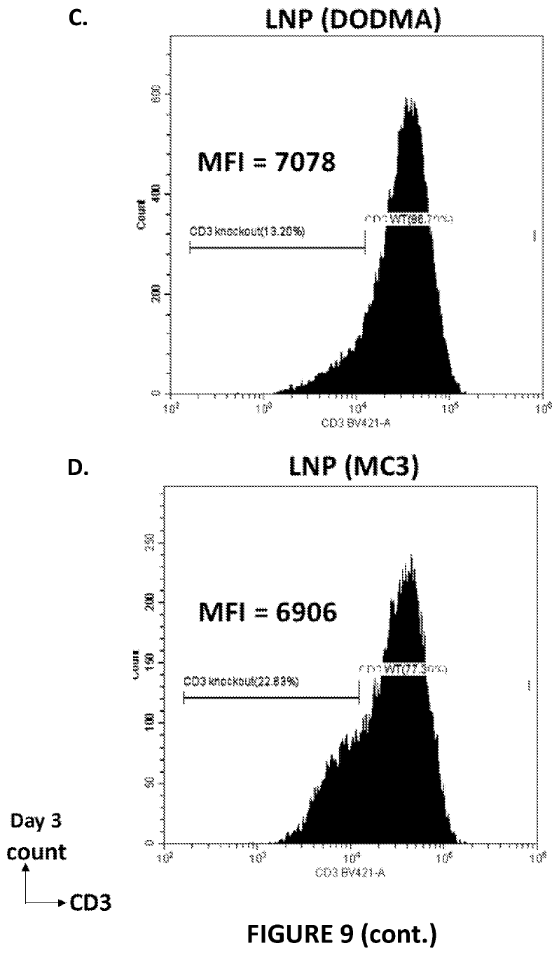

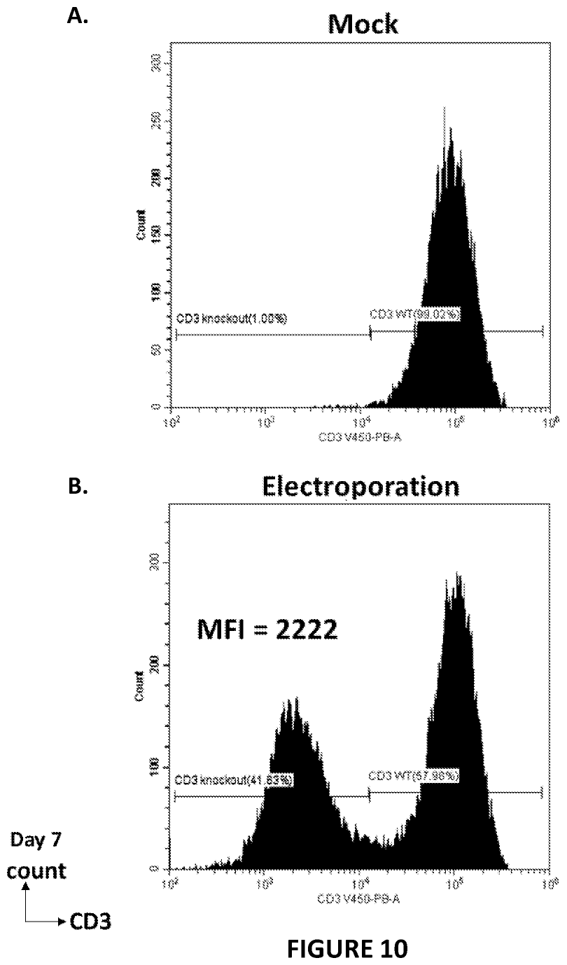

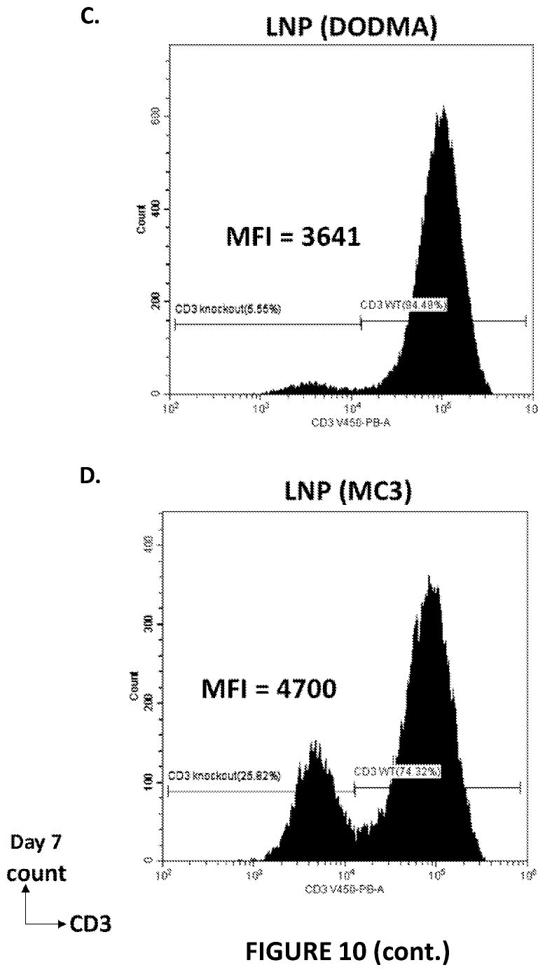

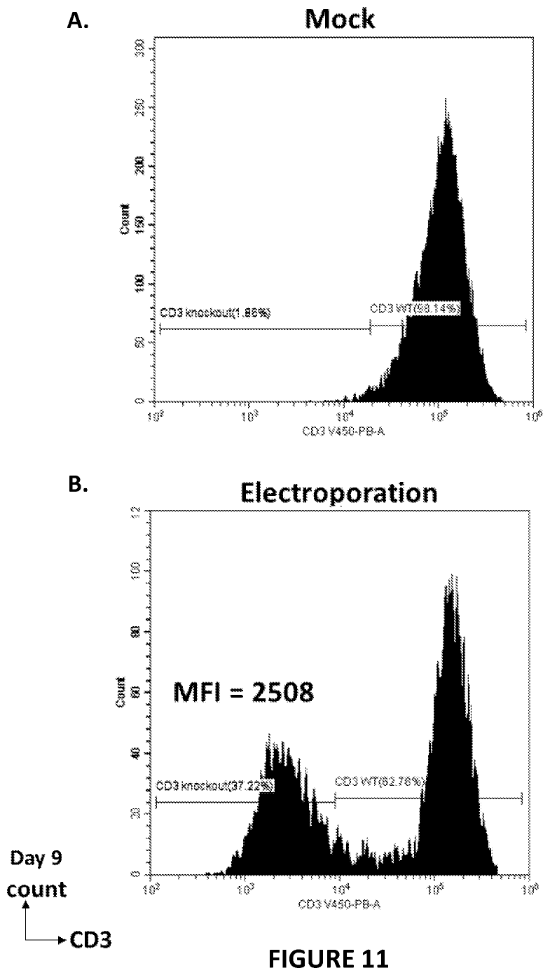

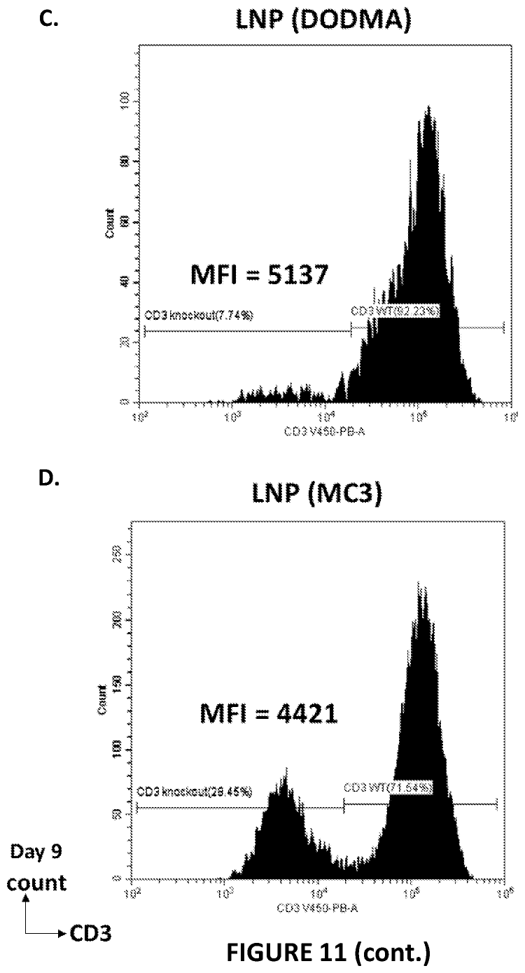

At day 3 of this study, transfection of nuclease mRNA using the MC3 and DODMA LNPs produced CD3 knockout in 23% and 13% of cells, respectively (FIG. 9). By comparison, electroporation was more efficient, generating CD3 knockout in in 44% of cells. At day 7, results showed the level of CD3 knockout cells was maintained post electroporation (42%), while the DODMA LNP showed a reduction in the CD3 knockout population to 6% (FIG. 10). By comparison, cells transfected with the MC3 LNP demonstrated a consistent level of CD3 knockout cells (26%), with a more pronounced reduction in CD3 expression demonstrated by a decrease in MFI to 4700. At day 9, the electroporated group showed a slight decrease in the frequency of CD3 knockout cells to 37% (FIG. 11). The group transfected with the DODMA LNP continued to show a reduced CD3 knockout population (8%), while the MC3 LNP group exhibited a further increased level of CD3 knockout cells (28%), with a more pronounced reduction in CD3 expression demonstrated by a decrease in MFI to 4421.

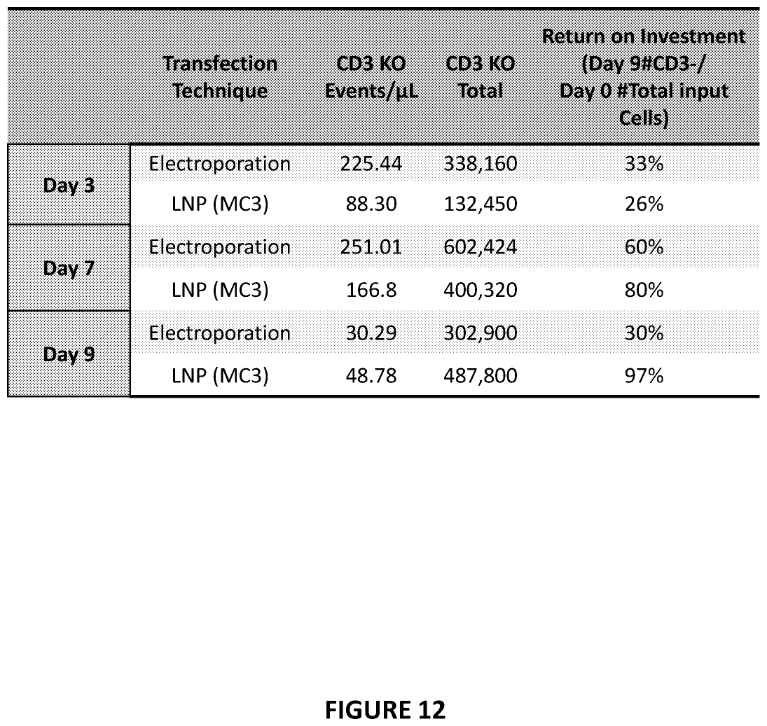

The results of this experiment are summarized in the table of FIG. 12. Use of electroporation produced a faster onset and turnover of CD3 knockout cells versus MC3 LNP as seen by number of total CD3 knockout cells (3.38e5 versus 1.32e5) on day 3. Analysis of day 9 CD3 knockout cell counts demonstrated that the MC3 LNP outperformed electroporation by generating 97% (4.87e5 CD3 knockout cells) of the initial total cells transfected (5e5), compared to electroporation at 30% (3.02e5 CD3 knockout cells) of the initial total cells transfected (1e6).

This experiment demonstrated that a non-viral vector without targeting moieties (MC3 LNP) resulted in efficient mRNA transfection and gene editing in primary T cells at the TCR locus, and reduced TCR on the cell surface, as measured by CD3 staining and flow cytometry analysis. The levels of CD3 knockout were comparable to, or outperformed, those obtained via electroporation, which is the current gold standard for mRNA transfection of T cells. By day 9, transfection with the MC3 LNP had generated more CD3 knockout cells compared to the use of electroporation, even though the MC3 LNP group started with 2-fold fewer T cells at the beginning of the study.

### Example 5

### LNP Formulations for Delivery of Nuclease mRNA for Production of CAR T Cells

### 1. Lipid Nanoparticle Formulations

The lipid materials used for the formulation of LNP in this experiment consisted of DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80° C. and thawed by heating to 50° C. in heat block. The lipid mix was removed from the heat source and vortexed immediately before use in formulation.

The mRNA material coding for the ARCUS TRC nuclease in this experiment consisted of a clean cap 1 structure with substitution of uridine nucleotides with pseudo-uridine. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

In this example, an apheresis sample was obtained from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1 μg of mRNA coding for TRC nuclease, consisting of a clean cap 1 structure with substitution of uridine nucleotides with pseudo-uridine. Other samples of 0.5e6 cells/mL were treated with 1 μg/mL of ApoE then transfected with 2 μg/mL of LNP formulation with mRNA coding for TRC nuclease.

To assess efficacy of the LNP formulations to deliver mRNA encoding a TRC nuclease to edit at the TCR locus and reduce TCR on the cell surface (as measured by CD3 staining and flow cytometry analysis), AAV-7206, carrying an anti-CD19 CAR construct, was added to the culture. Specifically, at different time points post LNP transfection (0-96 hours), AAV containing CAR-T (Anti-CD19) construct was added to cells in culture to determine the optimal time for CAR gene insertion in order to produce a potent CAR T-cell capable of targeted CD19+ cell killing. AAV transduction and CAR template delivery occurred at 0, 24, 48, or 72 hours post LNP transfection.

### 3. Analysis

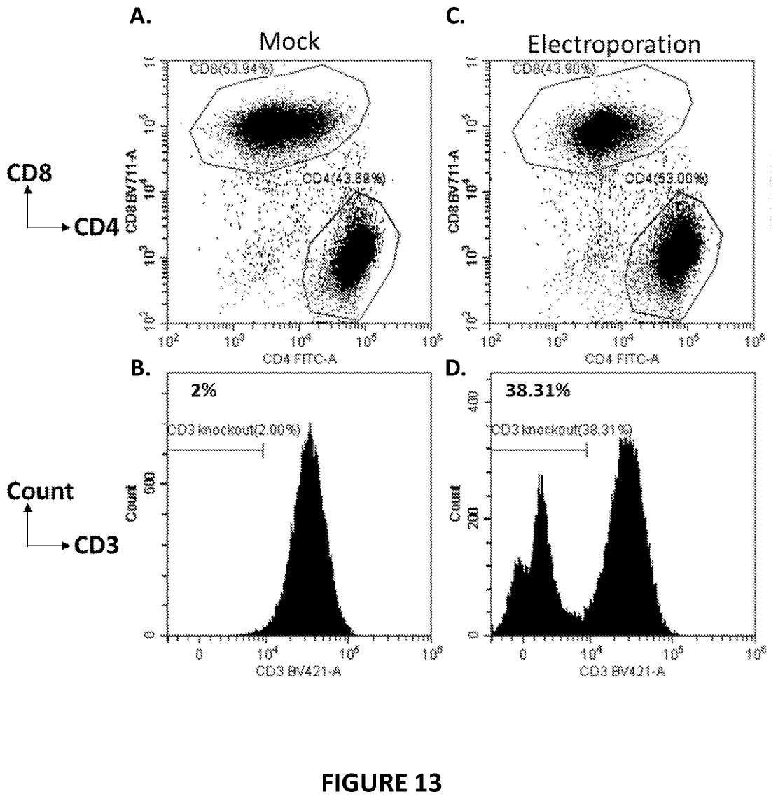

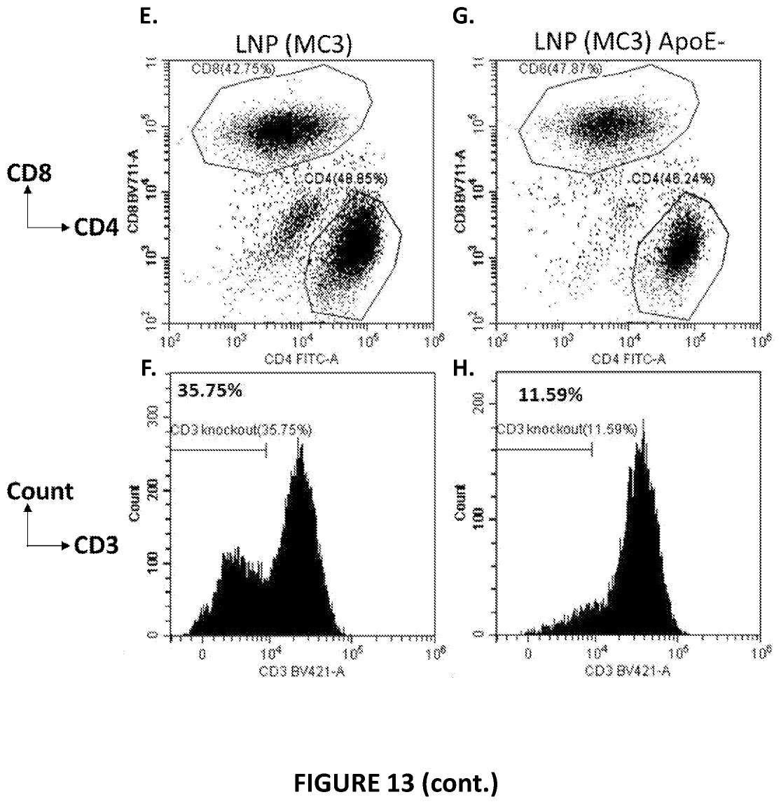

Cell phenotype was assessed by flow cytometry. On days 3, 8 and 10, an aliquot of cells was collected, stained with Ghost Dye Violet 510 (Tonbo Biosciences), anti-human CD3-BV421, clone OKT3 (BioLegend), anti-human CD4-FITC, clone OKT4 (BioLegend), anti-human CD8-BV711, clone RPA-T8 (BioLegend) and anti-FMC63 recombinant antibody-AF647, clone VM16 (BioLegend) washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live. Expression and MFI were obtained from the live cell population. The results of these experiments are summarized in FIGS. 13 to 15.

### 4. Results

Similar levels of CD3 knockout and CD4:CD8 ratios were observed following the use of electroporation or the MC3 LNP 3 days post transfection. Similar to the effect observed in previous examples, transfection of mRNA using the MC3 LNP without ApoE showed a significant reduction in the CD3 knockout population by approximately 66% (35.75% to 11.59%), while the CD4:CD8 ratio remained comparable to the MC3 LNP with ApoE (FIG. 13).

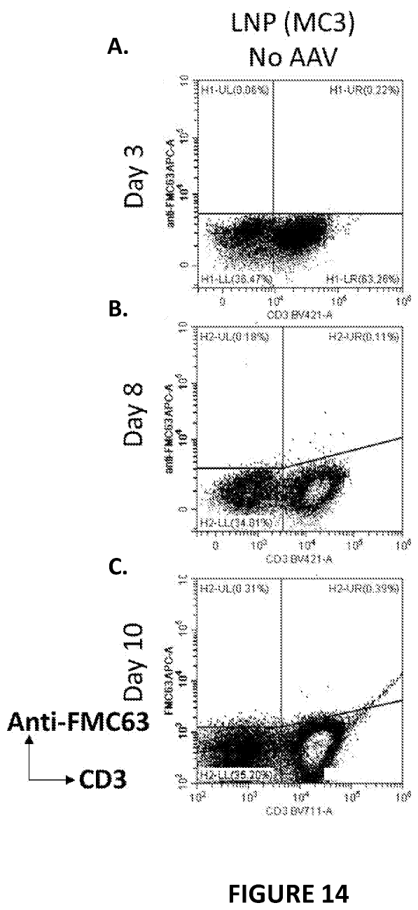

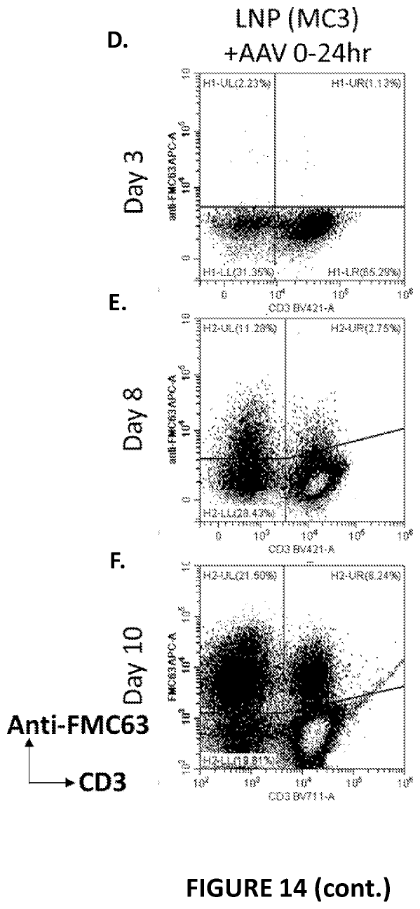

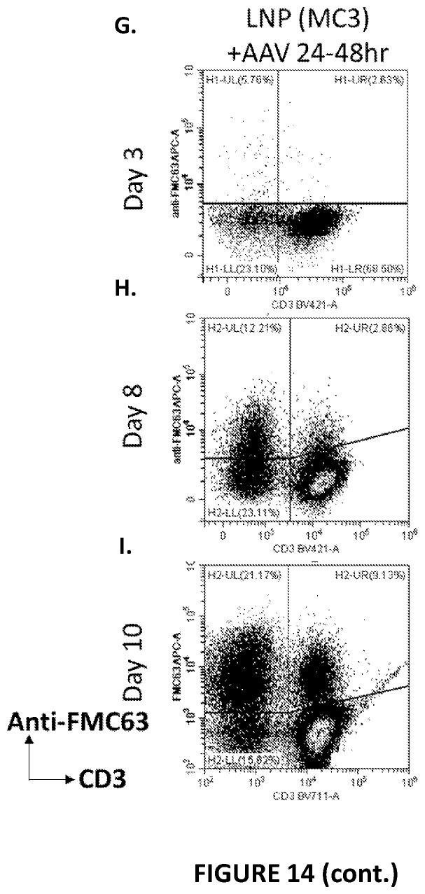

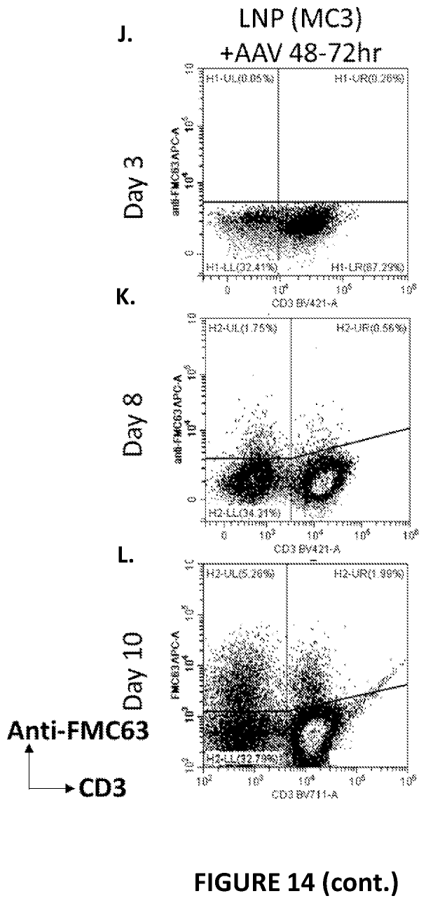

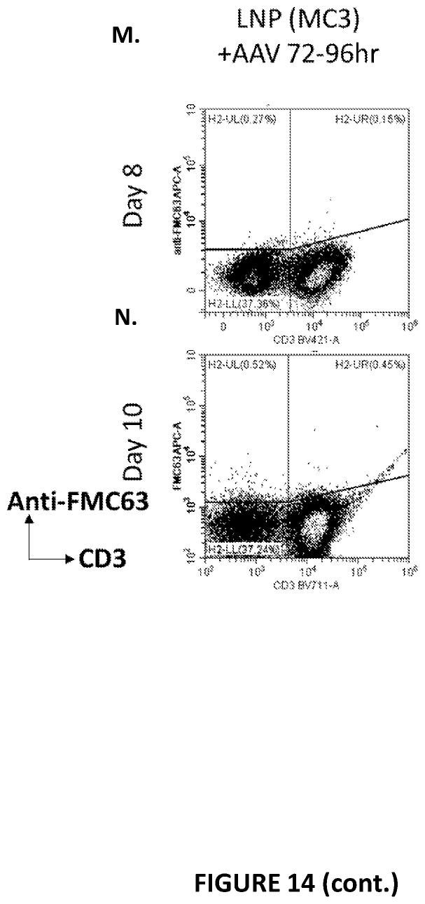

The CAR donor template was delivered to T cells via AAV transduction as described above. Analysis on day 3 post-transfection demonstrated that AAV addition between 24-48 hours after the LNP produced the highest frequency of CAR+/TCR− cells (˜6%), followed by 0-24 hours (2.2%), while the 48-72 hour group had yet to show expression by the day 3 time point (FIG. 14). Analysis on day 8 post-transfection demonstrated that the addition of AAV at 0-24 hours or 24-48 hours post LNP produced comparable CAR+/TCR− frequencies of ˜11-12%, and ˜40% CD3 KO overall. Analysis on day 10 post-transfection showed a further increase in CAR+/TCR− cells, with comparable frequencies between the 0-24 hour (21%) and 24-48 hour (21%) groups. The 48-72 hour group showed significantly less CAR+/TCR− cells (5%), and the 72-96 hour group showed less than 1% CAR+/TCR− cells.

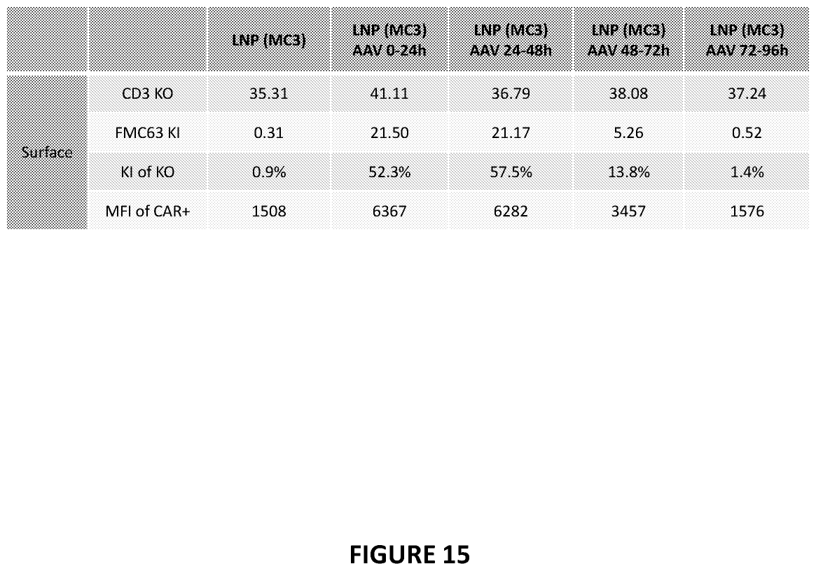

Overall analysis of the CAR T cell populations on day 10 post LNP transfection (˜40% CD3 KO) showed that the optimal time point for AAV addition to generate greater than 50% knock-in (KI) of CD3 knockout (KO) was between 0-48 hours post-LNP transfection (FIG. 15). Accordingly, these experiments demonstrated that the time of addition of the AAV was optimally within 48 hours of LNP transfection. Addition of AAV at a time greater than 48 hours post-transfection generated significantly fewer CAR+/TCR− T cells in the population.

In summary, this experiment demonstrated the production of CAR T cells using LNPs to deliver nuclease mRNA, and AAV to deliver the CAR donor template. Analysis of the CAR T cell populations showed a similar level of both CD3 knockout and CD4:CD8 ratios in populations produced using electroporation or the MC3 LNP. Surprisingly, the inclusion of an apolipoprotein (ApoE) with the LNP transduction resulted in a greater than a 2-fold increase in the production of CD3 knockout cells. Further analysis demonstrated that the time of transduction with the AAV post-LNP transfection was optimally within the first 48 hours post-transfection, generating greater than 50% knock-in of the CD3 knockout population.

### Example 6

### Analysis of CAR T Cell Function for CAR T Cells Generated Using LNP Transfection and AAV Transduction

### 1. Lipid Nanoparticle Formulations

The lipid materials used for the formulation of LNP in this experiment consisted of DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80° C. and thawed by heating to 50° C. in heat block. The lipid mix was removed from the heat source and vortexed immediately before use in formulation.

The mRNA material coding for the ARCUS TRC nuclease in this experiment consisted of a clean cap 1 structure with substitution of uridine nucleotides with pseudo-uridine. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

In this example, an apheresis sample was obtained from a healthy, informed, and compensated donor, and the T cells were enriched using the CD3 positive selection kit II in accord with the manufacturer's instructions (Stem Cell Technologies). T cells were activated using Immunocult T cell stimulator (anti-CD2/CD3/CD28—Stem Cell Technologies) in X-VIVO 15 medium (Lonza) supplemented with 5% fetal bovine serum and 10 ng/ml IL-2 (Gibco). To assess efficacy of LNP formulations to deliver mRNA encoding a TRC nuclease to edit at the TCR locus and reduce TCR on the cell surface, the LNP formulations were added to human donor T cells. Specifically, after 3 days of stimulation, cells were collected and samples of 0.5e6 cells were treated with 1 μg/mL of ApoE then transfected with 2 μg/mL of LNP formulation with mRNA coding for TRC nuclease.

To assess the optimal time point for CAR gene insertion and to produce a potent CAR T cell capable of targeted CD19+ cell killing, AAV6-7206 carrying an anti-CD19 construct was added to cells in culture to provide a CD19 CAR (FMC63) donor template. AAV6-7206 was added at different time points post LNP nuclease transfection. Specifically, at 0-24, 24-48 or 48-72 hours post LNP transfection, the AAV6-7206 carrying an anti-CD19 construct was added to cells. On day 10, CAR T cells were collected and placed in a co-culture assay with Raji cells (B cell lymphoma line, Burkitt's Lymphoma) as targets. The co-culture contained 10,000 FMC63+ CAR T cells and 10,000 Raji cells in a final volume of 200 μL.

### 3. Analysis

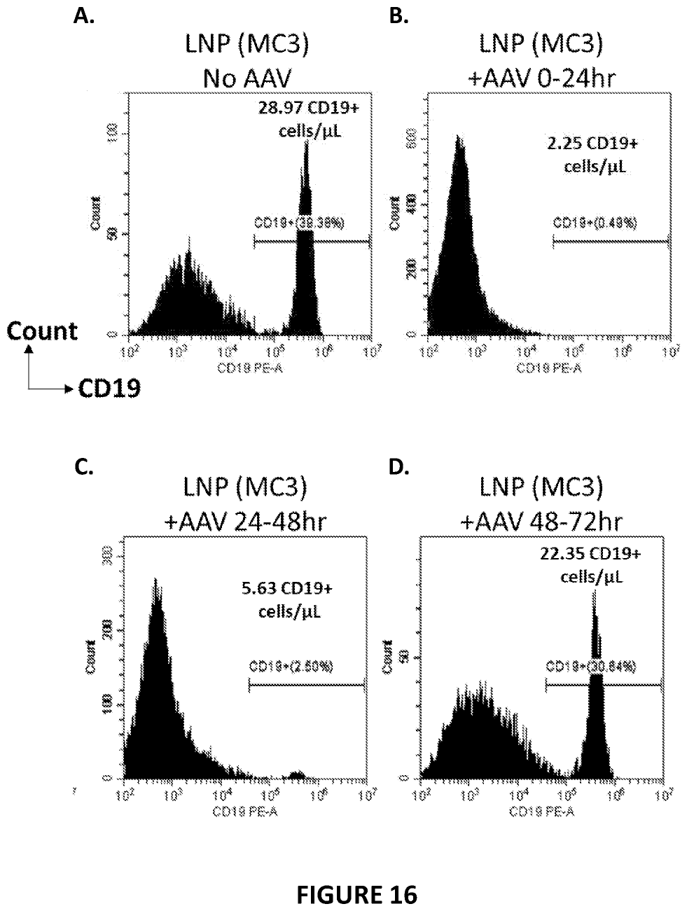

Cell phenotype was assessed by flow cytometry 16 hours post co-culture setup. Cells were collected, stained with Ghost Dye Violet 510 (Tonbo Biosciences), anti-human CD3-BV711, clone UCHT1 (BioLegend), anti-human CD4-FITC, clone OKT4 (BioLegend), anti-human CD8-BV421, clone RPA-T8 (BioLegend), and anti-FMC63 recombinant antibody-AF647, clone VM16 (BioLegend) washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live. CD19 expression was gated from live cell population. The results of these experiments are summarized in FIG. 16.

### 4. Results

At 16 hours of co-culture, CAR T cells generated using MC3 LNP mRNA transfection, followed by AAV transduction, were functional and capable of killing CD19+ Raji cells in the co-culture assay. The cytotoxic action of the CAR T cells was potent, with cells generated by MC3 LNP transfection and addition of AAV at 0-24 hours killing 99% of Raji cells, and cells generated by MC3 LNP transfection and addition of AAV at 24-48 hours killing 94% of Raji cells (FIG. 16). The addition of AAV at 48-72 hours post-LNP transfection produced a detectable but reduced cytotoxic effect on the CD19+ Raji cells. As expected, T cell populations that were transfected with the MC3 LNP but not transduced with AAV had little effect on the CD19+ Raji cell population in co-culture, as no CAR was expressed on these T cells.

In summary, co-culturing of CD19+ Raji cells with CAR T cells generated using the MC3 LNP with and without AAV addition at different times post-transfection demonstrated a clear and potent killing of CD19+ cells within 16 hours. Transduction of T cells with AAV within 0-24, 24-48, or 48-72 hours post-LNP transfection was capable of producing active CAR T cell populations, although potency was significantly higher in the 0-24 and 24-48 hour groups.

### Example 7

### LNPs to Deliver Nuclease mRNA for Production of CAR T Cells

### 1. Lipid Nanoparticle Formulations

The lipid materials used for the formulation of LNP in this experiment consisted of DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80° C. and thawed by heating to 50° C. in heat block. The lipid mix was removed from the heat source and vortexed immediately before use in formulation.

The mRNA material coding for the ARCUS TRC nuclease in this experiment consisted of a clean cap 1 structure with substitution of uridine nucleotides with pseudo-uridine. mRNA was stored at −80° C. and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

In this example, an apheresis sample was obtained from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum-free medium. Samples of 1e6 cells were electroporated with 1 μg of mRNA, coding for TRC nuclease, consisting of a clean cap 1 structure with substitution of uridine nucleotides with pseudo-uridine.

Within 10 minutes of electroporation, some cell samples were transduced by adding AAV-7206, carrying an anti-CD19 construct, to the culture. Other samples of 0.5e6 cells were treated with 1 μg/mL of ApoE then transfected with 2 μg/mL of LNP formulation with mRNA coding for TRC nuclease. At the time of LNP addition, some cell samples were transduced by adding AAV-7206, carrying an anti-CD19 CAR construct, to the culture. Specifically, at different time points post LNP transfection (0-96 hours), AAV containing CAR-T (Anti-CD19) construct was added to cells in culture to determine the optimal time for CAR gene insertion to produce a potent CAR T-cell capable of targeted CD19+ cell killing. After 4 hours in serum-free medium the culture was supplemented with complete medium.

### 3. Analysis

Cell phenotype was assessed by flow cytometry. On days 4, 7 and 12, cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

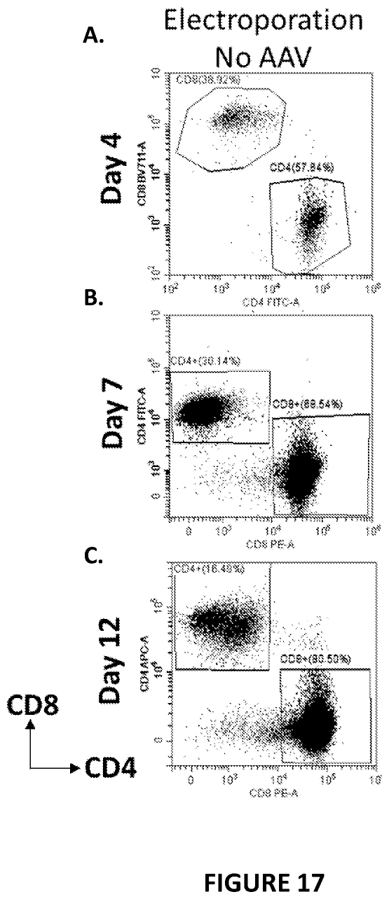

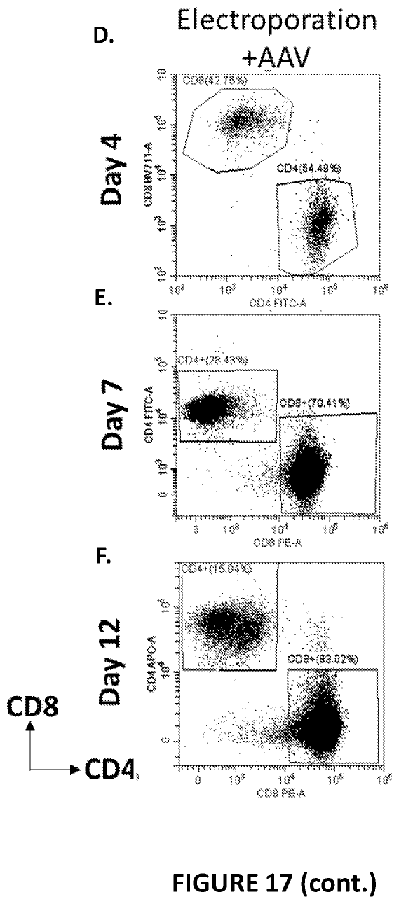

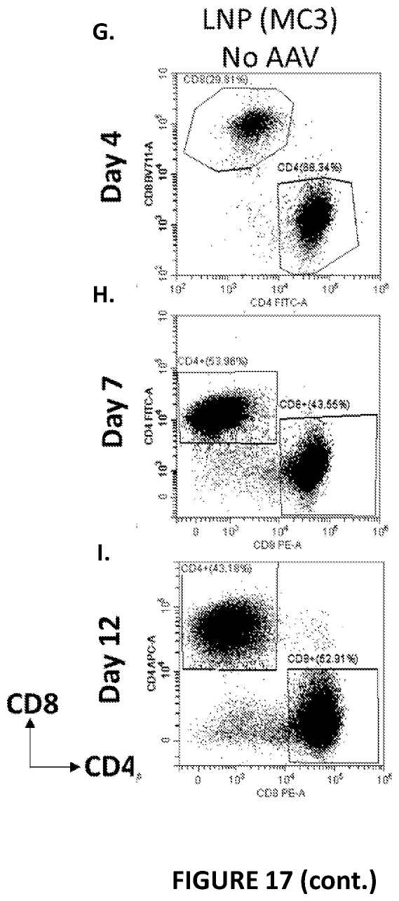

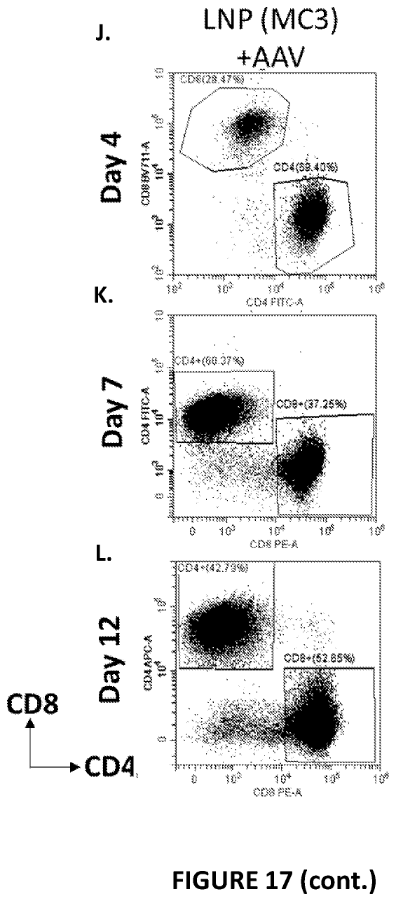

Analysis of CD4 and CD8 populations from days 4-12 following either electroporation or LNP transfection (with or without AAV transduction) demonstrated that electroporation elicited a low CD4:CD8 ratio in which, by day 12, the CD4+ population was less than 20% and the CD8+ population was greater than 80% (1:4 ratio CD4:CD8; FIG. 17). By contrast, through day 12, the LNP-transfected group (with or without AAV) demonstrated a more consistent CD4:CD8 ratio in which, by day 12, the CD4+ population was approximately 45% and the CD8+ population accounted for approximately 55% (1:1.2 ratio CD4:CD8). Thus, transfection of nuclease mRNA using LNPs resulted in a more even distribution of CD4+ and CD8+ cells in the CAR T cell population after 12 days of culture, whereas the use of electroporation resulted in populations skewed toward CD8+ cells. This was true whether the T cells were transduced with AAV or not.

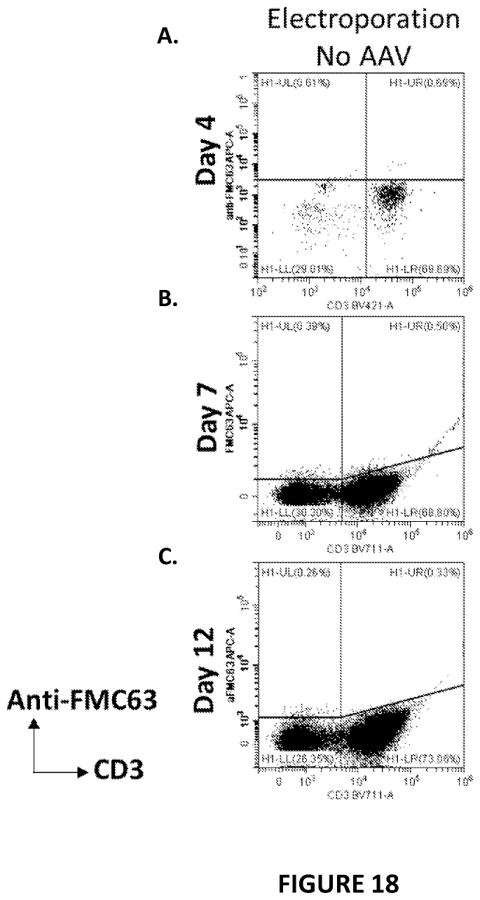

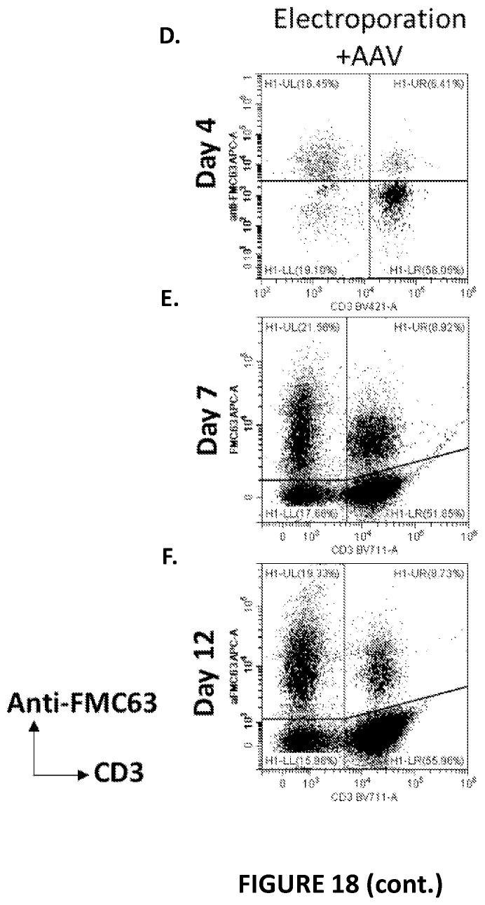

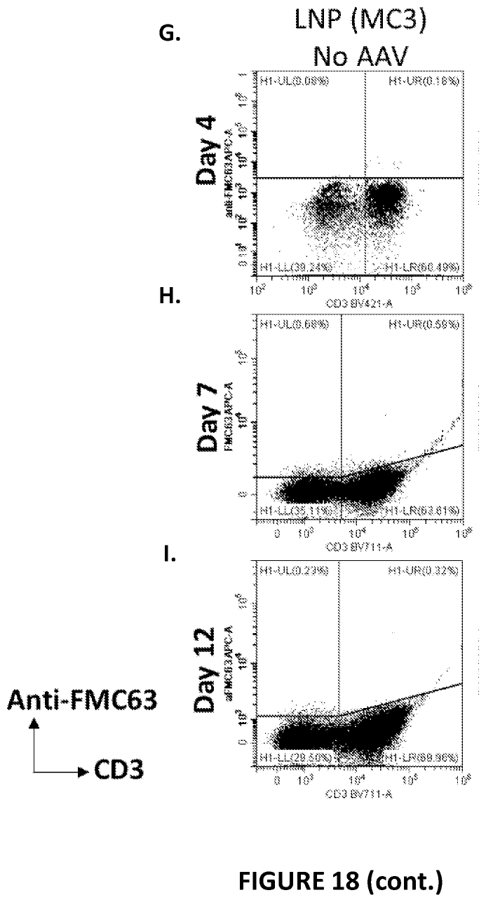

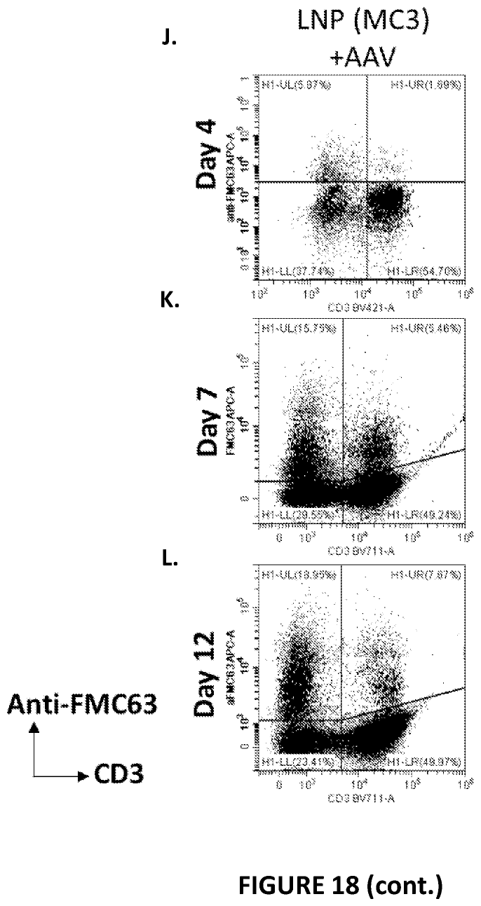

Analysis of CD3−/CAR+ populations from days 4-12 following either electroporation or LNP transfection (with or without AAV) demonstrated that both the electroporation and LNP groups generated a significant number of CD3−/CAR+ T cells (FIG. 18). The use of electroporation for mRNA transfection resulted in 21% CD3−/CAR+ cells on day 7, with a slight reduction to 19% by day 12, while the LNP-transfected group showed a progressive increase from 16% CD3−/CAR+ on day 7 to 19% on day 12.

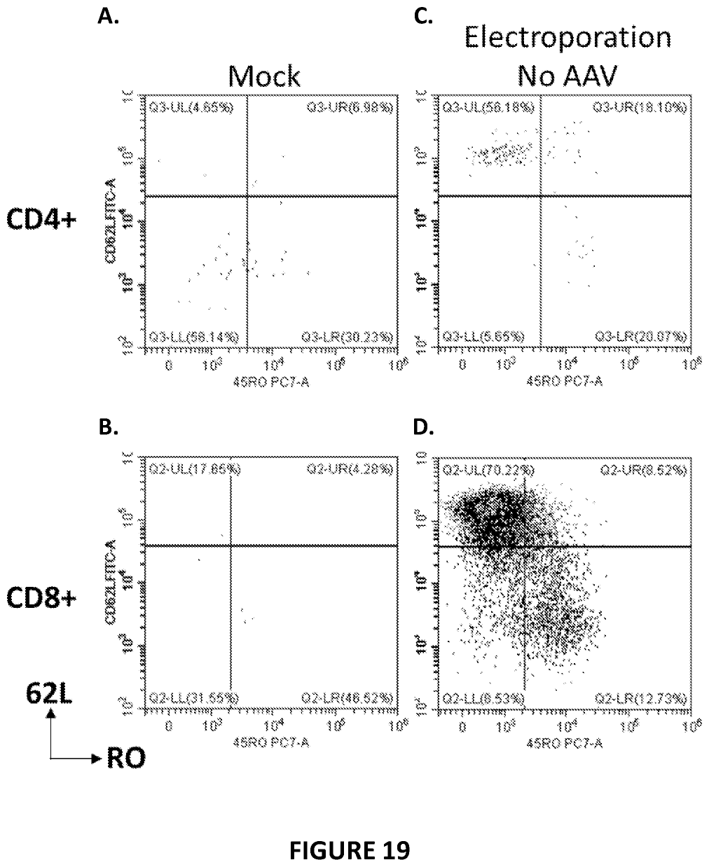

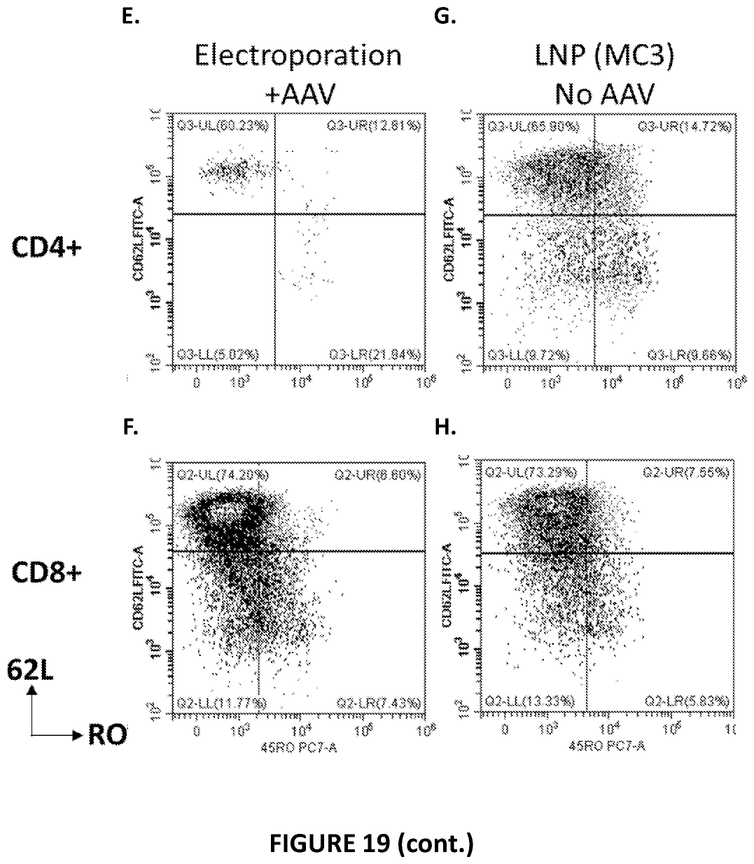

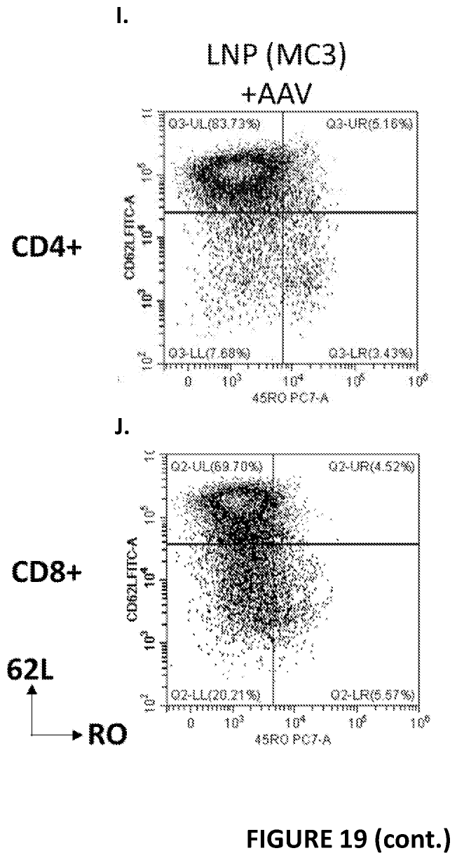

T cell populations were evaluated for memory phenotype 12 days post-transfection by electroporation or LNP (with or without AAV). For cells that were transduced with AAV, analysis for CD4+ memory (CD3−/CD4+/62L+/RO−) and CD8+ memory (CD3-/CD8+/62L+/RO−) phenotype demonstrated that both the electroporated and LNP-transfected groups generated a significant number of CD8+ memory phenotype cells (approximately 74% and 70%, respectively) on day 12. (FIG. 19). However, for cells transduced with AAV, electroporation resulted in a lower frequency of CD4+ memory phenotype cells (approximately 60%) compared to the LNP-transfected group (approximately 84%) by day 12.

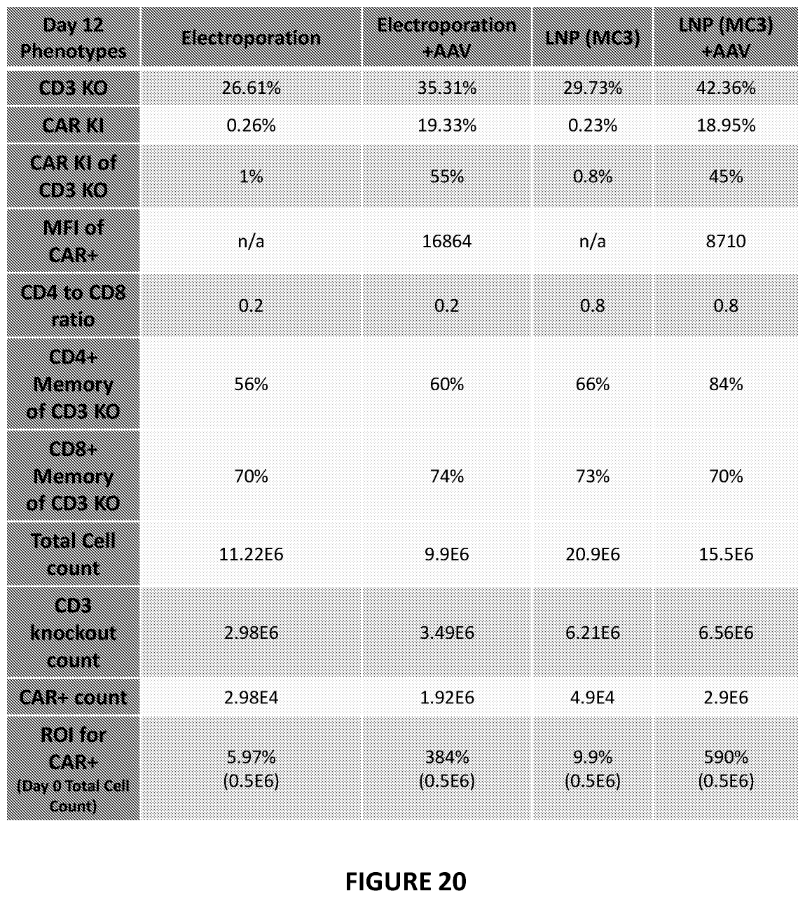

A table summarizing the phenotypes observed in the T cell populations at day 12 post-transfection is provided as FIG. 20. The electroporation and LNP groups, with the addition of AAV carrying the CAR, generated significant CD3−/CAR+ populations (35% and 42%, respectively). The LNP group produced a more balanced CD4:CD8 population (ratio of 0.8) compared to the electroporated group (ratio of 0.2), as well as a higher population of CD4+ central memory phenotype cells (84% vs 60%). The return on investment (ROI) in generating CD3−/CAR+ cells from an initial donor T cell population was approximately 1.5-fold greater post-LNP transfection when compared to electroporation (590% vs 384%, respectively).

In summary, this experiment evaluated several characteristics of CAR T cell populations generated using electroporation or LNPs for transduction of nuclease mRNA, in combination with AAV transduction for delivery of a CAR donor template. These population characteristics included CD4:CD8 ratios, memory phenotype, and overall return on investment, each of which is an important aspect of a CAR T cell clinical product. Although electroporation is the current gold standard for T cell transfection, the results of this study surprisingly demonstrated that the use of LNPs produced CAR T cell populations with several advantageous characteristics that could not have been anticipated. Through day 12, the LNP-transfected group exhibited a more balanced CD4:CD8 ratio. By comparison, the electroporated group exhibited a CD4:CD8 ratio that was largely skewed towards CD8+ cells. Further analysis of the CD4+ population also demonstrated that the LNP-transfected group preserved the advantageous central memory phenotype to a greater degree than the electroporated CAR T cell group. Furthermore, the return on investment (ROI) in generating CD3−/CAR+ cells from an initial donor T cell population showed an approximately 1.5-fold greater return post-LNP transfection compared to the use of electroporation. These results clearly demonstrated that the use of LNPs for mRNA delivery was superior for the production of a CAR T cell product relative to the use of electroporation.

### Example 8

### Use of LNPs to Deliver Repeat Dosing of Nuclease mRNAs for Production of Increased Target Gene Knockout in Donor T Cells

### 1. Lipid Nanoparticle Formulations

In these studies, donor T cells were transfected with mRNA encoding an engineered meganuclease having specificity for a recognition sequence within the human beta-2 microglobulin gene. This engineered meganuclease is referred to as B2M13-14.479 and has previously been shown to knockout cell-surface expression of B2M on the surface of T cells (see, WO 2017112859).

The lipid materials used for the formulation of LNP comprised DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80 C and thawed by heating to 50 C in heat block. Lipid mix was taken off heat and vortexed immediately before use in formulation. The mRNA material coding for the B2M nuclease consisted of a clean cap 1 structure with substitution of uridine nucleotides with pseudo-uridine. mRNA was stored at −80 C and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

In this example the formulation was added to human donor T cells on day 0 and day 3 to asses efficacy of LNP formulations to deliver repeatable doses of a nuclease mRNA. The formulation of mRNA encoding a B2M nuclease to edit at the B2M locus and reduce B2M on the cell surface, measured by B2M staining and flow cytometry analysis.

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum-free medium. Samples of 1e6 cells were electroporated with 1 μg of mRNA, coding for B2M nuclease, consisting of a clean cap 1 structure with substitution of uridine nucleotides with pseudo-uridine. Other samples of 1e6 cells were treated with 1 μg/mL of ApoE then transfected with 1 μg/mL of LNP formulation with mRNA coding for B2M nuclease. 3 days post first transfection, 1 μg/mL of LNP formulation with mRNA coding for B2M nuclease was added to half of wells which received transfection on day 0.

### 3. Analysis

Flow cytometry was used to assess cell phenotype of cells at days 7. Cells were collected and stained with Ghost Dye Violet 510 (Tonbo Biosciences), anti-human B2-microglobulin, clone 2M2 (Biolegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

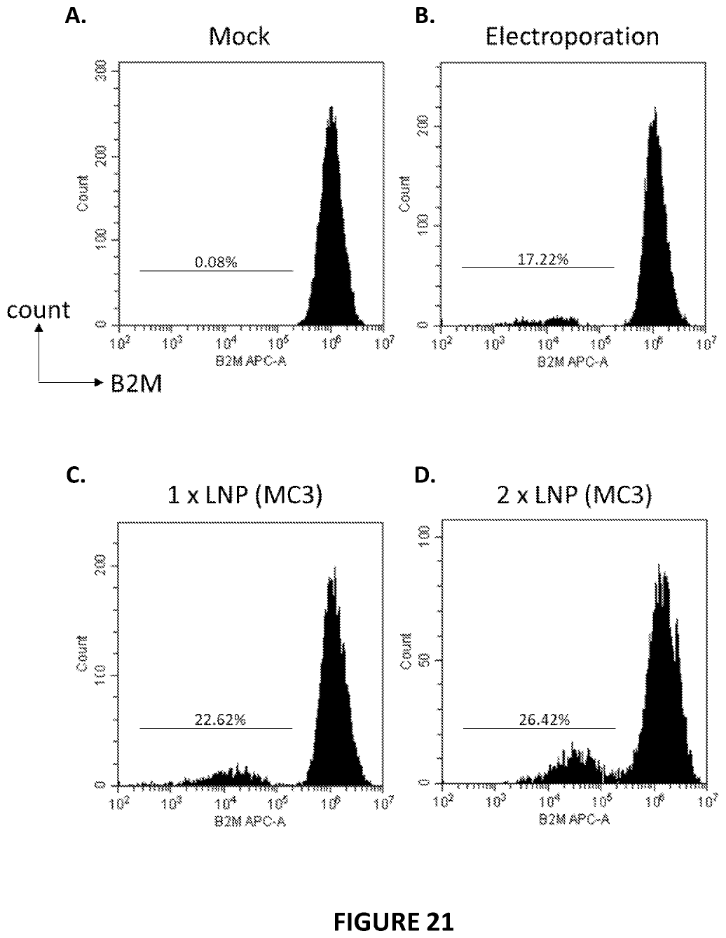

Analysis of B2M populations from day 7 following either electroporation or LNP transfection (with LNP transfection on day 0 only, or repeated LNP dosing on day 0 and day 3) demonstrated that repeat dosing elicited an increase in B2M knockout at day 7 (22.62% versus 26.42%) (FIG. 21).

In summary, this study evaluated repeated dosing of B2M nuclease mRNA using LNPs. Due to the complex processing of electroporation, repeat dosing is not a plausible option. Therefore, these results clearly show that the use of LNPs for repeated mRNA delivery results in increased gene knockout compared to single administration and demonstrates the LNPs flexibility to process changes compared to electroporation.

### Example 9

### Use of LNPs to Deliver Multiple Nuclease mRNAs for Production of Dual Gene KO CAR T Cells

### 1. Lipid Nanoparticle Formulation

The lipid materials used for the formulation of LNP comprised DLin-MC3-DMA, Cholesterol, DSPC, and DMG-PEG dissolved at a 50:38.5:10:1.5 molar ratio in ethanol at a total lipid concentration of 30 mM (high N:P=8). The mix of lipids was stored at −80 C and thawed by heating to 50 C in heat block. Lipid mix was taken off heat and vortexed immediately before use in formulation. The mRNA material coding for the TRC nuclease consisted of a clean cap 1 structure with uridine substitution of pseudouridine. The mRNA material coding for the B2M nuclease consisted of a clean cap 1 structure substitution of uridine nucleotides with pseudo-uridine. mRNA was stored at −80 C and thawed at room temperature. Once thawed, the mRNA was diluted to 0.2 mg/mL in a sucrose/Tris/Acetate buffer at pH=4.0. Microfluidic mixing of the mRNA and lipid solutions at a 3:1 ratio into an exchange buffer (sucrose/Tris/Acetate pH=8.0) via Precision Nanosystems SPARK mixer was performed. Final solution in the exchange buffer reservoir was collected and analyzed for physical characteristics such as size, PDI, and zeta potential, as well as for encapsulation efficiency.

### 2. T Cell Culture and Transfection

The formulation was added to human donor T cells to asses efficacy of LNP formulations to deliver mRNA encoding a TRC and/or B2M nuclease to edit at the TCR and/or B2M locus and reduce TCR and/or B2M on the cell surface, measured by CD3 and B2M staining and flow cytometry analysis. Addition of AAV carrying a CAR T (Anti-CD19) construct was added at same time as LNP nuclease transfection and CAR gene insertion was assessed.

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum-free medium. Samples of 1e6 cells were electroporated with 1 μg of mRNA, coding for TRC nuclease, consisting of a clean cap 1 structure with uridine substitution of pseudouridine. Within 10 minutes of electroporation, some cell samples were transduced by adding AAV-7206, carrying an anti-CD19 construct, to the culture. Other samples of 1e6 cells were treated with 1 μg/mL of ApoE then transfected with 1 μg/mL of LNP formulation with mRNA coding for TRC nuclease and 1 μg/mL of LNP formulation with mRNA coding for B2M nuclease. At the time of LNP addition, cell samples were also transduced by adding AAV-7206, carrying an anti-CD19 CAR construct, to the culture. After 4 hours in serum-free medium the culture was supplemented with complete medium.

### 3. Analysis

Flow cytometry was used to assess cell phenotype of cells at day 7. Cells were collected and stained with Ghost Dye Violet 510 (Tonbo Biosciences), anti-human B2-microglobulin, clone 2M2 (Biolegend), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

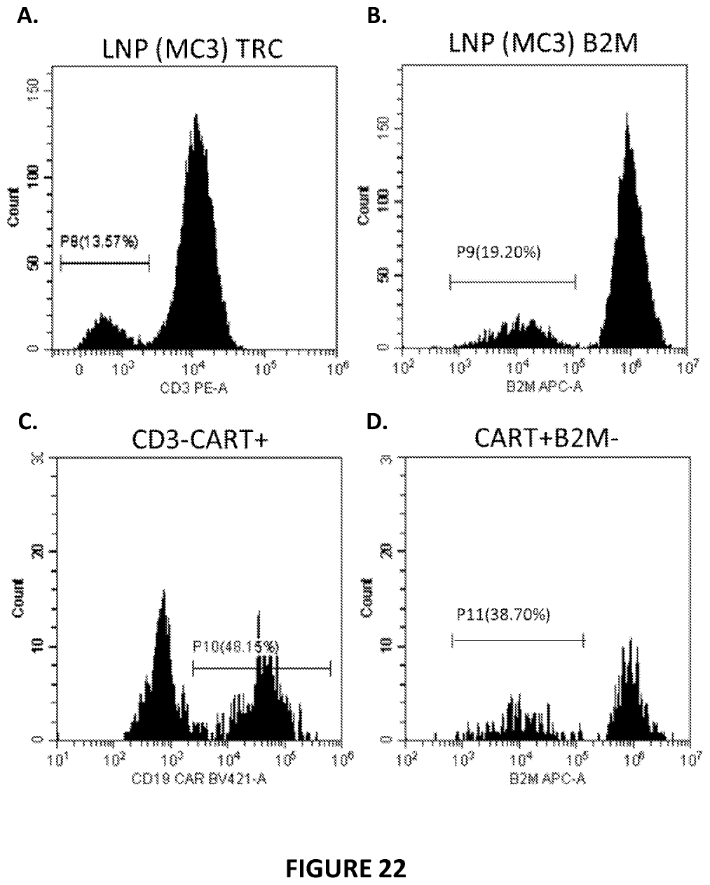

Analysis of B2M, TRC, and CAR T populations on day 7 following LNP transfection (with TRC and B2M nucleases), as well as AAV CAR transduction, demonstrated TCR knockout (13.57%), B2M knockout (19.20%), and CAR knock-in of CD3− cells (48.15%), in which CAR+CD3− cells also had 38.70% B2M knockout (FIG. 22).

In summary, this example evaluated the ability to generate a CAR T cell population with dual gene knockouts of TCR and B2M using LNPs for the delivery of nuclease mRNA. These results provide proof-of-concept that LNPs delivery of mRNA is useful for the production of a CAR T cell product with multiple desired gene knockouts.

### Example 10

### Use of LNPs to Deliver Nuclease mRNA for Production of CAR T Cells

### 1. Lipid Nanoparticle Formulation

The lipid materials used for the formulation of LNP consists of DLin-MC3-DMA, Cholesterol, a phospholipid (DSPC, DOPC, or DOPE), and DMG-PEG (2000 or 5000) dissolved at varying molar ratios in ethanol at a total lipid concentration of 15 mM (Formulated at constant N:P=8). The mix of lipids was stored at −80° C. and thawed by heating to 50° C. in heat block. Lipid mix was taken off heat and vortexed immediately before use in formulation. The mRNA material coding for the TRC 1-2L.1592 nuclease consisting of a clean cap 1 structure with substitution of uridine nucleotides with pseudo-uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Only formulations with encapsulation >80% and remained stable (no visible aggregation) over 2 days at 4° C. were used in the transfection experiment.

The formulation was added to human donor T-cells to asses efficacy of LNP formulations to deliver mRNA encoding a TRC nuclease to edit at the TCR locus and reduce TCR on the cell surface, measured by CD3 staining and flow cytometry analysis.

### 2. T Cell Culture and Transfection

In this study, an apheresis sample was drawn from a healthy, informed, and compensated donor, and the T cells were enriched using the CD3 positive selection kit II in accordance with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum-free medium. Samples of 1e6 cells were electroporated with 1 μg of mRNA, coding for TRC nuclease, consisting of a clean cap 1 structure with substitution of uridine nucleotides with pseudo-uridine. Other samples of 1e5 cells were treated with 1 μg/mL of ApoE then transfected with 2 μg/mL of LNP formulation with mRNA coding for TRC nuclease. After 4 hours in serum-free medium the culture was supplemented with complete medium.

### 3. Analysis

Flow cytometry was used to assess cell phenotype of cells at days 4, 7, and 10. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences), and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

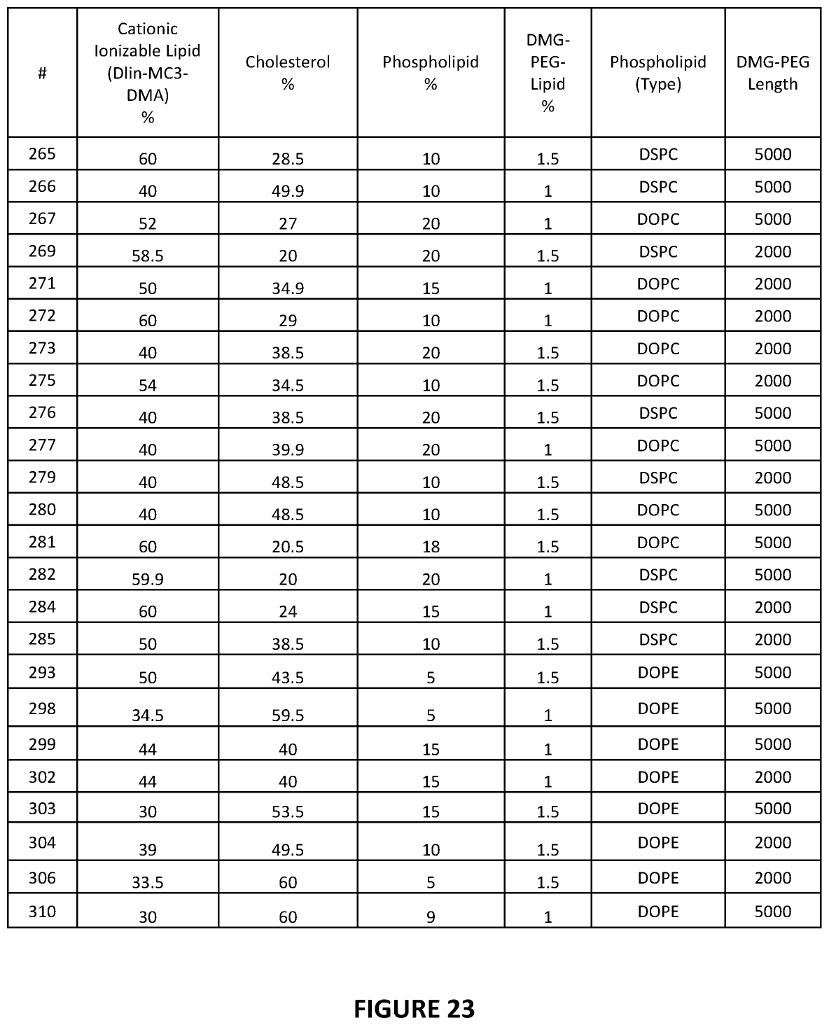

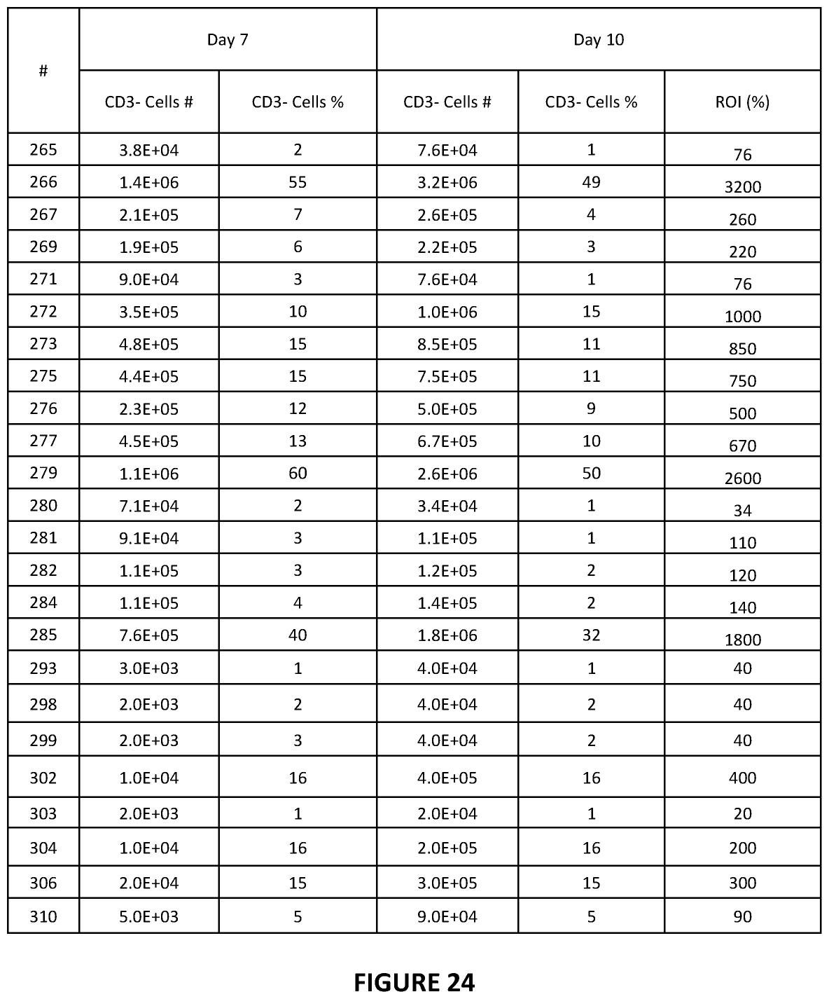

This study evaluated several characteristics of LNPs that drive potency of nuclease mRNA in T cell transfection. Formulations were derived using SAS JMP DOE Software in which a cationic lipid (20-60%), cholesterol (20-60%), phospholipid (5-20%), DMG-PEG (0.1-1.5%), with phospholipid type (DSPC, DOPC, or DOPE), and PEG length (2000 or 5000) were factors treated as a mixture in which all components equaled 1 or 100%. The formulations were generated at a constant N:P of 8. FIG. 23 provides a table summarizing the formulations screen in T-cell transfection, which passes formulation QC. As shown in FIG. 24, the percent CD3− cells and return on investment (ROI) in generating CD3− cells from an initial donor T cell population ranged from 1% to 60% KO and 76% to 3200% ROI depending on the formulation lipid ratio, phospholipid, and PEG type.

Through extensive analysis with custom mixture design of experiments (DOEs) via JMP statistical software, it was unexpectedly determined that subtle changes in LNP composition drive dramatic changes in the potency of transfection in human derived T cells. Furthermore, although the original formulation (#285) at 50:38.5:10:1.5 of MC3:Chol:DSPC:PEG2000 performed well with the TCR KO (CD3−) and return on investment (ROI) of 32% KO and 1800% ROI, we further found that increasing the cholesterol seen in the formulation (#266 and 279) resulted in an increase of TCRKO (CD3−), 49% KO and 50% KO, and a ROI of 3200% and 2600%. Furthermore, although it has been reported that DOPE increases fusogenicity and increases transfection ability, these experiments found that DSPC remained to be the optimal phospholipid for T cell transfection. In investigating the PEG length, we found that neither 2000 or 5000 dramatically changed transfection ability, however, formulations with lower PEG % (0.1 to 0.5%) showed signs of instability when using 2000 versus with 5000. These results showed that subtle changes in LNP composition can dramatically alter mRNA delivery and that rational design of LNPs for improved cell transfection is unlikely.

### Example 11

### Use of LNPs to Deliver Nuclease mRNA for Production of CAR T Cells

### 1. Lipid Nanoparticle Formulation

The lipid materials used for the formulation of LNP consists of an ionizable/non-ionizable cationic lipid, cholesterol, a phospholipid (DSPC, DOPC, or DOPE), and DMG-PEG (2000 or 5000) dissolved at varying molar ratios in ethanol at a total lipid concentration of 15 mM (Formulated at constant N:P=8). The mix of lipids was stored at −80° C. and thawed by heating to 50° C. in heat block. The lipid mix was taken off heat and vortexed immediately before use in formulation. The mRNA material coding for the engineered TRC nuclease included a clean cap 1 structure with substitution of uridine nucleotides with pseudo-uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Only formulations with encapsulation >80% and remained stable (no visible aggregation) over 2 days at 4° C. were used in the transfection experiment.

The formulation was added to human donor T-cells to asses efficacy of LNP formulations to deliver mRNA encoding a TRC nuclease to edit at the TCR locus and reduce TCR on the cell surface, measured by CD3 staining and flow cytometry analysis.

### 2. T Cell Culture and Transfection

In this study, an apheresis sample was drawn from a healthy, informed, and compensated donor, and the T cells were enriched using the CD3 positive selection kit II in accordance with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) of fetal bovine serum and 10 ng/ml IL-2 (CellGenix). After 3 days of stimulation, cells were collected, washed, and resuspended in serum-free medium. Samples of 1e6 cells were electroporated with 1 μg of mRNA, coding for the engineered TRC nuclease, which included a clean cap 1 structure with substitution of uridine nucleotides with pseudo-uridine. Other samples of 5e5 cells were treated with 1 μg/mL of ApoE then transfected with 0, 1.0, 2.5, or 5 μg/mL of LNP formulation with mRNA coding for the engineered TRC nuclease. Addition of AAV was assessed at varying doses (0K, 5K, 25K, or 125K multiplicity of infection (MOI)) in serum free media as well as time of addition (−12 h), during (0 h), or after (12 h) from LNP addition.

### 3. Analysis

Flow cytometry was used to assess cell phenotype at days 4, 7, and 10. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco), and analyzed on CytoFLEX LX (Beckman Coulter). Post-run analysis was completed using CytExpert software (Beckman Coulter). Cells were gated on FSC vs SSC scatter followed by singlets then live.

### 4. Results

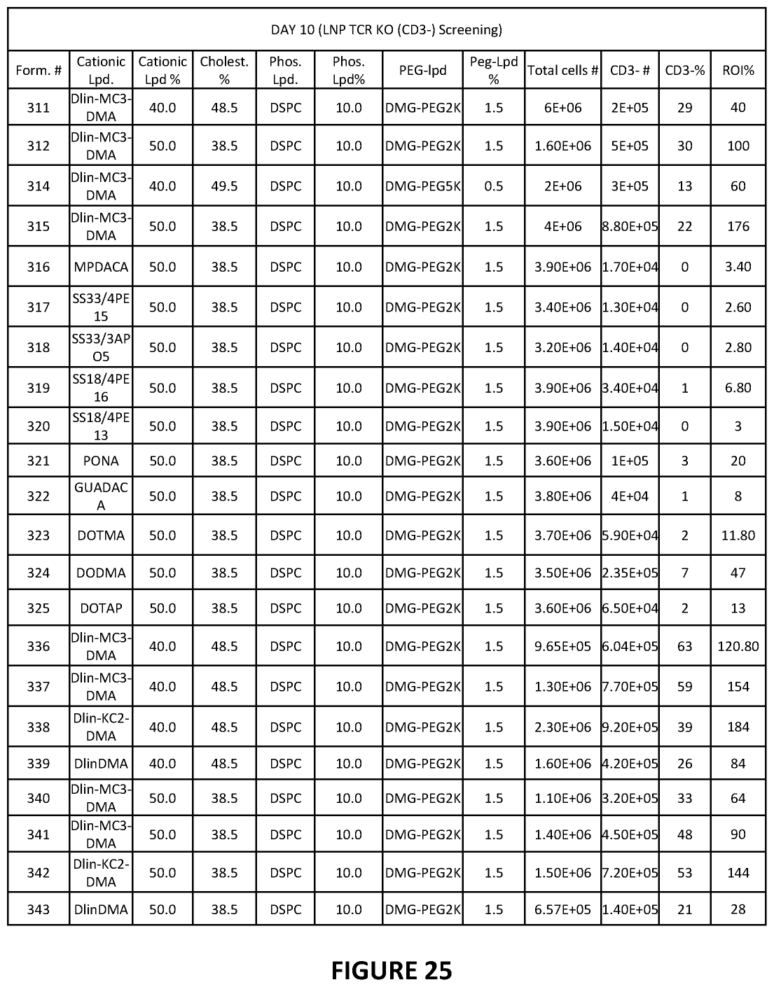

This study evaluated several cationic lipids for LNP formulation along with optimization of AAV time of addition and dosing. Formulations were derived using molar ratios of cationic lipid:cholesterol:phospholipid:PEG-lipid experimentally tested, in which the cationic lipid was replaced with other cationic ionizable and non-ionizable lipids. The formulations were generated at a constant N:P of 8. The composition of each formulation and their efficacy in transfecting mRNA encoding a TRC nuclease to edit at the TCR locus and reduce TCR on the cell surface, measured by CD3 staining is provided in FIG. 25. The total number of cells, number of CD3− cells and CD3− percentage is shown. Through analysis of numerous cationic lipids (with varying hydrophobic tails and hydrophilic head groups) it was observed that lipids with the DLinDMA unsaturated tails (1,2-dilinoleoyl) had the strongest effect to transfection potency. Investigation of three DLinDMA based lipids (DLinDMA, DLin-KC2-DMA, and DLin-MC3-DMA) determined that the 1,2-dilinoleoyl group achieves >20% indels determined by the number and percentage of CD3− cells. All other lipids screened resulted in low <5% indels, besides DODMA (1,2-dioleyloxy), which achieved 7% indels and historically has achieved >10% indels. Furthermore, the substitution of the hydrophilic head group from DMA<KC2-DMA<MC3-DMA increases potency in the formulation based off 40:48.5:10:1.5, achieving 26%, 39%, 59% indels respectively. However, DMA<MC3-DMA<KC2-DMA increases potency in the formulation based off 50:38.5:10:1.5, achieving 21%, 48%, 53% indels respectively. This may be due to the specific endosomal pH and the differences in pKa between KC2 and MC3, and the percent of cationic lipid and cholesterol used in the two distinct formulations.

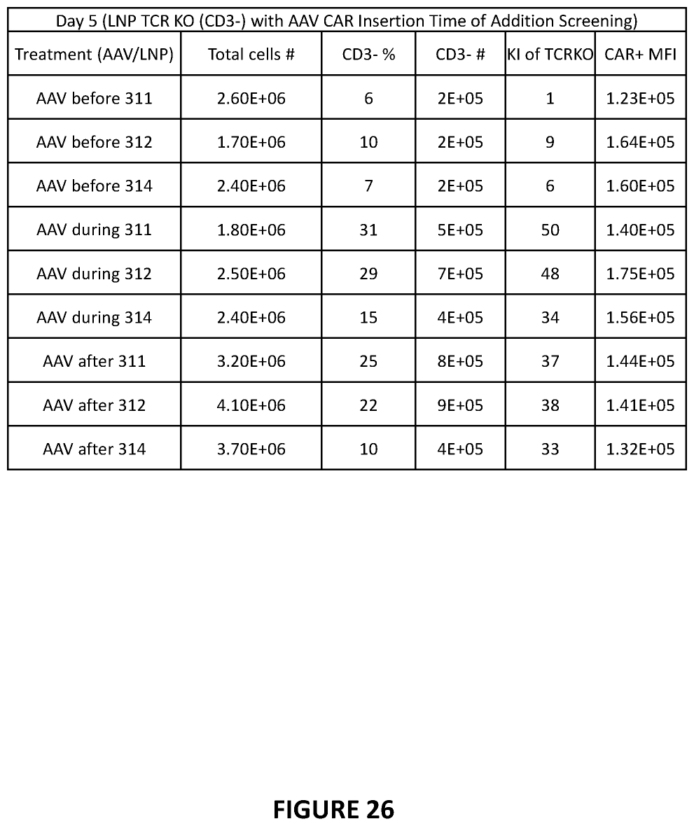

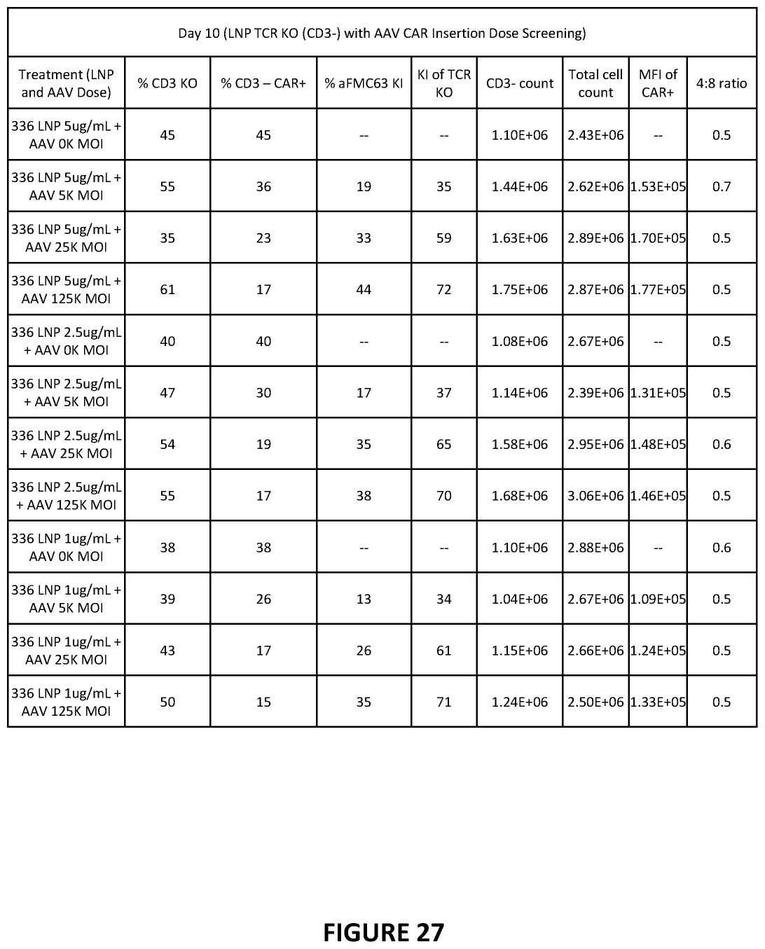

Analysis of the AAV time of addition shows that the AAV should be added at the time of LNP transfection or within 24 hrs after LNP addition to generate the highest number of CD3-CAR+ cells (FIG. 26). Furthermore, dose range finding of LNP and AAV demonstrates that LNP 336 is efficient at transfection at 1 μg/mL to 5 μg/mL, and AAV addition at 5 to 125 MOI increases LNP delivery resulting in increased CD3 knockout (FIG. 27). Furthermore, it is evident that there is a dose dependence on CAR T production as the dose of LNP and AAV increase.

### Example 12

### Use of LNPs to Deliver Nuclease mRNA for Production of CAR T Cells

### 1. Lipid Nanoparticle Formulation

This study evaluated the cationic lipid SS-OP (Bis[2-(4-{2-[4-(cis-9-octadecenoyloxy)phenylacetoxy]ethyl}piperidinyl)ethyl] disulfide) in LNP formulations used to transfect T cells with mRNA encoding an engineered nuclease to knockout the TCR locus. SS-OP is a cationic lipid containing a reductive sensitive disulfide bond as well as a self-degradable phenyl ester via thioesterification.

The lipid materials used for the formulation of the LNP consisted of SS-OP as the cationic lipid, Cholesterol, a Phospholipid (DSPC), and DMG-PEG (2000) dissolved at varying molar ratios in ethanol at a total lipid concentration of 15 mM (Formulated at constant N:P=8). The mix of lipids was stored at −80° C. and thawed by heating to 50° C. in heat block. Lipid mix was taken off heat and vortexed immediately before use in formulation. The mRNA material coding for the engineered TRC nuclease included a clean cap 1 structure with substitution of uridine nucleotides with pseudo-uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Only formulations with encapsulation >80% and remained stable (no visible aggregation) over 2 days at 4° C. were used in the transfection experiment.

The formulation was added to human donor T-cells to asses efficacy of LNP formulations to deliver mRNA encoding a TRC nuclease to edit at the TCR locus and reduce TCR on the cell surface, measured by CD3 staining and flow cytometry analysis.

### 2. T Cell Culture and Transfection

In this study, an apheresis sample was drawn from a healthy, informed, and compensated donor, and the T cells were enriched using the CD3 positive selection kit II in accordance with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) of fetal bovine serum and 10 ng/ml IL-2 (CellGenix). After 3 days of stimulation, cells were collected, washed, and resuspended in serum-free medium. Samples of 1e6 cells were electroporated with 1 μg of mRNA, coding for the engineered TRC nuclease, which included a clean cap 1 structure with substitution of uridine nucleotides with pseudo-uridine. Other samples of 5eE cells/mL were treated with 1 ug/mL of ApoE then transfected with 2.0 ug/mL of LNP formulation with mRNA coding for TRC nuclease.

### 3. Analysis

Flow cytometry was used to assess cell phenotype of cells at days 4, 7 and 10. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

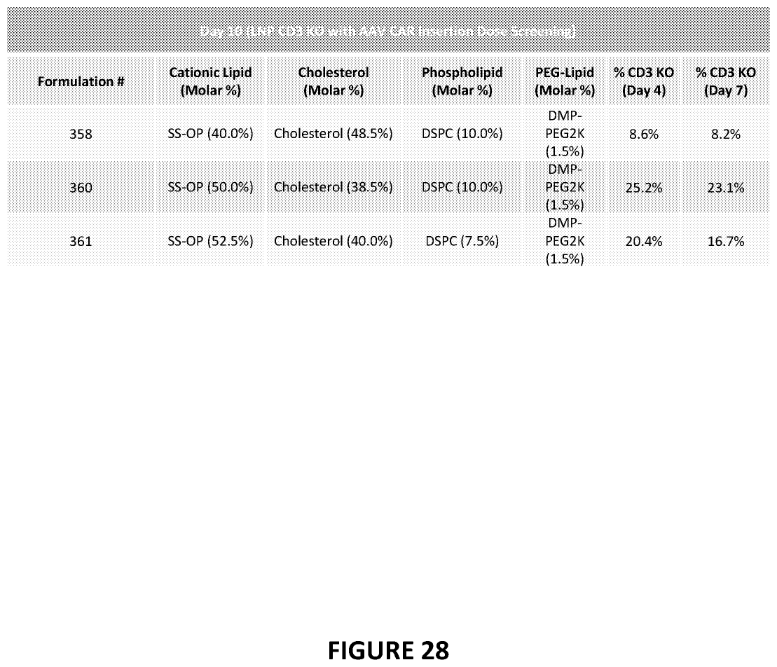

FIG. 28 provides a table summarizing the formulations screened in T cell transfection which passed formulation QC. The formulations were generated at a constant N:P of 8. FIG. 28 also provides data demonstrating the percent of CD3 knockout observed at day 4 and 7 post-transfection with nuclease mRNA.

By analyzing CD3 knockout as a measure of TCR gene inactivation, it was observed that formulations containing the SS-OP lipid were effective at delivering nuclease mRNA for gene knockout. Investigation of three formulation variants determined that the formulations 360, 361, and 358, having molar ratios of 50:38.5:10:1.5, 52.5:40:7.5:1.5, and 40:48.5:10:1.5, respectively, achieved 25.2%, 20.4%, and 8.6% indels, respectively, at 4 days post-transfection of the mRNA encoding the nuclease.

### Example 13

### Effect of Modified Nucleic Acids

### 1. Lipid Nanoparticle Formulation

The purpose of this experiment was to evaluate LNP formulations for delivering nuclease mRNA that included, or did not include, modified nucleic acids such as Pseudo UTP, in the production of CAR T cells.

The lipid materials used for the formulation of LNP included DLin-MC3-DMA, cholesterol, a phospholipid (DSPC), and DMG-PEG (2000) at a 50:38.5:10:1.5 molar ratio. Lipids were dissolved in ethanol at a total lipid concentration of 15 mM (Formulated at constant N:P=8). The mix of lipids was stored at −80° C. and thawed by heating to 50° C. in heat block. Lipid mix was taken off heat and vortexed immediately before use in formulation. The mRNA material coding for the TRC nuclease (previously described) included a clean cap 1 structure with unmodified uridine UTP (363 formulation) or substitution of pseudo-uridine (Pseudo UTP; 362 formulation). mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Only formulations with encapsulation >80% and remained stable (no visible aggregation) over 2 days at 4° C. were used in the transfection experiment.

The formulation was added to human donor T-cells to asses efficacy of LNP formulations to deliver mRNA encoding a TRC nuclease to edit at the TCR locus and reduce TCR on the cell surface, measured by CD3 staining and flow cytometry analysis.

### 2. T Cell Culture and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) of fetal bovine serum and 10 ng/ml IL-2 (CellGenix). After 3 days of stimulation, cells were collected, washed and resuspended in serum-free medium. Samples of 5eE cells/mL were treated with 1 ug/mL of ApoE then transfected with 2.0 ug/mL of LNP formulation with mRNA coding for TRC nuclease.

### 3. Analysis

Flow cytometry was used to assess cell phenotype of cells at days 4, 7 and 10. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

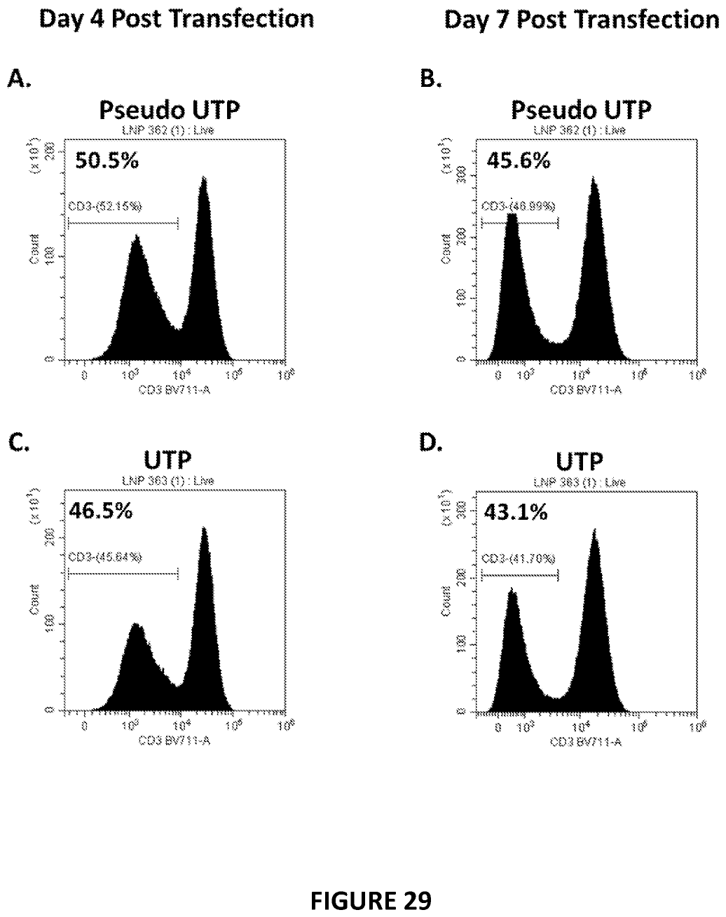

FIGS. 29A-29D illustrate the CD3 knockout efficiency of the 362 and 363 formulations at 4 days and 7 days post-transfection with mRNA encoding the TRC nuclease. The formulations were generated at a constant N:P of 8.

Each evaluated formulation, which included either UTP mRNA or Pseudo UTP mRNA, generated a high level of transfection potency, achieving a CD3 knockout efficiency of 46.5% (UTP) and 50.5% (Pseudo UTP), respectively, at 4 days post-transfection of nuclease mRNA, and a CD3 knockout efficiency of 43.1% (UTP) and 45.6% (Psuedo UTP), respectively, at 7 days post-transfection. Therefore, this data demonstrates that modified or unmodified mRNA can be used in LNP formulations for nuclease mRNA transfection of T cells.

### Example 14

### Use of LNPs in Presence of Serum Conditions to Deliver Nuclease mRNA for Production of CAR T Cells

### 1. Lipid Nanoparticle Formulation

The purpose of this experiment was to evaluate LNP formulations for delivering mRNA in the presence of serum for the production of CAR T cells. The lipid materials used for the formulation of LNP included DLin-MC3-DMA, cholesterol, DSPC, and DMG-PEG (2000) at a 40:48.5:10:1.5 molar ratio dissolved in ethanol at a total lipid concentration of 15 mM (formulated at constant N:P=8). The mix of lipids was stored at −80° C. and thawed by heating to 50° C. in heat block. Lipid mix was taken off heat and vortexed immediately before use in formulation. The mRNA material coding for the TRC nuclease including a clean cap 1 structure with unmodified uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Formulation was stored frozen (−80° C.) at 1 mg/ml in 250 mM sucrose in PBS. Formulation was thawed at room temperature and diluted to desired concentration in PBS before addition to cell culture media.

The formulation was added to human donor T cells to asses efficacy of LNP formulations to deliver mRNA encoding a TRC nuclease to edit at the TCR locus and reduce TCR on the cell surface, measured by CD3 staining and flow cytometry analysis.

### 2. T Cell Culture and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in varying degrees of serum supplemented medium (0-5%). Samples of 5e5 cells/mL were treated with 1 ug/mL of ApoE then transfected with 2.5 ug/mL of LNP formulation (with mRNA coding for TRC nuclease along with 125K MOI AAV carrying CAR transgene) in the presence of 0%, 0.31%, 0.625%, 1.25%, 2.5%, or 5.0% (vol/vol) of human serum.

### 3. Analysis

Flow cytometry was used to assess cell phenotype of cells at day 4. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

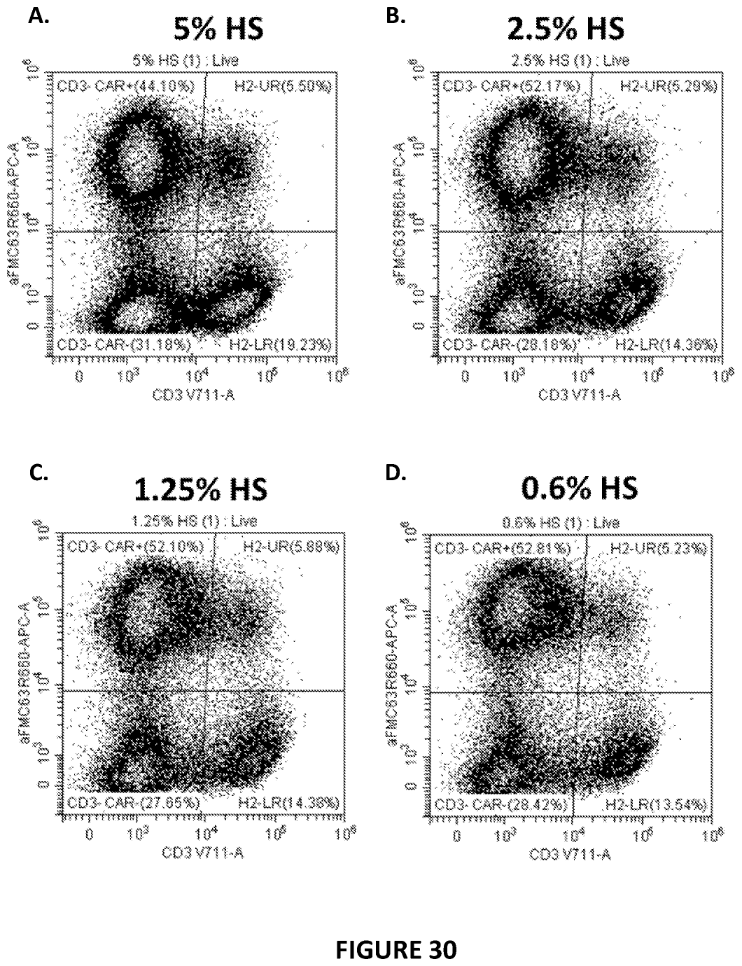

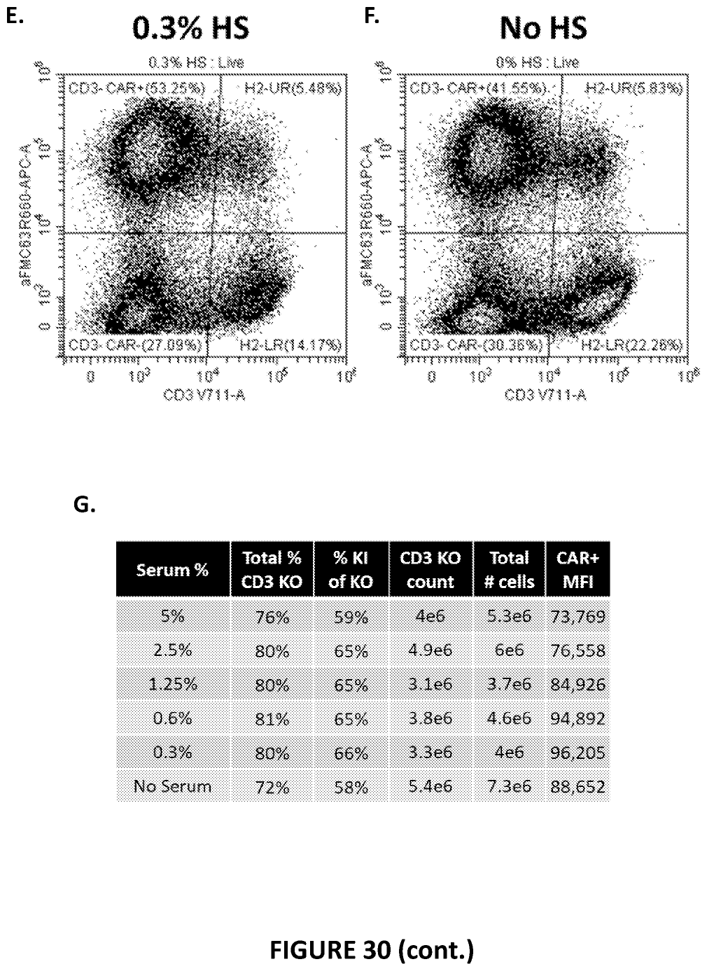

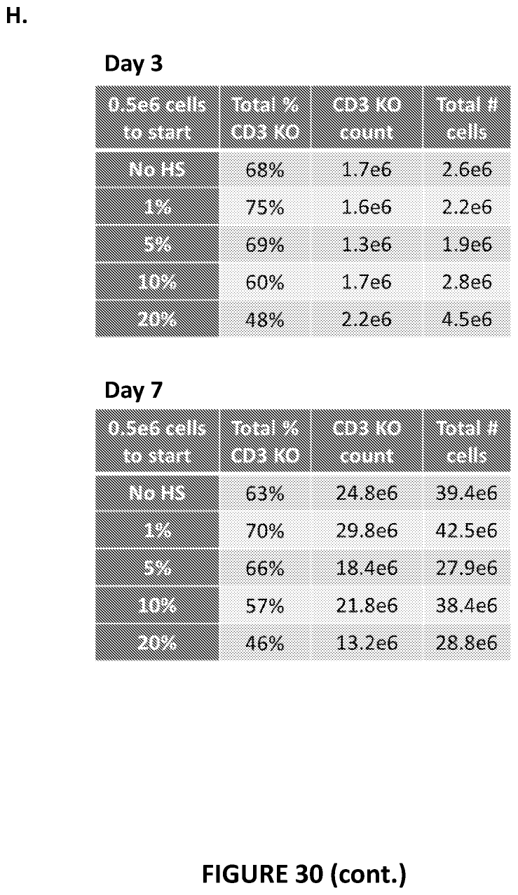

The frequency of CD3 (i.e., TCR) knockout, and the frequency of the CAR transgene knock-in, in the presence or absence of various concentrations of human serum in the culture medium is shown and summarized in the flow cytometry plots and table in FIGS. 30A-30G.

This study illustrates the ability of LNP formulations to transfect T cells with mRNA encoding an engineered nuclease, knockout the TCR locus, and allow AAV transduction and insertion of a CAR transgene, in medium supplemented with serum at concentrations at least as high as 5% (vol/vol). Therefore, this data demonstrates that certain LNP formulations, and AAV co-transfection in the presence of ApoE, does not necessarily need low serum conditions, or serum-free conditions, to maintain potency and tolerability.

A follow-up experiment was performed, essentially as described above, using the same LNP formulation in either serum-free medium, or with serum concentrations of 1%, 5%, 10%, or 20% to evaluate the effects of higher concentrations of serum on this LNP formulation (DLin-MC3-DMA, cholesterol, DSPC, and DMG-PEG (2000) at a 40:48.5:10:1.5 molar ratio). T cells were not transduced with AAV to deliver a CAR transgene for knock-in. The resulting % CD3 knockout, total number of CD3 knockout cells, and total cell numbers observed on day 3 and day 7 after introduction of the nuclease mRNA are shown in FIG. 30H. Although the highest concentration of serum did inhibit editing to some degree when compared to a serum-free or the lower percent serum conditions (1%-10%), it was observed that this particular LNP formulation was still capable of editing its target site, and knocking out the endogenous TCR (evidenced by CD3 knockout), with a high frequency (48% on day 3; 46% on day 7).

### Example 15

### Evaluation of Apolipoprotein E Isoforms

### 1. Lipid Nanoparticle Formulation

The purpose of this experiment was to evaluate multiple ApoE isoforms for use in the methods of the invention, particularly in the delivery of nuclease mRNA by LNPs in the production of CAR T cells. The lipid materials used for the formulation of LNP included DLin-MC3-DMA, cholesterol, DSPC, and DMG-PEG (2000) at a 50:38.5:10:1.5 molar ratio dissolved in ethanol at a total lipid concentration of 15 mM (Formulated at constant N:P=8). The mix of lipids was stored at −80° C. and thawed by heating to 50° C. in heat block. Lipid mix was taken off heat and vortexed immediately before use in formulation. The mRNA material coding for the TRC nuclease included a clean cap 1 structure with unmodified uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Formulation was stored frozen (−80° C.) at 1 mg/ml in 250 mM sucrose in PBS. Formulation was thawed at room temperature and diluted to desired concentration in PBS before addition to cell culture media. The formulation was added to human donor T cells to asses efficacy of LNP formulations to deliver mRNA encoding a TRC nuclease to edit at the TCR locus and reduce TCR on the cell surface, measured by CD3 staining and flow cytometry analysis.

### 2. T Cell Isolation and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum free medium. Samples of 5e5 cells/mL were treated with or without 1 ug/mL of ApoE isoforms 2, 3, or 4, or a mixture of isoforms, along with 2 ug/mL of LNP formulation with mRNA coding for TRC nuclease.

### 3. Analysis

Flow cytometry was used to assess cell phenotype of cells at day 4. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

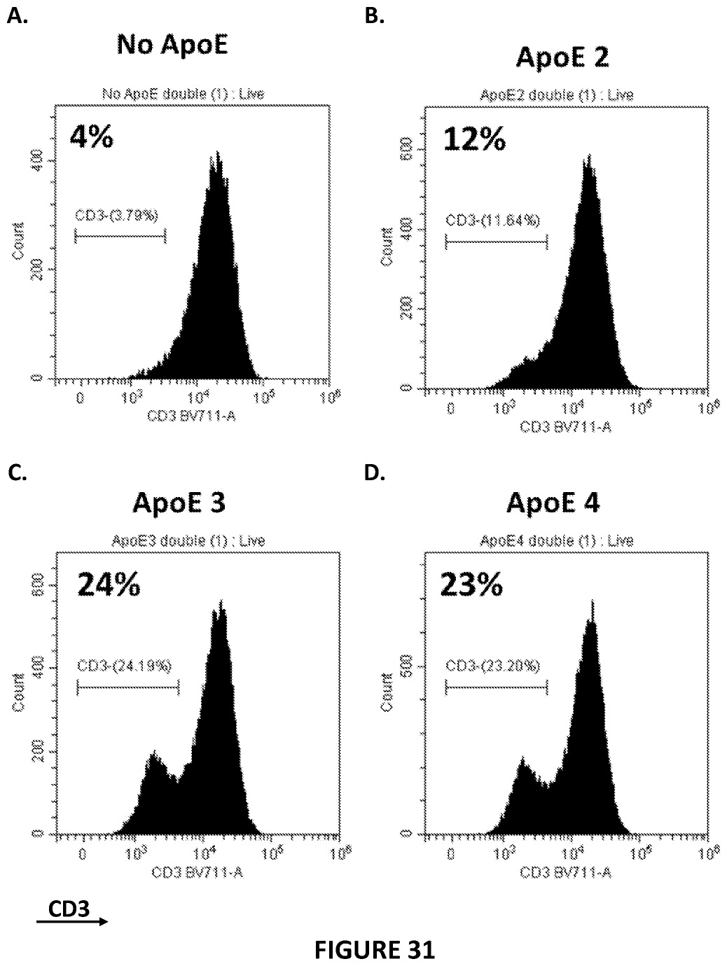

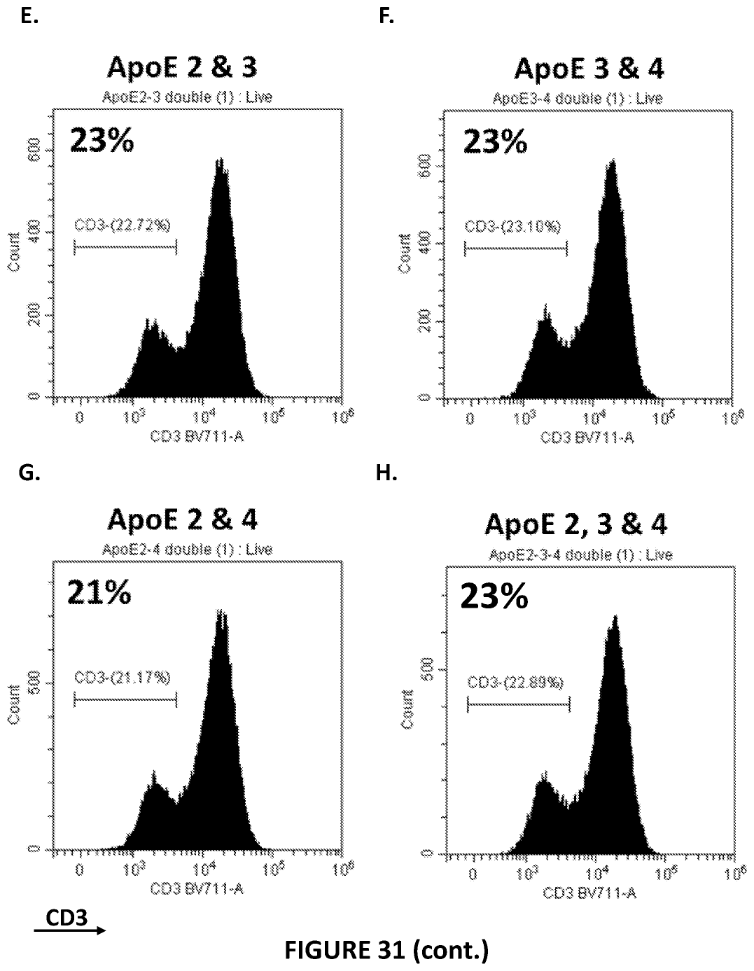

The frequency of CD3 (i.e., TCR) knockout, in the presence or absence of different isoforms of ApoE or combinations of ApoE isoforms, in the culture medium is shown in FIGS. 31A-31H. This study illustrates the ability of multiple ApoE isoforms to enhance LNP transfection of T cells with nuclease mRNA in order to knockout the TCR locus. Although ApoE isoforms 3 and 4 appeared to be more efficacious than isoform 2, equal molar mixtures of any combination of isoforms demonstrated similar efficacy compared to isoform 3 or isoform 4 alone.

### Example 16

### Enhanced LNP Transfection of Primary Human T Cells with Apolipoprotein E

### 1. Lipid Nanoparticle Formulation

The purpose of this study was to evaluate at what concentrations, and in what manner ApoE assists in the delivery of nuclease mRNA by LNPs in the production of CAR T cells. The lipid materials used for the formulation of LNP included DLin-MC3-DMA, cholesterol, DSPC, and DMG-PEG (2000) at a 50:38.5:10:1.5 molar ratio dissolved in ethanol at a total lipid concentration of 15 mM (Formulated at constant N:P=8). The mix of lipids was stored at −80° C. and thawed by heating to 50° C. in heat block. Lipid mix was taken off heat and vortexed immediately before use in formulation. The mRNA material coding for the TRC nuclease included a clean cap 1 structure with unmodified uridine. mRNA was stored at −80° C. and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. Formulation was stored frozen (−80° C.) at 1 mg/ml in 250 mM sucrose in PBS. Formulation was thawed at room temperature and diluted to desired concentration in PBS before addition to cell culture media. The formulation was added to human donor T cells to asses efficacy of LNP formulations to deliver mRNA encoding a TRC nuclease to edit at the TCR locus and reduce TCR on the cell surface, measured by CD3 staining and flow cytometry analysis.

### 2. T Cell Isolation and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum free medium. Samples of 5e5 cells/mL were treated with (0.04, 0.11, 1.0 and 3.0 ug/mL) or without ApoE3, along with dosing range of LNP at 0.3125, 0.625, 1.25, and 2.5 ug/mL with mRNA coding for TRC nuclease.

### 3. Analysis

Flow cytometry was used to assess cell phenotype of cells at day 7. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### 4. Results

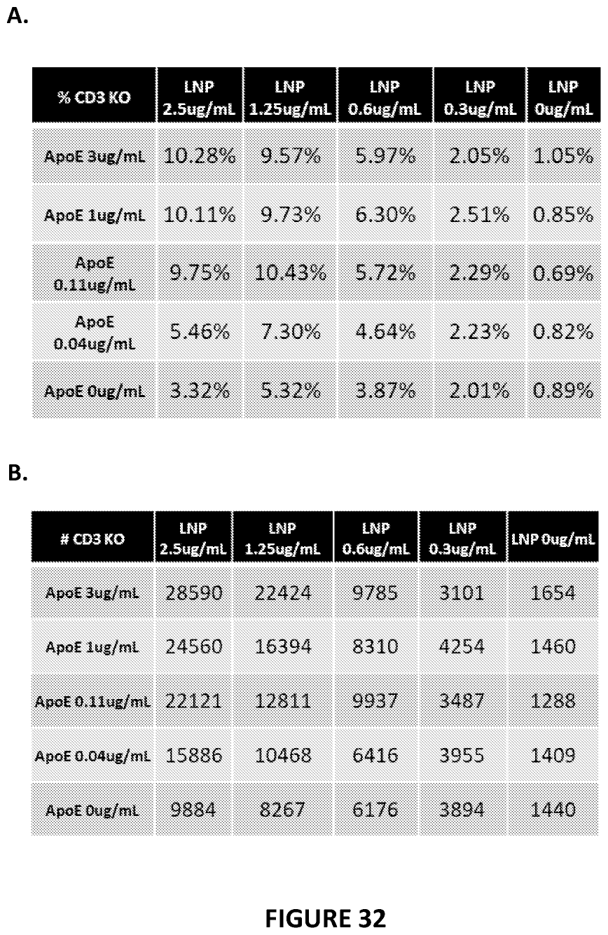

The frequency of CD3 (i.e., TCR) knockout, and the total numbers of CD3 knockout cells produced, in the presence or absence of different concentrations of ApoE in the culture medium is summarized in the tables in FIGS. 32A and 32B, respectively. This study evaluated the impact of ApoE and LNP concentrations in transfection media during LNP addition to primary human T cells. This study illustrated the ability of ApoE to enhance LNP transfection of T cells with mRNA encoding an engineered nuclease to knockout the TCR locus. It was observed that ApoE enhanced LNP transfection in a dose-dependent manner, with a saturated effect appearing at higher levels of ApoE tested.

1. A method for preparing genetically-modified immune cells, said method comprising:
contacting immune cells with lipid nanoparticles in the presence of an apolipoprotein;
wherein said lipid nanoparticles comprise a cationic lipid selected from the group consisting of DLin-DMA, DLin-MC3-DMA, DLin-KC2-DMA, DODMA, SS-OP, and derivatives thereof;
wherein said lipid nanoparticles comprise mRNA encoding an engineered nuclease having specificity for a recognition sequence in the genome of said immune cells;
wherein said mRNA is delivered into said immune cells and said engineered nuclease is expressed; and
wherein said engineered nuclease generates a cleavage site at said recognition sequence.

2. The method of claim 1, wherein said immune cells are contacted with said lipid nanoparticles in a serum-free culture condition.

3. The method of claim 1, wherein the immune cells are contacted with said lipid nanoparticles in a culture condition comprising serum at a concentration (vol/vol) of less than about 0.31%, less than about 0.625%, less than about 1.25%, less than about 2.5%, less than about 5%, or less than about 10%.

4. The method of any one of claims 1-3, wherein said method is performed in vitro.

5. The method of any one of claims 1-4, wherein said immune cells are human immune cells.

6. The method of any one of claims 1-5, wherein said immune cells are T cells, or cells derived therefrom, natural killer (NK) cells, or cells derived therefrom, or B cells, or cells derived therefrom.

7. The method of any one of claims 1-6, wherein said apolipoprotein is present at a concentration between 0.01 μg/mL to 10 μg/mL.

8. The method of any one of claims 1-7, wherein said apolipoprotein is present at a concentration of about 1 μg/mL.

9. The method of any one of claims 1-8, wherein said apolipoprotein is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)) protein.

10. The method of any one of claims 1-9, wherein said apolipoprotein is ApoE.

11. The method of any one of claims 1-10, wherein said lipid nanoparticles do not comprise an immune cell targeting molecule.

12. The method of any one of claims 1-11, wherein said recognition sequence is in a target gene, and wherein expression of a polypeptide encoded by said target gene is disrupted by non-homologous end joining at said cleavage site.

13. The method of claim 12, wherein said target gene is a T cell receptor (TCR) alpha gene or a TCR alpha constant region gene.

14. The method of claim 12 or claim 13, wherein said genetically-modified immune cells do not have detectable cell-surface expression of an endogenous alpha/beta TCR.

15. The method of any one of claims 12-14, wherein said method produces a population wherein between about 5% and about 70% of said genetically-modified immune cells in said population do not have detectable cell-surface expression of an endogenous alpha/beta TCR.

16. The method of any one of claims 1-15, wherein said genetically-modified immune cells express a chimeric antigen receptor (CAR) or exogenous TCR.

17. The method of any one of claims 1-16, wherein said immune cells are contacted with:
(a) a first population of lipid nanoparticles comprising mRNA encoding a first engineered nuclease having specificity for a first recognition sequence; and
(b) a second population of lipid nanoparticles comprising mRNA encoding a second engineered nuclease having specificity for a second recognition sequence;
wherein said first engineered nuclease and said second engineered nuclease are expressed in said immune cells, and wherein said first engineered nuclease generates a first cleavage site in said first recognition sequence and said second engineered nuclease generates a second cleavage site in said second recognition sequence.

18. The method of claim 17, wherein said first recognition sequence and said second recognition sequence are in the same target gene, and wherein expression of a polypeptide encoded by said target gene is disrupted by non-homologous end joining at said first cleavage site and said second cleavage site.

19. The method of claim 17, wherein said first recognition sequence and said second recognition sequence are in different target genes, wherein expression of polypeptides encoded by said different target genes is disrupted by non-homologous end joining at said first cleavage site and said second cleavage site.

20. The method of claim 19, wherein said different target genes are a human TCR alpha constant region gene and a human beta-2 microglobulin gene, and wherein said genetically-modified immune cells do not have detectable cell-surface expression of an endogenous TCR or beta-2 microglobulin.

21. The method of any one of claims 1-11, wherein said method further comprises introducing into said immune cells a template nucleic acid comprising an exogenous polynucleotide, wherein said exogenous polynucleotide is inserted into the genome of said immune cells at said cleavage site.

22. The method of claim 21, wherein said recognition sequence is in a target gene, and wherein insertion of said exogenous polynucleotide disrupts expression of a polypeptide encoded by said target gene.

23. The method of claim 22, wherein said target gene is a TCR alpha gene or a TCR alpha constant region gene.

24. The method of claim 22 or claim 23, wherein said target gene is a TCR alpha constant region gene, and wherein said genetically-modified immune cells do not have detectable cell-surface expression of an endogenous TCR.

25. The method of any one of claims 21-24, wherein said exogenous polynucleotide encodes a polypeptide of interest.

26. The method of any one of claims 21-25, wherein said exogenous polynucleotide encodes a CAR or an exogenous TCR.

27. The method of any one of claims 21-26, wherein said template nucleic acid is introduced into said immune cells using a recombinant DNA construct.

28. The method of claim 27, wherein said recombinant DNA construct is encapsulated in a lipid nanoparticle.

29. The method of any one of claims 21-26, wherein said template nucleic acid is introduced into said immune cells using a recombinant virus.

30. The method of claim 29, wherein said recombinant virus is a recombinant adenovirus, a recombinant lentivirus, a recombinant retrovirus, or a recombinant adeno-associated virus (AAV).

31. The method of claim 29 or claim 30, wherein said recombinant virus is a recombinant AAV.

32. The method of any one of claims 21-31, wherein said template nucleic acid is introduced into said immune cells within 48 hours after said immune cells are contacted with said lipid nanoparticles

33. The method of any one of claims 21-31, wherein said template nucleic acid is introduced into said immune cells within 12 hours prior to when said immune cells are contacted with said lipid nanoparticles.

34. The method of any one of claims 21-31, wherein said template nucleic acid is introduced into said immune cells between 0-24 hours or between 24-48 hours, after said immune cells are contacted with said lipid nanoparticles.

35. The method of any one of claims 21-34, wherein said immune cells are not transferred to a new vessel between said step of contacting and said step of introducing.

36. The method of any one of claims 21-35, wherein said immune cells are not centrifuged between said step of contacting and said step of introducing.

37. The method of any one of claims 1-36, wherein said genetically-modified immune cells are genetically-modified T cells, or cells derived therefrom, expressing a CAR or exogenous TCR.

38. The method of claim 37, wherein said genetically-modified T cells do not have detectable cell-surface expression of an endogenous alpha/beta TCR.

39. The method of claim 37 or claim 38, wherein said method produces a population of genetically-modified T cells having a CD4+ T cell to CD8+ T cell ratio of between about 0.8 and about 1.6 when cultured for one to two weeks after said contacting step.

40. The method of any one of claims 37-39, wherein said method produces a population of genetically-modified T cells wherein between about 65% and about 84% of CD4+ T cells in said population exhibit a central memory phenotype when cultured for one to two weeks after said contacting step.

41. The method of any one of claims 37-40, wherein said method produces a population of genetically-modified T cells wherein about 3% to about 10% of CD4+ T cells in said population exhibit an effector phenotype when cultured for one to two weeks after said contacting step.

42. The method of any one of claims 1-41, wherein the molar concentration of said cationic lipid is from about 20% to about 80%, from about 30% to about 70%, from about 40% to about 60%, from about 45% to about 55%, or about 50% of the total lipid molar concentration.

43. The method of any one of claims 1-42, wherein the molar concentration of said cationic lipid is about 40%, about 50%, or about 60% of the total lipid molar concentration.

44. The method of any one of claims 1-43, wherein said lipid nanoparticles comprise a molar ratio of cationic lipid to mRNA of from about 1 to about 20, from about 2 to about 16, from about 4 to about 12, from about 6 to about 10, or about 8.

45. The method of any one of claims 1-44, wherein said lipid nanoparticles comprise a molar ratio of cationic lipid to mRNA of about 8.

46. The method of any one of claims 1-45, wherein said lipid nanoparticles comprise:
(a) one or more non-cationic lipids; and
(b) a lipid conjugate.

47. The method of claim 46, wherein the molar concentration of said non-cationic lipids is from about 20% to about 80%, from about 30% to about 70%, from about 40% to about 70%, from about 40% to about 60%, from about 46% to about 50% of the total lipid molar concentration.

48. The method of claim 46 or claim 47, wherein the molar concentration of said non-cationic lipids is about 40%, about 48.5%, about 50%, or about 60% of the total lipid molar concentration.

49. The method of any one of claims 46-48, wherein said non-cationic lipids comprise a phospholipid, wherein the molar concentration of said phospholipid is from about 0% to about 30%, from about 2.5% to about 25%, from about 5% to about 20%, from about 5% to about 15%, from about 7.5% to about 12.5%, or about 10% of the total lipid molar concentration.

50. The method of claim 49, wherein the molar concentration of said phospholipid is about 10% or about 20% of the total lipid molar concentration.

51. The method of claim 49 or claim 50, wherein said phospholipid is DSPC.

52. The method of any one of claims 46-51, wherein said non-cationic lipids comprise a steroid, wherein the molar concentration of said steroid is from about 20% to about 60%, from about 25% to about 55%, from about 30% to about 50%, from about 35% to about 40%, or about 38.5% of the total lipid molar concentration.

53. The method of claim 52, wherein the molar concentration of said steroid is about 30%, about 38.5%, or about 50% of the total lipid molar concentration.

54. The method of claim 52 or claim 53, wherein said steroid is cholesterol.

55. The method of any one of claims 46-54, wherein the molar concentration of said lipid conjugate is from about 0.01% to about 10%, from about 0.2% to about 8%, from about 0.5% to about 5%, from about 0.1% to about 1.5%, from about 1% to about 2%, or about 1.5% of the total lipid molar concentration.

56. The method of any one of claims 46-55, wherein the molar concentration of said lipid conjugate is about 1.5% of the total lipid molar concentration.

57. The method of any one of claims 46-56, wherein said lipid conjugate is a pegylated lipid.

58. The method of any one of claims 46-57, wherein said lipid conjugate is a DMG-PEG.

59. The method of any one of claims 46-58, wherein said lipid conjugate is DMG-PEG2000 or DMG-PEG5000.

60. The method of any one of claims 49-59, wherein a molar ratio of said cationic lipid to said phospholipid is from about 1:1 to about 20:1, about 6:1 to about 20:1, about 10:1 to about 20:1, about 16:1 to about 20:1, or about 2:1 to about 7:1.

61. The method of claim 60, wherein a molar ratio of said cationic lipid to said phospholipid is from about 2:1 to about 7:1.

62. The method of claim 60 or claim 61, wherein a molar ratio of said cationic lipid to said phospholipid is about 2:1, about 4:1, about 5:1, or about 6:1.

63. The method of any one of claims 52-62, wherein a molar ratio of said cationic lipid to said steroid is from about 0.25:1 to about 5:1, about 0.5:1 to about 5:1, about 0.75:1 to about 5:1, about 2:1 to about 5:1, or about 0.8:1 to about 2:1.

64. The method of claim 63, wherein a molar ratio of said cationic lipid to said steroid is from about 0.8:1 to about 2:1.

65. The method of claim 64 or claim 65, wherein a molar ratio of said cationic lipid to said steroid is about 0.8:1, about 1.3:1, about 1:1, or about 2:1.

66. The method of any one of claims 46-65, wherein a molar ratio of said cationic lipid to said lipid conjugate is from about 10:1 to about 1000:1, about 25:1 to about 1000:1, about 75:1 to about 1000:1, about 400:1 to about 1000:1, about 550:1 to about 1000:1, about 20:1 to about 600:1, or about 25:1 to about 400:1.

67. The method of claim 66, wherein a molar ratio of said cationic lipid to said lipid conjugate is from about 25:1 to about 400:1.

68. The method of claim 66 or claim 67, wherein a molar ratio of said cationic lipid to said lipid conjugate is about 25:1, about 33:1, about 60:1, or about 400:1.

69. The method of any one of claims 52-68, wherein a molar ratio of said steroid to said lipid conjugate is from about 25:1 to about 750:1, about 50:1 to about 750:1, about 100:1 to about 750:1, about 150:1 to about 750:1, about 200:1 to about 750:1, about 250:1 to about 750:1, about 300:1 to about 750:1, about 350:1 to about 750:1, about 400:1 to about 750:1, about 450:1 to about 750:1, about 500:1 to about 750:1, about 10:1 to about 500:1, or about 25:1 to about 500:1.

70. The method of claim 69, wherein a molar ratio of said steroid to said lipid conjugate is from about 25:1 to about 500:1.

71. The method of claim 69 or claim 70, wherein a molar ratio of said steroid to said lipid conjugate is from about 25:1, about 30:1, or about 500:1.

72. The method of any one of claims 49-71, wherein a molar ratio of said phospholipid to said lipid conjugate is from about 1:1 to about 300:1, about 50:1 to about 300:1, about 100:1 to about 300:1, about 125:1 to about 300:1, about 150:1 to about 300:1, about 175:1 to about 300:1, about 200:1 to about 300:1, about 225:1 to about 300:1, about 250:1 to about 300:1, about 275:1 to about 300:1, about 3:1 to about 200:1, or about 5:1 to about 100:1.

73. The method of claim 72, wherein a molar ratio of said phospholipid to said lipid conjugate is from about 5:1 to about 100:1.

74. The method of claim 72 or claim 73, wherein a molar ratio of said phospholipid to said lipid conjugate is about 6:1, about 10:1, about 13:1 or about 100:1.

75. The method of any one of claims 1-74, wherein said lipid nanoparticles comprise:
(a) said cationic lipid at a molar concentration of about 30% to about 60% the total lipid molar concentration;
(b) a steroid at a molar concentration of about 20% to about 60% of the total lipid molar concentration;
(c) a phospholipid at a molar concentration of about 5% to about 20% of the total lipid molar concentration; and
(d) a lipid conjugate at a molar concentration of about 0.10% to about 1.5% of the total lipid molar concentration.

76. The method of any one of claims 1-74, wherein said lipid nanoparticles comprise:
(a) said cationic lipid at a molar concentration of about 40% of the total lipid molar concentration;
(b) a steroid at a molar concentration of about 38.5% of the total lipid molar concentration;
(c) a phospholipid at a molar concentration of about 20% of the total lipid molar concentration; and
(d) a lipid conjugate at a molar concentration of about 1.5% of the total lipid molar concentration.

77. The method of any one of claims 1-74, wherein said lipid nanoparticles comprise:
(a) said cationic lipid at a molar concentration of about 50% of the total lipid molar concentration;
(b) a steroid at a molar concentration of about 38.5% of the total lipid molar concentration;
(c) a phospholipid at a molar concentration of about 10% of the total lipid molar concentration; and
(d) a lipid conjugate at a molar concentration of about 1.5% of the total lipid molar concentration.

78. The method of any one of claims 1-74, wherein said lipid nanoparticles comprise:
(a) said cationic lipid at a molar concentration of about 60% of the total lipid molar concentration;
(b) a steroid at a molar concentration of about 29% of the total lipid molar concentration;
(c) a phospholipid at a molar concentration of about 10% of the total lipid molar concentration; and
(d) a lipid conjugate at a molar concentration of about 1% of the total lipid molar concentration.

79. The method of any one of claims 1-74, wherein said lipid nanoparticles comprise:
(a) said cationic lipid at a molar concentration of about 40% of the total lipid molar concentration;
(b) a steroid at a molar concentration of about 48.5% of the total lipid molar concentration;
(c) a phospholipid at a molar concentration of about 10% of the total lipid molar concentration; and
(d) a lipid conjugate at a molar concentration of about 1.5% of the total lipid molar concentration.

80. The method of any one of claims 1-74, wherein said lipid nanoparticles comprise:
(a) said cationic lipid at a molar concentration of about 40% of the total lipid molar concentration;
(b) a steroid at a molar concentration of about 49.9% of the total lipid molar concentration;
(c) a phospholipid at a molar concentration of about 10% of the total lipid molar concentration; and
(d) a lipid conjugate at a molar concentration of about 0.10% of the total lipid molar concentration.

81. The method of any one of claims 1-74, wherein said lipid nanoparticles comprise DLin-MC3-DMA, DSPC, cholesterol, and DMG-PEG2000 at a molar ratio of about 50:10:38.5:1.5 or about 40:10:48.5:1.50.

82. The method of any one of claims 1-74, wherein said lipid nanoparticles comprise DLin-MC3-DMA, DSPC, cholesterol, and DMG-PEG5000 at a molar ratio of about 40:10:49.90:0.10.

83. The method of any one of claims 1-74, wherein said lipid nanoparticles comprise DLin-MC3-DMA, DOPC, cholesterol, and DMG-PEG2000 at a molar ratio of about 40:20:38.5:1.5 or about 60:10:29:1.

84. The method of any one of claims 1-74, wherein said lipid nanoparticles comprise DODMA, DSPC, cholesterol, and DMG-PEG2000 at a molar ratio of about 50:10:38.5:1.5.

85. The method of any one of claims 75-80, wherein said cationic lipid is DLin-MC3-DMA, said steroid is cholesterol, said phospholipid is DSPC, and said lipid conjugate is PEG 5000.

86. The method of any one of claims 75-80, wherein said cationic lipid is DLin-MC3-DMA, said steroid is cholesterol, said phospholipid is DSPC, and said lipid conjugate is PEG 2000.

87. The method of any one of claims 75-80, wherein said cationic lipid is DLin-MC3-DMA, said steroid is cholesterol, said phospholipid is DOPC, and said lipid conjugate is PEG 2000.

88. The method of any one of claims 1-87, wherein said lipid nanoparticles have a size from about 50 nm to about 300 nm or from about 60 nm to about 120 nm.

89. The method of any one of claims 1-88, wherein the polydispersity index of said lipid nanoparticles is less than about 0.3 or less than about 0.2.

90. The method of any one of claims 1-89, wherein the zeta potential of said lipid nanoparticles is from about −40 mV to about 40 mV or from about −10 mV to about 10 mV.

91. The method of any one of claims 1-90, wherein said engineered nuclease is an engineered meganuclease, a zinc finger nuclease, a TALEN, a compact TALEN, a CRISPR system nuclease, or a megaTAL.

92. The method of any one of claims 1-91, wherein said engineered nuclease is an engineered meganuclease.

93. The method of any one of claims 1-92, wherein said lipid nanoparticle does not comprise a T cell targeting molecule.

94. The method of any one of claims 1-93, wherein said mRNA comprises a 5′ cap selected from the group consisting of an Anti-Reverse Cap Analog (ARCA) cap, a 7-methyl-guanosine (7mG) cap, a CleanCap® analog, a vaccinia cap, and analogs thereof.

95. The method of any one of claims 1-94, wherein said mRNA comprises at least one nucleoside modification.

96. The method of claim 95, wherein said nucleoside modification is selected from the group consisting of a modification from uridine to pseudouridine and uridine to N1-methyl pseudouridine.

97. The method of claim 95 or claim 96, wherein said nucleoside modification is from uridine to pseudouridine.

98. The method of any one of claims 1-94, wherein said mRNA does not comprise a nucleoside modification.

99. A population of genetically-modified immune cells prepared according to the method of any one of claims 1-98.

100. A population of genetically-modified immune cells that are electroporation naïve, wherein said genetically-modified immune cells comprise a target gene modified by an engineered nuclease to disrupt expression of an endogenous polypeptide encoded by said target gene.

101. The population of claim 100, wherein said genetically-modified immune cells are genetically-modified T cells, genetically-modified NK cells, or genetically-modified B cells.

102. The population of claim 100 or claim 101, wherein said genetically-modified immune cells are genetically-modified human T cells.

103. The population of any one of claims 100-102, wherein said genetically-modified immune cells comprise a nucleic acid sequence encoding a CAR or an exogenous TCR, wherein said CAR or exogenous TCR is expressed by said genetically-modified immune cell.

104. A population of immune cells, wherein between about 5% and about 80% of said immune cells in said population are said genetically-modified immune cells prepared by the method of any one of claims 1-98, wherein said genetically-modified immune cells comprise a disrupted TCR alpha gene or a disrupted TCR alpha constant region gene.

105. A population of immune cells, wherein between about 5% and about 65% of the immune cells in said population are said genetically-modified immune cells prepared by the method of any one of claims 1-98, wherein said genetically-modified immune cells comprise a disrupted TCR alpha gene or a disrupted TCR alpha constant region gene and express a chimeric antigen receptor or an exogenous TCR.

106. The population of claim 104 or claim 105, wherein said genetically-modified immune cells are genetically-modified T cells, genetically-modified NK cells, or genetically-modified B cells.

107. The population of any one of claims 104-106, wherein said genetically-modified immune cells are genetically-modified human T cells.

108. A pharmaceutical composition comprising a pharmaceutically-acceptable carrier and said population of genetically-modified immune cells of any one of claims 100-104.

109. A pharmaceutical composition comprising a pharmaceutically-acceptable carrier and said population of immune cells of any one of claims 104-107.

110. A method of treating a disease in a subject in need thereof, said method comprising administering to said subject a therapeutically-effective amount of said population of genetically-modified immune cells of any one of claims 99-103.

111. The method of claim 110, wherein said method comprises administering to said subject said pharmaceutical composition of claim 108.

112. A method of treating a disease in a subject in need thereof, said method comprising administering to said subject a therapeutically-effective amount of said population of immune cells of any one of claims 104-107.

113. The method of claim 112, wherein said method comprises administering to said subject said pharmaceutical composition of claim 109.

114. The method of any one of claims 110-113, wherein said method is an immunotherapy for the treatment of a cancer in a subject in need thereof, wherein said genetically-modified immune cells are genetically-modified human T cells, or cells derived therefrom, or genetically-modified NK cells, or cells derived therefrom, and wherein said genetically-modified immune cells express a CAR or an exogenous TCR, and wherein said genetically-modified immune cells do not have detectable cell-surface expression of an endogenous alpha/beta TCR.

115. The method of claim 114, wherein said cancer is selected from the group consisting of a cancer of carcinoma, lymphoma, sarcoma, blastomas, and leukemia.

116. The method of claim 114 or claim 115, wherein said cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyo sarcoma, leukemia, and Hodgkin lymphoma.

117. The method of claim 116, wherein said cancer of B-cell origin is selected from the group consisting of B-lineage acute lymphoblastic leukemia, B-cell chronic lymphocytic leukemia, B-cell non-Hodgkin lymphoma, and multiple myeloma.

118. A lipid nanoparticle composition comprising:
(a) a cationic lipid at a molar concentration of about 40%, about 50%, or about 60% of the total lipid molar concentration, wherein the cationic lipid is selected from the group consisting of DLin-MC3-DMA, DLin-KC2-DMA, DODMA, SS-OP, and derivatives thereof;
(b) a steroid at a molar concentration of about 29%, about 38.5%, about 48.5%, or about 49.9% of the total lipid molar concentration;
(c) a phospholipid at a molar concentration about 10% or about 20% of the total lipid molar concentration; and
(d) a lipid conjugate at a molar concentration of about 0.10% or about 1.5% of the total lipid molar concentration.

119. The composition of claim 118, wherein said lipid nanoparticles comprise:
(a) said cationic lipid at a molar concentration of about 40% of the total lipid molar concentration;
(b) said steroid at a molar concentration of about 38.5% of the total lipid molar concentration;
(c) said phospholipid at a molar concentration of about 20% of the total lipid molar concentration; and
(d) said lipid conjugate at a molar concentration of about 1.5% of the total lipid molar concentration.

120. The composition of claim 118, wherein said lipid nanoparticles comprise:
(a) said cationic lipid at a molar concentration of about 50% of the total lipid molar concentration;
(b) said steroid at a molar concentration of about 38.5% of the total lipid molar concentration;
(c) said phospholipid at a molar concentration of about 10% of the total lipid molar concentration; and
(d) said lipid conjugate at a molar concentration of about 1.5% of the total lipid molar concentration.

121. The composition of claim 118, wherein said lipid nanoparticles comprise:
(a) said cationic lipid at a molar concentration of about 60% of the total lipid molar concentration;
(b) said steroid at a molar concentration of about 29% of the total lipid molar concentration;
(c) said phospholipid at a molar concentration of about 10% of the total lipid molar concentration; and
(d) said lipid conjugate at a molar concentration of about 1% of the total lipid.

122. The composition of claim 118, wherein said lipid nanoparticles comprise:
(a) said cationic lipid at a molar concentration of about 40% of the total lipid molar concentration;
(b) said steroid at a molar concentration of about 48.5% of the total lipid molar concentration;
(c) said phospholipid at a molar concentration about 10% of the total lipid molar concentration; and
(d) said lipid conjugate at a molar concentration of about 1.5% of the total lipid molar concentration.

123. The composition of claim 118, wherein said lipid nanoparticles comprise:
(a) said cationic lipid at a molar concentration of about 40% of the total lipid molar concentration;
(b) said steroid at a molar concentration of about 49.9% of the total lipid molar concentration;
(c) said phospholipid at a molar concentration of about 10% of the total lipid molar concentration; and
(d) said lipid conjugate at a molar concentration of about 0.10% of the total lipid molar concentration.

124. The composition of any one of claims 118-123, wherein a molar ratio of said cationic lipid to said steroid is about 0.8:1, about 1.3:1, about 1:1, or about 2:1.

125. The composition of any one of claims 118-124, wherein a molar ratio of said cationic lipid to said phospholipid is from about 2:1, about 4:1, about 5:1, or about 6:1.

126. The composition of any one of claims 118-125, wherein a molar ratio of said cationic lipid to said lipid conjugate is about 25:1, about 33:1, about 60:1, or about 400:1.

127. The composition of any one of claims 118-126, wherein a molar ratio of said steroid to said lipid conjugate is from about 25:1, about 30:1, or about 500:1.

128. The composition of any one of claims 118-127, wherein a molar ratio of said phospholipid to said lipid conjugate is about 6:1, about 10:1, about 13:1 or about 100:1.

129. The composition of any one of claims 118-128, wherein said cationic lipid is DLin-MC3-DMA, said steroid is cholesterol, said phospholipid is DSPC, and said lipid conjugate is PEG 5000.

130. The composition of any one of claims 118-129, wherein said cationic lipid is DLin-MC3-DMA, said steroid is cholesterol, said phospholipid is DSPC, and said lipid conjugate is PEG 2000.

131. The composition of any one of claims 118-130, wherein said cationic lipid is DLin-MC3-DMA, said steroid is cholesterol, said phospholipid is DOPC, and said lipid conjugate is PEG 2000.

132. The composition of claim 118, wherein said lipid nanoparticles comprise DLin-MC3-DMA, DSPC, cholesterol, and DMG-PEG2000 at a molar ratio of about 50:10:38.5:1.5 or about 40:10:48.5:1.50.

133. The composition of claim 118, wherein said lipid nanoparticles comprise DLin-MC3-DMA, DSPC, cholesterol, and DMG-PEG5000 at a molar ratio of about 40:10:49.90:0.10.

134. The composition of claim 118, wherein said lipid nanoparticles comprise DLin-MC3-DMA, DOPC, cholesterol, and DMG-PEG2000 at a molar ratio of about 40:20:38.5:1.5 or about 60:10:29:1.

135. The composition of claim 118, wherein said lipid nanoparticles comprise DODMA, DSPC, cholesterol, and DMG-PEG2000 at a molar ratio of about 50:10:38.5:1.5.

136. The composition of any one of claims 118-135, wherein said lipid nanoparticles further comprises an mRNA encoding an engineered nuclease having specificity for a recognition sequence in the genome of an immune cell.

137. The composition of claim 136, wherein said mRNA comprises a 5′ cap selected from the group consisting of an Anti-Reverse Cap Analog (ARCA) cap, a 7-methyl-guanosine (7mG) cap, a CleanCap® analog, a vaccinia cap, and analogs thereof.

138. The composition of claim 136 or claim 137, wherein said mRNA comprises at least one nucleoside modification.

139. The composition of claim 138, wherein said nucleoside modification is selected from the group consisting of a modification from uridine to pseudouridine and uridine to N1-methyl pseudouridine.

140. The composition of claim 138 or claim 139, wherein said nucleoside modification is from uridine to pseudouridine.

141. The composition of claim 136 or claim 137, wherein said mRNA does not comprise a nucleoside substitution.

142. The composition of any one of claims 118-141, wherein said lipid nanoparticles have a size from about 50 nm to about 300 nm, or from about 60 nm to about 120 nm.

143. The composition of any one of claims 118-142, wherein the polydispersity index of said lipid nanoparticles is less than about 0.3, or less than about 0.2.

144. The composition of any one of claims 118-143, wherein the zeta potential of said lipid nanoparticles is from about −40 mV to about 40 mV or from about −10 mV to about 10 mV.

145. The composition of any one of claims 118-144, wherein said lipid nanoparticles comprise a molar ratio of cationic lipid to mRNA of from about 1 to about 20, from about 2 to about 16, from about 4 to about 12, from about 6 to about 10, or about 8.

146. The composition of any one of claims 118-145, wherein said lipid nanoparticles comprise a molar ratio of cationic lipid to mRNA of about 8.

147. The composition of any one of claims 118-146, wherein said lipid nanoparticles do not comprise an immune cell targeting molecule.

148. The composition of any one of claims 118-147, wherein said lipid nanoparticles do not comprise a T cell targeting molecule.

149. A kit for transfecting a eukaryotic cell with mRNA comprising:
(a) an apolipoprotein; and
(b) a lipid nanoparticle composition according to any one of claims 110-138.

150. The kit of claim 149, wherein said apolipoprotein is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)) protein.

151. The kit of claim 149 or claim 150, wherein said apolipoprotein is ApoE.

152. The kit of any one of claims 149-151, wherein said apolipoprotein and said lipid nanoparticle composition are provided together in one vial or are provided separately in two or more vials.


SEQUENCE LISTING (4 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 3 - DNA, 22 bp, Homo sapiens
tggcctggagcaacaaatctga

SEQ ID NO: 4 - DNA, 22 bp, Homo sapiens
accggacctcgttgtttagact